(19) United States
(12) Patent Application Publication
(10) Pub. No.: US 2026/0356785 A1
(43) Pub. Date: Nov. 12, 2026

(54) LIGAND-ENABLED TRANSANNULAR C-H FUNCTIONALIZATION OF CYCLOALKANE CARBOXYLIC ACIDS

(71) Applicant: THE SCRIPPS RESEARCH INSTITUTE, La Jolla, CA (US)

(72) Inventors: Jin-Quan YU, San Diego, CA (US); Guowei KANG, San Diego, CA (US)

(73) Assignee: THE SCRIPPS RESEARCH INSTITUTE, La Jolla, CA (US)

(21) Appl. No.: 19/153,865

(22) PCT Filed: Feb. 5, 2024
(86) PCT No.: PCT/US2024/014386

§ 371 Date: Aug. 5, 2025

Related U.S. Application Data
(60) Provisional application No. 63/483,314, filed on Feb. 6, 2023.

(51) Int. Cl. C07C 51/347; C07C 51/363; C07C 51/367; C07C 51/373; C07C 67/313; C07C 201/12; C07C 253/30; C07D 207/327; C07D 209/18; C07D 213/55; C07D 213/61; C07D 215/14; C07D 221/14; C07D 231/12; C07D 231/56; C07D 239/52; C07D 239/74; C07D 277/64; C07D 309/08; C07D 333/24; C07D 453/02

(52) U.S. Cl. CPC C07C 51/347; C07C 51/363; C07C 51/367; C07C 51/373; C07C 67/313; C07C 201/12; C07C 253/30; C07D 207/327; C07D 209/18; C07D 213/55; C07D 213/61; C07D 215/14; C07D 221/14; C07D 231/12; C07D 231/56; C07D 239/52; C07D 239/74; C07D 277/64; C07D 309/08; C07D 333/24; C07D 453/02; C07C 2601/08; C07C 2601/14; C07C 2601/18; C07C 2602/08; C07C 2602/24

(57) ABSTRACT

This application discloses methods of transannular γ-methylene C(sp³)-H arylation of cycloalkane carboxylic acids using novel quinuclidine-pyridone ligand catalysts.

### CROSS REFERENCE TO RELATED APPLICATION

This application claims priority to U.S. provisional patent application No. 63/483,314, which was filed on Feb. 6, 2023, and which is hereby incorporated by reference in its entirety.

### GOVERNMENT SUPPORT

This invention was made with government support under GM084019 awarded by the National Institutes of Health. The government has certain rights in the invention.

### FIELD OF THE INVENTION

This application relates to methods of transannular γ-methylene C(sp³)-H arylation of cycloalkane carboxylic acids using novel quinuclidine-pyridone ligand catalysts.

### BACKGROUND OF THE INVENTION

Cyclic structures are vitally important in medicinal chemistry since they provide control over molecular shape while reducing the overall number of rotatable bonds, offering increased oral bioavailability, and providing enhanced control over the activity, specificity, and physical properties of drug candidates¹⁻³. Likewise, rings are ubiquitous among natural products⁴, highlighting the importance of cyclic systems in biology. Organic chemists have often relied on powerful cycloadditions, typified by the Diels-Alder reaction, or intramolecular cyclizations to assemble ring systems from acyclic precursors⁵. However, the synthesis of those precursors frequently requires multiple steps, and the cyclization reactions themselves can be challenging to promote and control⁶. As an alternative, complex carbocycles can be constructed through the modification of pre-formed cyclic starting materials, such as cycloalkanones or terpenoid chiral pool compounds⁷. However, elaboration of these starting materials by classical methods can necessitate lengthy synthetic sequences to relay reactivity from pre-existing functionality to the desired site of functionalization. In principle C—H activation offers an alternative disconnection that would bypass this limitation and enable rapid and efficient access to a diverse range of functionalized (poly)cyclic scaffolds, but the general applicability of this approach is predicated on the development of methods for the site-selective functionalizations of both proximal and remote C—H bonds (FIG. 1A). The former are typically far more reactive⁸, so achieving selectivity for the functionalization of remote positions in the presence of competing proximal C—H bonds remains a major challenge. This is particularly true for cyclic systems, where the activation of remote sites requires transannular C—H cleavage to form strained, bridged bicyclic palladacycles. Recent advances in macrocyclophane-based directing templates have provided a solution for controlling remote C(sp²)-H activation of (hetero)aromatic rings⁹⁻¹⁴. However, methods for the C—H functionalization of saturated carbocycles—a focus of modern drug discovery efforts¹⁵—have largely been limited to the activation of proximal sites¹⁶⁻¹⁸. Some transannular C—H activations have been reported^{6,19-33}, but the vast majority of these proceed via 5-membered palladacycles^{19, 21, 22, 24, 25, 27-32}, necessitating the presence of a nitrogen atom within or directly attached to the ring to be able to reach remote C—H bonds (FIG. 1B). Functionalizing remote C—H bonds via 6-membered palladacycles would provide a much more general solution, however, only a few such examples have been reported^{6,20,23,26,33}, often with modest site-selectivity or specialized bicyclic substrates. Moreover, because of the challenging nature of methylene C—H activation, nearly all previous reports of transannular functionalizations employ pre-installed bidentate directing groups^{6,19-21, 23, 25-28, 30-32}, limiting the scope of these transformations and the opportunities for downstream derivatization of the products.

Hence, there exists a need in the field to develop a general and native-functional-group-directed method for the transannular functionalizations of simple, monocyclic substrates.

Herein described is the molecular editing of saturated carbocyclic scaffolds: the development of γ-selective transannular C—H arylation of a wide range of small to medium-sized cycloalkane carboxylic acids (FIG. 1D).

### BRIEF DESCRIPTION OF THE FIGURES

FIG. 1. Transannular C—H arylation via activation of methylene C—H bonds.

FIG. 2. Substrate scope of cyclopentane carboxylic acids.

FIG. 3. Transannular γ-C—H arylation.

FIG. 4. Transannular C—H arylation of cycloalkane carboxylic acids.

### SUMMARY OF THE INVENTION

β- and γ-C—H functionalizations of free carboxylic acids have been realized recently through the development of bifunctional ligands. However, γ-C—H functionalization of cyclic carboxylic acids remains a significant challenge due to the strain encountered in transannular C—H palladation. Herein reported is a class of ligands-quinuclidine-pyridones (QuinNuPyridones L1, L2) that enables transannular γ-methylene C—H arylation of small to medium sized cycloalkane carboxylic acids, with ring sizes ranging from cyclopentane to cyclooctane. Excellent γ-regioselectivity was observed in the presence of multiple β-C—H bonds. This advance marks an important step towards achieving molecular editing of saturated carbocycles: a class of scaffolds that are important in synthetic and medicinal chemistry. The utility of this protocol was demonstrated by two-step formal syntheses of a series of patented biologically active small molecules, prior syntheses of which required up to eleven steps.

### DETAILED DESCRIPTION OF THE INVENTION

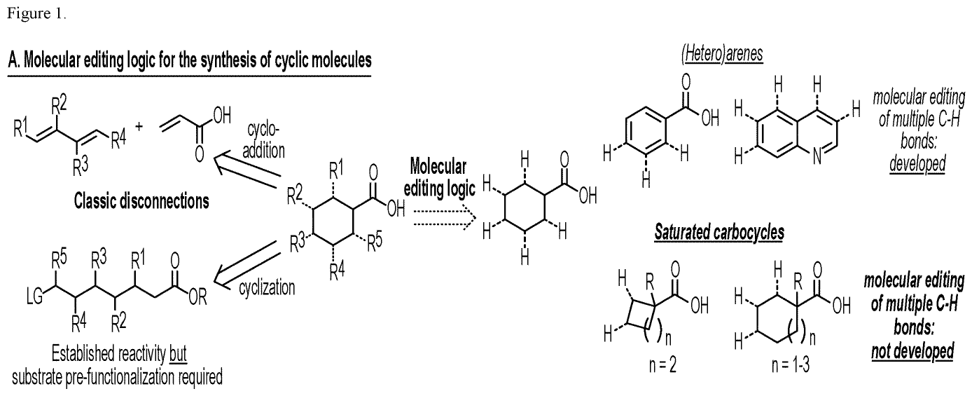

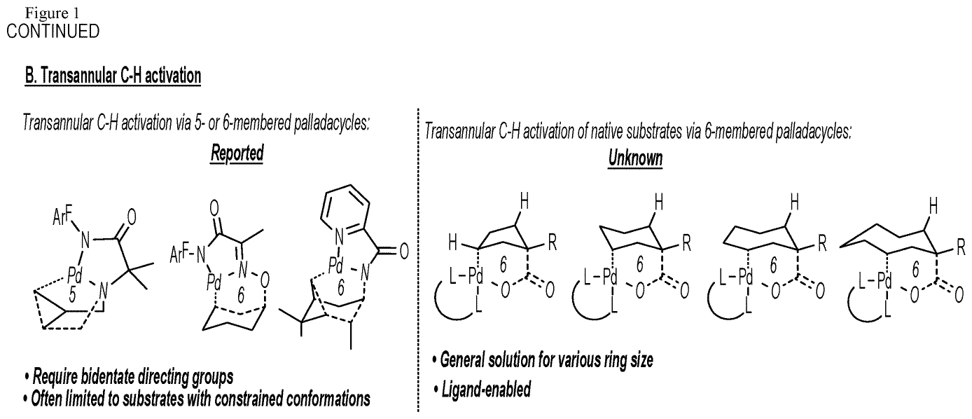

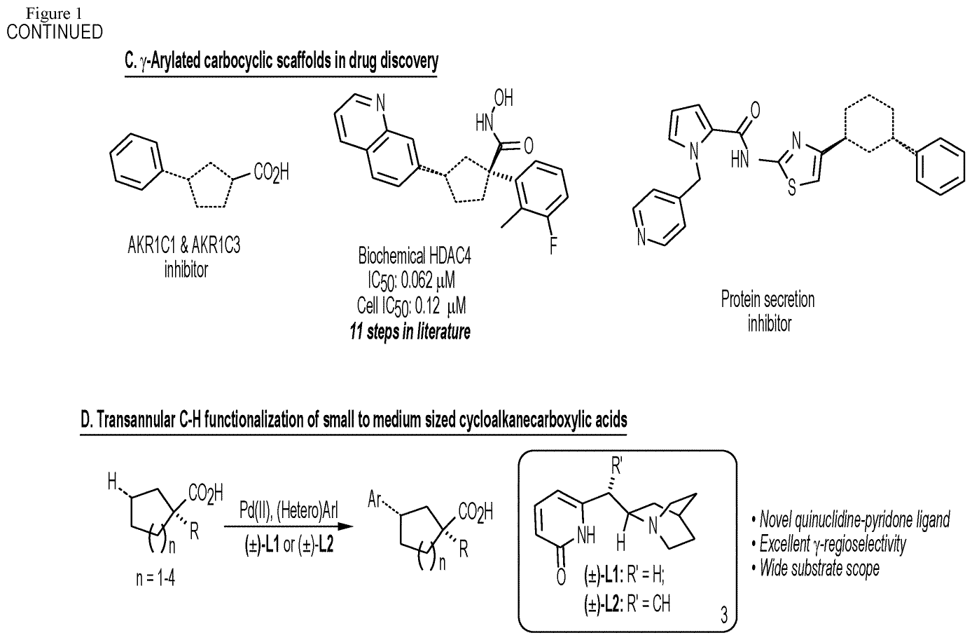

A quinuclidine-pyridone ligand enables the transannular γ-arylation of carboxylic acid substrates ranging from cyclopentane to cyclooctane rings. These transformations display excellent selectivity for transannular arylation of the γ-position in the presence of typically more reactive β-methyl and methylene C—H bonds^{6,34,35}, providing expedient access to a valuable family of compounds that previously necessitated lengthy synthetic sequences, sometimes exceeding ten steps³⁶. Notably, γ-arylated cycloalkane acids and acid derivatives often display important biological activity³⁶⁻⁴³, rendering this transformation especially valuable for medicinal chemistry (FIG. 1C). The development of this T-selective C—H arylation reaction also expands the capability of methylene C—H activation reactions from dehydrogenative transformations^{44,45}, H-D exchange⁴⁶, and intramolecular lactonizations³³, to intermolecular γ-arylations, opening another avenue for the generation of versatile synthetic intermediates for the preparation of complex, biologically active molecules.

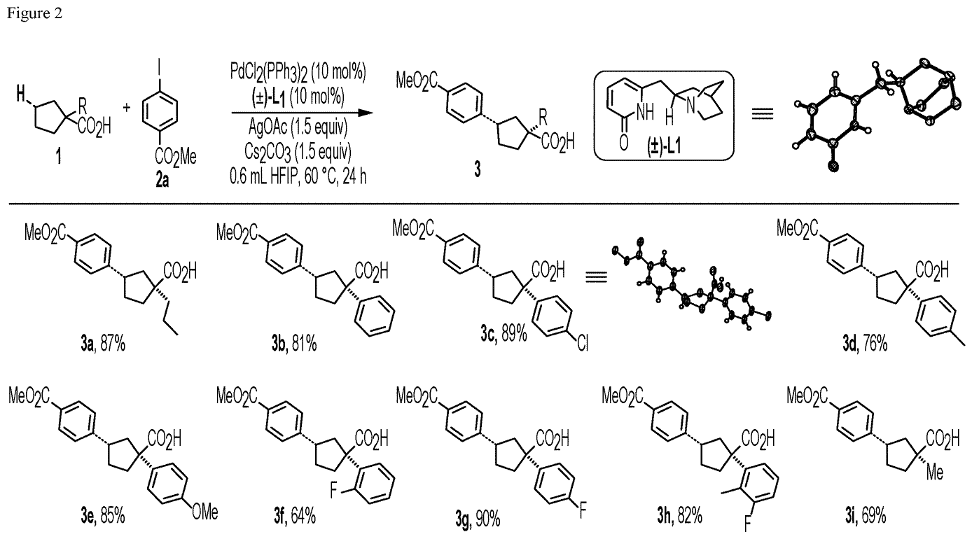

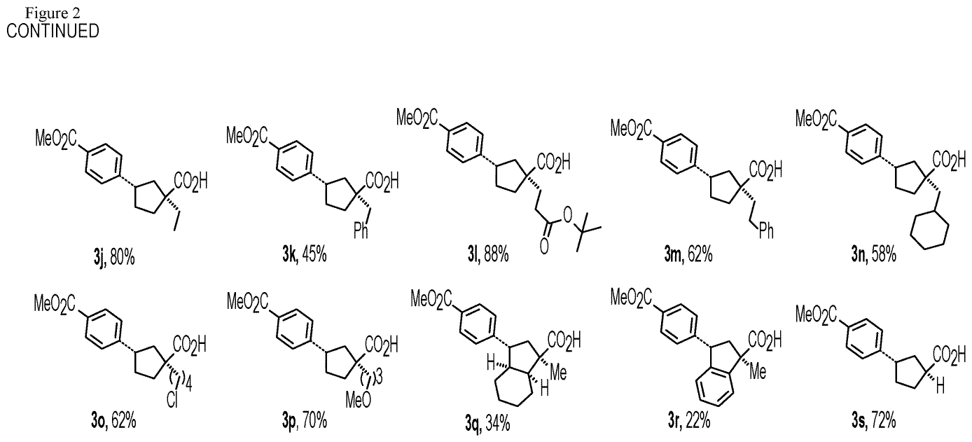

This investigation of transannular C—H arylation was initiated using α-propyl-cyclopentane carboxylic acid 1a as a model substate. Ligands were found to play a crucial role in this transformation (see table S1). While ligands known to promote β-C(sp³)-H activation, such as mono-protected aminoethyl phenyl thioether (MPAThio)⁴⁷, monoprotected amino acid (MPAA)^{48,49}, and monoprotected aminoethyl amine (MPAAm)⁵⁰ failed to provide any of the desired product, we observed that quinoline-pyridone ligands, which have been demonstrated to enable β- and γ-methylene C—H activation of free acids^{33,44,45}, afforded the desired γ-arylated product in 30% yield. Similar yields were also observed with a tertiary amine-pyridone ligand, confirming the importance of the pyridone moiety. It was hypothesized that replacing the tertiary amine in that scaffold with a quinuclidine might have a profound effect on the reactivity of the catalyst due to the special character of the quinuclidine motif, which is more Lewis basic and more rigid than typical tertiary amines⁵¹. Notably, it was found that quinuclidine-pyridone ligand L1 is uniquely effective in promoting the transannular γ-arylation, providing product 3a in 69% yield. The structure of L1 was confirmed by X-ray crystallography (FIG. 2). After further optimization of the reaction conditions (see tables S2-S7), the desired γ-arylated product was able to be obtained in 87% isolated yield.

The generality of the transannular C—H functionalization was investigated by varying the structure and ring size of the cycloalkane carboxylic acid and the identity of the (hetero)aryl iodide coupling partner. First examined was the substrate scope of α-quaternary cyclopentane carboxylic acids (FIG. 2). Substrates with α-aryl substituents, which contain γ-C(sp²)-H bonds that could compete with the desired site of functionalization, all provided the transannular γ-C(sp³)-H arylation products in good to excellent yields regardless of the electronic properties on the aryl ring (3b-3h). No γ-C(sp²)-H or β-C(sp³)-H arylation products were observed, demonstrating the exquisite regioselectivity of the reaction. X-ray crystallographic characterization of 3c confirmed that the aryl group is installed cis to the carboxylic acid directing group. Carboxylic acids with α-Me and α-Et substituents also performed well, affording the transannular γ-arylation products with 69% and 80% yield respectively (3i, 3j). Other α-alkyl substituents including those with additional functional groups, such as an ester, chloride, or ether substituent (3l, 3o-3p), were also compatible with this methodology. In addition, modest yields were obtainable with bicyclic systems such as 3q and 3r. Notably, transannular γ-arylation of α-hydrogen cyclopentane carboxylic acid also proceeded smoothly to give the target product 3s in good yield.

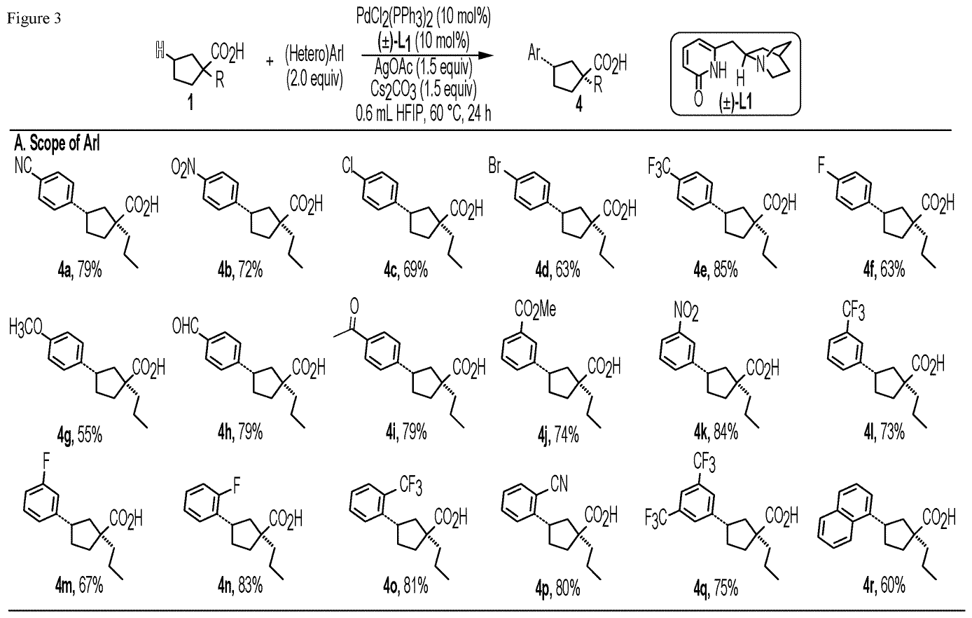

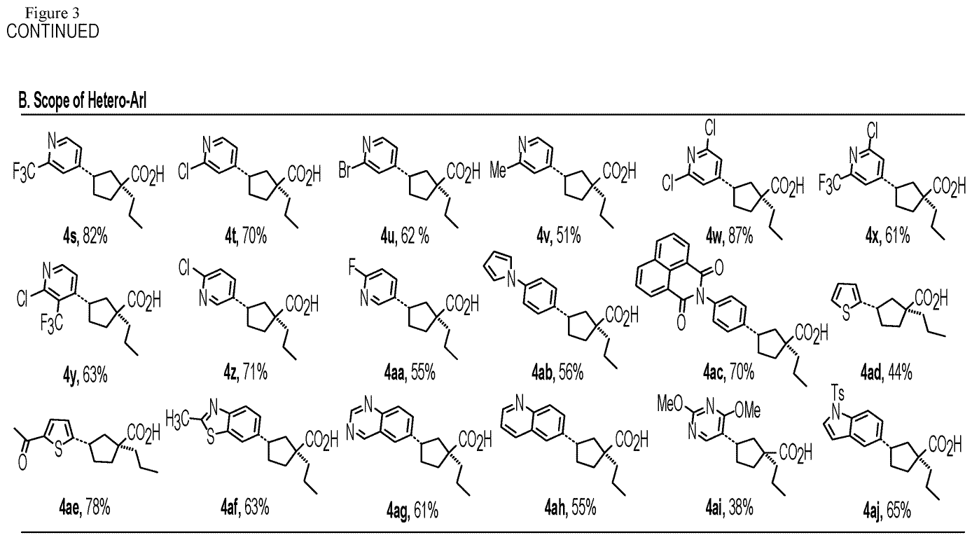

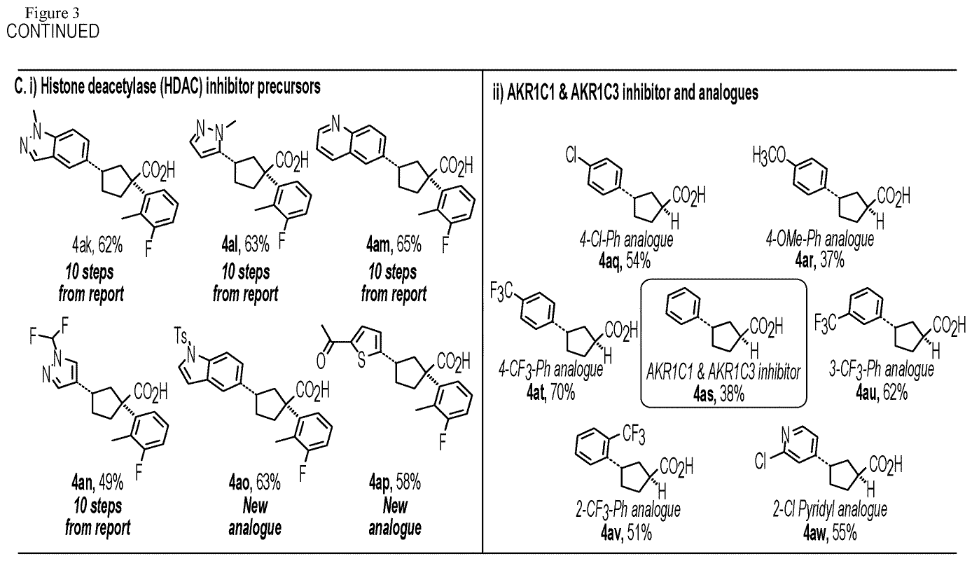

Next, examined was the scope of the ArI coupling partner using α-propyl cyclopentane carboxylic acid as a model substrate (FIG. 3A). A wide range of electronically varied para-substituted aryl iodides were compatible with this protocol, providing the transannular arylation products in 55% to 85% yield (4a-4i). In general, electron deficient aryl iodides gave slightly higher yields than those with electron donating groups (e.g., 4e vs 4g). Meta- and ortho-substituted aryl iodides also performed well in the reaction (4j-4p). Heteroarylation with heteroaryl iodides was then investigated (FIG. 3B). A variety of 2-substituted-4-iodopyridines reacted smoothly to provide the target products (4s-4v). 2,6- and 2,3-disubstituted-4-iodopyridines and 2-substituted-5-iodopyridines were also viable coupling partners for the transannular reaction, leading to moderate to excellent yields of the corresponding γ-arylated products (4w-4aa). Notably, thiophene, benzothiazole, quinazoline, quinoline, pyrimidine and indole based heteroaryl iodides were all successful coupling partners in the γ-arylation reaction, with the 2-acetyl-5-iodothiophene leading to the highest yield (4ae) (78%). 2,4-dimethoxy-5-iodopyrimidine only provided the product with 38% yield, presumably due to the strong electron donating effect of the dimethoxy group (4ai).

The great potential of this protocol for medicinal chemistry was subsequently demonstrated through the preparation of several reported histone deacetylase (HDAC) inhibitor precursors (FIG. 3C.-i), the synthesis of which previously required up to ten steps with very low overall yields³⁶. For example, a patented procedure details a 10-step synthesis of 4ak as a mixture of diastereomers at the benzylic position, which were subsequently separated by preparative HPLC to access the enantiopure compound. Employing this transannular γ-C—H arylation, racemic 4ak and its analogues 4al-4an can be easily accessed as single diastereomers in 49% to 65% yield in just one step from the commercially available α-aryl cyclopentane carboxylic acid. Notably, amidation of α-arylated cyclopentane carboxylic acid 4am affords potent histone deacetylase (HDAC) inhibitors with IC₅₀ values as low as 0.062 μm³⁶ (FIG. 1 C). Heterocyclic analogues were also prepared using indole and thiophene based aryl iodides (4ao-4ap). Inhibitors for AKR1C1 and AKR1C3 are promising scaffolds for the treatment of hormone-dependent cancers and other diseasess³⁷. Transannular C—H arylation of α-hydrogen containing cyclopentane carboxylic acid provides a one-step synthesis of reported AKR1C1 and AKR1C3 inhibitor 4as and a series of analogs (4aq-4aw) in 37% to 72% yield (FIG. 3C-ii). In contrast to the previously reported route, this transannular C—H activation chemistry provides diastereocontrol and allows for easy variation of the aryl substituent, facilitating further exploration of these compounds in medicinal chemistry.

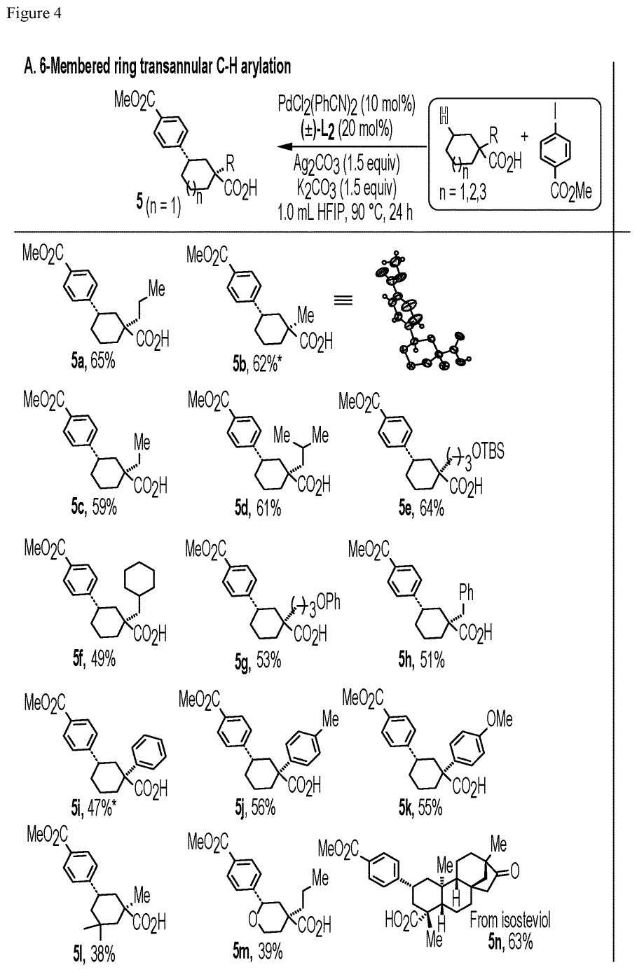

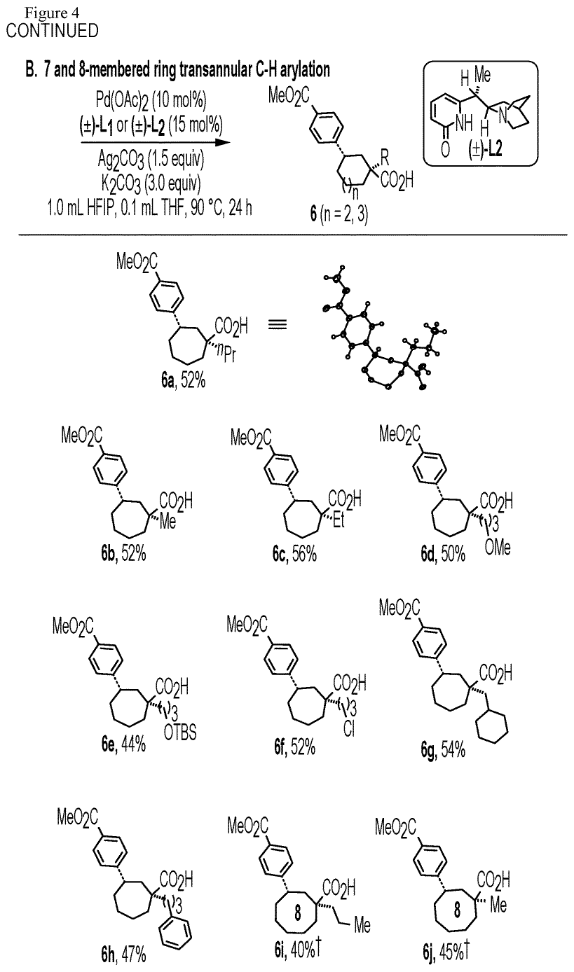

Next investigated was the transannular γ-C—H arylation of larger cycloalkane carboxylic acids (FIG. 4). Applying the optimal conditions identified for the arylation of cyclopentane carboxylic acids to α-propyl cyclohexane carboxylic acid only resulted in a 35% yield of the γ-arylated product. Fortunately, after a brief optimization of the ligands and the reaction conditions, it was found that L2 could provide the desired product (5a) in 65% isolated yield (see tables S8-S13 for details). Next, examined was the substrate scope of the transannular γ-arylations of cyclohexane carboxylic acids (FIG. 4A). Excellent regioselectivity was observed with substrates containing multiple potential reaction sites, such as α-methyl and α-ethyl cyclohexane carboxylic acids (5b-5c). The relative stereochemistry was confirmed by X-ray crystallography of 5b (FIG. 4A). α-Aryl substituted cyclohexane acids were also effective substrates (5i-5k). A substrate with geminal dimethyl substitution at the 7′-position was also reactive, albeit with a modest yield, possibly due to the 1,3-diaxial strain between the axial methyl substituent and the transannular palladacycle (51). Notably, transannular arylation of a tetrahydropyran-containing substrate (5m) proceeded smoothly, indicating that this methodology can be applied to the functionalization of saturated heterocycles. In addition, our protocol proved effective in complex settings such as late-stage functionalizations, as demonstrated through the arylation of the natural product isosteviol in 63% yield (5n). Transannular γ-C—H arylation of seven and eight membered cycloalkane carboxylic acids were also achieved, providing the corresponding products with moderate to good yields (6a-6j, FIG. 4B) (see tables S14-S17 for optimization details). The structure of 6a was confirmed by X-ray crystallography (FIG. 4B).

Thus, as described herein, transannular γ-methylene C—H arylation of cycloalkane carboxylic acids with QuinNuPyridone ligands (L1 and L2) has been achieved. A wide range of cycloalkane carboxylic acids with ring sizes ranging from five to eight were effective substrates in reactions with a diverse array of (hetero)aryl iodide coupling partners. Excellent regioselectivity was demonstrated with substrates containing multiple potential sites for C—H activation. Accordingly, these reactions provide general and straightforward access to γ-arylated cycloalkane acids, which are versatile synthetic building blocks, and in some cases, exhibit notable biological activity even without further elaboration. The utility of this protocol was demonstrated through the concise preparation of a series of biologically active compounds, previous syntheses of which required lengthy sequences. It is anticipated that this methodology will dramatically simplify the synthesis of γ-arylated cycloalkanes, helping to fuel the design and discovery of novel therapeutics.

### REFERENCES

1. Veber, D. F., Johnson, S. R., Cheng, H.-Y., Smith, B. R., Ward, K. W. & Kopple, K. D. Molecular Properties that Influence the Oral Bioavailability of Drug Candidates. J. Med. Chem. 45, 2615-2623 (2002).
2. Fang, Z., Song, Y., Zhan, P., Zhang, Q. & Liu, X. Conformational restriction: an effective tactic in ‘follow-on’-based drug discovery. Fut. Med. Chem. 6, 885-901 (2014).
3. Shearer, J., Castro, J. L., Lawson, A. D. G., MacCoss, M. & Taylor, R. D. Rings in Clinical Trials and Drugs: Present and Future. J. Med. Chem. 65, 8699-8712 (2022).
4. Nicolaou, K. C. & Sorensen, E. J. Classics in Total Synthesis: Targets, Strategies, Methods. (Wiley, 1996).
5. Corey, E. J. & Cheng, X.-M. The Logic of Chemical Synthesis. (Wiley, 1989).
6. Ma, S. Handbook of Cyclization Reactions. (Wiley, 2009).
7. Brill, Z. G., Condakes, M. L., Ting, C. P. & Maimone, T. J. Navigating the Chiral Pool in the Total Synthesis of Complex Terpene Natural Products. Chem. Rev. 117, 11753-11795 (2017).
8. Xia, G., Weng, J., Liu, L., Verma, P., Li, Z. & Yu, J.-Q. Reversing conventional site-selectivity in C(sp³)-H bond activation. Nat. Chem. 11, 571-577 (2019).
9. Leow, D., Li, G., Mei, T.-S. & Yu, J.-Q. Activation of remote meta-C—H bonds assisted by an end-on template. Nature 486, 518-522 (2012).
10. Tang, R.-Y., Li, G. & Yu, J.-Q. Conformation-induced remote meta-C—H activation of amines. Nature 507, 215-220 (2014).
11. Zhang, Z., Tanaka, K. & Yu, J.-Q. Remote site-selective C—H activation directed by a catalytic bifunctional template. Nature 543, 538-542 (2017).
12. Lam, N. Y. S., Fan, Z., Wu, K., Park, H. S., Shim, S. Y., Strassfeld, D. A. & Yu, J.-Q. Empirical Guidelines for the Development of Remote Directing Templates through Quantitative and Experimental Analyses. J. Am. Chem. Soc. 144, 2793-2803 (2022).
13. Fan, Z., Chen, X., Tanka, K., Park, H. S., Lam, N. Y. S., Wong, J. J., Houk, K. N. & J.-Q. Yu, Molecular editing of aza-arene C—H bonds by distance, geometry and chirality. Nature 610, 87-93 (2022).
14. Meng, G., Lam, N. Y. S., Lucas, E. L., Sain-Denis, T. G., Verma, P., Checkshin, N. & Yu, J.-Q. Achieving Site-Selectivity for C—H Activation Processes Based on Distance and Geometry: A Carpenter's Approach. J. Am. Chem. Soc. 142, 10571-10591 (2020).
15. Lovering, F., Bikker, J. & Humblet, C. Escape from Flatland: Increasing Saturation as an Approach to Improving Clinical Success. J. Med. Chem. 52, 6752-6756 (2009).
16. Zaitsev, V. G., Shabashov, D. & Daugulis, O. Highly Regioselective Arylation of sp³ C—H Bonds Catalyzed by Palladium Acetate. J. Am. Chem. Soc. 127, 13154-13155 (2005).
17. Banerjee, A., Sarkar, S. & Patel, B. K. C—H functionalization of cycloalkanes. Org. & Biomol. Chem. 15, 505-530 (2017).
18. Andra, M. S., Schifferer, L., Pollok, C. H., Merten, C., Gooßen, L. J. & Yu, J.-Q. Enantio- and Diastereoswitchable C—H Arylation of Methylene Groups in Cycloalkanes. Chem. Eur. J. 25, 8503-8507 (2019).
19. He, G. & Chen, G. A Practical Strategy for the Structural Diversification of Aliphatic Scaffolds through the Palladium-Catalyzed Picolinamide-Directed Remote Functionalization of Unactivated C(sp3)-H Bonds. Angew. Chem. Int. Ed. 50, 5192-5196 (2011).
20. Cui, W., Chen, S., Wu, J., Zhao, X., Hu, W. & Wang, H. Palladium-Catalyzed Remote C(sp³)-H Arylation of 3-Pinanamine. Org. Lett. 16, 4288-4291 (2014).
21. Seki, A., Takahashi, Y. & Miyake, T. Synthesis of cis-3-arylated cycloalkylamines through palladium-catalyzed methylene sp³ carbon-hydrogen-bond activation. Tetrahedron Lett. 55, 2838-2841 (2014).
22. Wu, Y., Chen, Y., Liu, T., Eastgate, M. D. & Yu, J. Q. Pd-Catalyzed 7-C(sp³)-H Arylation of Free Amines Using a Transient Directing Group. J. Am. Chem. Soc. 138, 14554-14557 (2016).
23. Topczewski, J. J., Cabrera, P. J., Saper, N. I. & Sanford, M. S. Palladium-catalysed transannular C—H functionalization of alicyclic amines. Nature, 531, 220-224 (2016).
24. Xu, Y., Young, M. C., Wang, C., Magness, D. M. & Dong, G. Catalytic C(sp3)-H Arylation of Free Primary Amines with an exo Directing Group Generated in Situ. Angew. Chem. Int. Ed. 55, 9084-9087 (2016).
25. Van Steijvoort, B. F., Kaval, N., Kulago, A. A. & Maes, B. U. W. Remote Functionalization: Palladium-Catalyzed C5(sp3)-H Arylation of 1-Boc-3-Aminopiperidine through the Use of a Bidentate Directing Group. ACS Catal. 6, 4486-4490 (2016).
26. Zhao, J., Zhao, X., Cao, P., Liu, J. & Wu, B. Polycyclic Azetidines and Pyrrolidines via Palladium-Catalyzed Intramolecular Amination of Unactivated C(sp³)-H Bonds. Org. Lett. 19, 4880-4883 (2017).
27. Coomber, C. E., Benhamou, L., Bucar, D.-K., Smith, P. D., Porter, M. J. & Sheppard, T. D. Silver-Free Palladium-Catalyzed C(sp³)-H Arylation of Saturated Bicyclic Amine Scaffolds. J Org. Chem. 83, 2495-2503 (2018).
28. Cabrera, P. J., Lee, M. & Sanford, M. S. Second-Generation Palladium Catalyst System for Transannular C—H Functionalization of Azabicycloalkanes. J. Am. Chem. Soc. 140, 5599-5606 (2018).
29. Chen, Y. Q., Wang, Z., Wu, Y., Wisniewski, S. R., Qiao, J. X., Ewing, W. R., Eastgate, M. D. & Yu, J. Q. Overcoming the Limitations of γ- and δ-C—H Arylation of Amines through Ligand Development. J. Am. Chem. Soc. 140, 17884-17894 (2018).
30. Landge, V. G., Parveen, A., Nandakumar, A. & Balaraman, E. Pd(II)—Catalyzed Gamma-C(sp3)-H Alkynylation of Amides: Selective Functionalization of R Chains of Amides R¹C(O)NHR. Chem. Commun. 54, 7483-7486 (2018).
31. Aguilera, E. Y. & Sanford, M. S. Model Complexes for the Palladium-Catalyzed Transannular C—H Functionalization of Alicyclic Amines. Organometallics, 38, 138-142 (2019).
32. Li, Z., Dechantsreiter, M. & Dandapani, S. Systematic Investigation of the Scope of Transannular C—H Heteroarylation of Cyclic Secondary Amines for Synthetic Application in Medicinal Chemistry. J Org. Chem. 85, 6747-6760 (2020).
33. Chan, H. S. S., Yang, J. & Yu, J.-Q. Catalyst-controlled site-selective methylene C—H lactonization of dicarboxylic acids, Science 376, 1481-1487 (2022).
34. Liu, B., Romine, A. M., Rubel, C. Z., Engle, K. M. & Shi, B.-F. Transition-Metal-Catalyzed, Coordination-Assisted Functionalization of Nonactivated C(sp³)-H Bonds. Chem. Rev. 121, 14957-15074 (2021).
35. Lucas, E. L., Lam, N. Y. S., Zhuang, Z., Chan, H. S. S., Strassfeld, D. A. & Yu, J.-Q. Palladium-Catalyzed Enantioselective p-C(sp³)-H Addition, Activation Reactions of Aliphatic Acids: A Retrosynthetic Surrogate for Enolate Alkylation and Conjugate Additions. Acc. Chem. Res. 55, 537-550 (2022).
36. Dominguez, C., Munoz-Sanjuan, I., Maillard, M., Vater, H. D., Jarvis, R. E., Luckhurst, C. A., Buerli, R. W., Wishart, G. & Stott, A. I. Histone deacetylase inhibitors for treatment of neurodegenerative diseases. WO 2014/159224 A1, Oct. 2, 2014.
37. Stefane, B., Brozic, P., Vehovc, M., Rizner, T. L. & Gobec, S. New cyclopentane derivatives as inhibitors of steroid metabolizing enzymes AKR1C1 and AKR1C3, Eur. J. Med. Chem. 44, 2563-2571 (2009).
38. Haydar, S. N., Li, L., Bures, M. G., Rai, R., Bannen, L. & Walker, M. 5-Membered heteroaryl carboxamide compounds for treatment of HBV, WO 2020/086533 A1, Apr. 30, 2020.
39. Chen, H., Safina, B., Shore, D., Terrett, J., Villemure, E., Volgraf, M., Ward, S., Lu, A., Larouche-Gauthier, R., Beaumier, F., Dery, M., Constantineau-Forget, L., Grand-Maitre, C. & Lepissier, L. Oxadiazole transient receptor potential channel inhibitors, WO 2018/162607 A1, Sep. 13, 2018.
40. Chobanian, H., Demong, D., Guo, Y., Hu, Z., Miller, M., Pio, B., Plummer, C. W., Xiao, D., Yang, C. & Zhang, R. Antidiabetic compounds, WO 2015/119899 A1, Aug. 13, 2015.
41. McMinn, D. & Rao, M. Thiazole derivatives as protein secretion inhibitors, WO 2020/176863 A1, Sep. 3, 2020.
42. Li, L. & Zhong, M. Inhibitors of HCV NS5A, WO 2010/065681 A1, Jun. 10, 2010.
43. Wager, T. T., Pettersen, B. A., Schmidt, A. W., Spracklin, D. K., Mente, S., Butler, T. W., Howard, H., Lettiere, Jr., D. J., Rubitski, D. M., Wong, D. F., Nedza, F. M., Nelson, F. R., Rollema, H., Raggon, J. W., Aubrecht, J., Freeman, J. K., Marcek, J. M., Cianfrogna, J., Cooki, K. W., James, L. C., Chatman, L. A., Iredale, P. A., Banker, M. J., Homiski, M. L., Munzner, J. B. & Chandrasekaran, R. Y. Discovery of Two Clinical Histamine H₃ Receptor Antagonists: trans-N-Ethyl-3-fluoro-3-[3-fluoro-4-(pyrrolidinylmethyl)-phenyl]cyclobutanecarboxamide (PF-03654746) and trans-3-Fluoro-3-[3-fluoro-4-(pyrrolidin-1-ylmethyl)phenyl]-N-(2-methylpropyl)cyclobutanecarboxamide (PF-03654764). J. Med. Chem. 54, 7602-7620 (2011).
44. Wang, Z., Hu, L., Chekshin, N., Zhuang, Z., Qian, S., Qiao, J. X. & Yu, J.-Q. Ligand-controlled divergent dehydrogenative reactions of carboxylic acids via C—H activation. Science. 374, 1281-1285 (2021).
45. Sheng, T., Zhuang, Z., Wang, Z., Hu, L., Herron, A. N., Qiao, J. X. & Yu, J.-Q. One-Step Synthesis of β-Alkylidene-γ-lactones via Ligand-Enabled β,γ-Dehydrogenation of Aliphatic Acids, J. Am. Chem. Soc. 144, 12924-12933 (2022).
46. Uttry, A., Mal, S. & van Gemmeren, M. Late-Stage β-C(sp³)-H Deuteration of Carboxylic Acids. J. Am. Chem. Soc. 143, 10895-10901 (2021).
47. Zhuang, Z., Herron, A. N. & Yu, J. Q. Synthesis of Cyclic Anhydrides via Ligand-Enabled C—H Carbonylation of Simple Aliphatic Acids. Angew. Chem. Int. Ed. 60, 16382-16387 (2021).
48. Hu, L., Shen, P., Shao, Q., Hong, K., Qiao, J. X. & Yu. J. Q. Pd(II)-catalyzed enantioselective C(sp³)-H activation/cross-coupling reactions of free carboxylic acids. Angew. Chem., Int. Ed. 58, 2134-2138 (2019).
49. Zhuang, Z., Herron, A. N., Liu, S. & Yu, J. Q. Rapid Construction of Tetralin, Chromane, and Indane Motifs via Cyclative C—H/C—H Coupling: Four-Step Total Synthesis of (±)-Russujaponol F. J. Am. Chem. Soc. 143, 687-692 (2021).
50. Shen, P., Hu, L., Shao, Q., Hong, K. & Yu. J. Q. Pd(II)-catalyzed enantioselective C(sp³)-H arylation of free carboxylic acids. J. Am. Chem. Soc. 140, 6545-6549 (2018).
51. Hamama, W. S., El-Magid, O. M. A. & Zoorob, H. H. Chemistry of Quinuclidines as Nitrogen Bicyclic Bridged-ring Structures. J. Heterocyclic Chem. 43, 1397-1420 (2006).
52. Chen, G., Gong, W., Zhuang, Z., Andra, M. S., Chen, Y.-Q., Hong, X., Yang, Y.-F., Liu, T., Houk, K. N. & Yu, J.-Q. Ligand-accelerated enantioselective methylene C(sp³)-H bond activation. Science 353, 1023-1027 (2016).
53. Chen, G., Zhuang, Z., Li, G.-C., Saint-Denis, T. G., Hsiao, Y., Joe, C. L. & Yu, J.-Q. Ligand-Enabled β-C—H Arylation of α-Amino Acids Without Installing Exogenous Directing Groups. Angew. Chem., Int. Ed. 56, 1506-1509 (2017).
54. Zhuang, Z., Yu, C.-B., Chen, G., Wu, Q.-F., Hsiao, Y., Joe, C. L., Qiao, J. X., Poss, M. A. & Yu, J.-Q. Ligand-Enabled β-C(sp³)-H Olefination of Free Carboxylic Acids. J. Am. Chem. Soc. 140, 10363-10367 (2018).
55. Strassfeld, D. A., Chen, C.-Y., Park, H. S., Phan, J. & Yu, J.-Q. Manuscript in preparation.
56. Li, C.-J. Cross-Dehydrogenative Coupling (CDC): Exploring C—C Bond Formations beyond Functional Group Transformations. Acc. Chem. Res. 42, 335-344 (2009).
57. Wang, P., Verma, P., Xia, G., Shi, J., Qiao, J. X., Tao, S., Cheng, P. T. W., Poss, M. A., Farmer, M. E., Yeung, K.-S. & Yu, J.-Q. Ligand-accelerated non-directed C—H functionalization of arenes. Nature 551, 489-493 (2017).

### EMBODIMENTS

Embodiment 1. A method of transannular γ-methylene C(sp³)-H arylation of cycloalkane carboxylic acids, comprising treating a cycloalkyl carboxylic acid with an aryl or heteroaryl iodide in the presence of a quinuclidine-pyridone and a Pd source.

Embodiment 2. The method of Embodiment 1, wherein the method of γ-methylene C(sp³)-H arylation of cycloalkane carboxylic acids occurs according to the following reaction scheme:

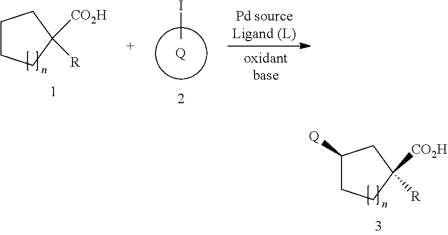

wherein:
Q is Ar or Het;
   Ar is (C₆-C₁₀)aryl, optionally substituted with one or more R^{a};
   Het is unsaturated or partially unsaturated (C₅-C₈)heteroaryl, optionally substituted with one or more R^{a};
R is H or selected from the group consisting of (C₁-C₆)alkyl, (C₂₋₆)alkenyl, (C₂₋₆)alkynyl, (C₁-C₆)heteroalkyl, hetero (C₂₋₆)alkenyl, hetero (C₂₋₆)alkynyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, each independently and optionally substituted with one or more R^{a};
   each R^{a} is independently selected from —C(═O)O(C₁-C₆)alkyl, CN, NO₂, halo, OH, —C(═O)H, NH₂, (C₁-C₆)alkyl, halo (C₁-C₆)alkyl, (C₁-C₆)heteroalkyl, halo (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl; and
n is 1, 2, 3, or 4;
including enantiomers, scalemic or racemic mixtures, and pharmaceutically acceptable salts thereof.

Embodiment 3. The method of Embodiment 2, wherein n is 1, Ligand (L) is selected from the group consisting of:

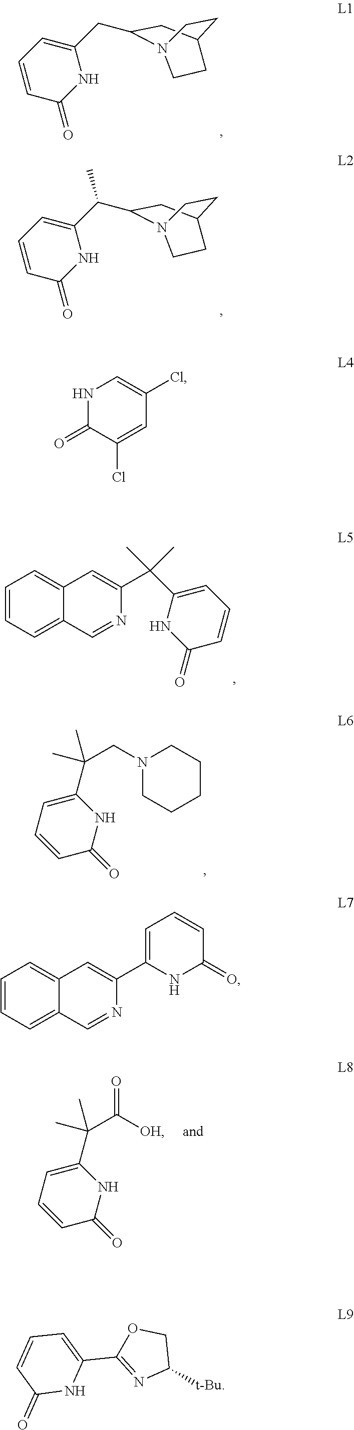

Embodiment 4. The method of Embodiment 2, wherein n is 2, Ligand (L) is selected from the group consisting of:

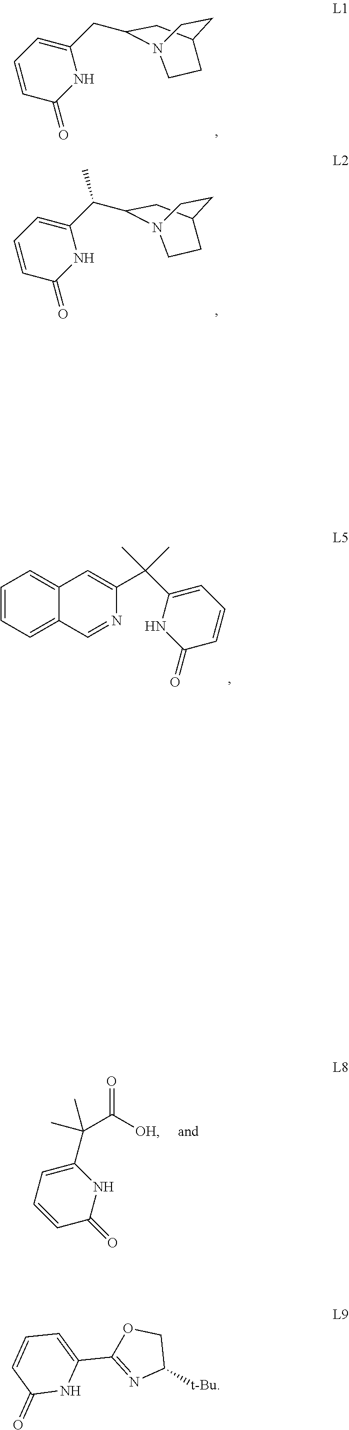

Embodiment 5. The method of Embodiment 2, wherein n is 3, Ligand (L) is L2.

Embodiment 6. The method of Embodiment 2, wherein n is 4, Ligand (L) is L1 or L2.

Embodiment 7. The method of either Embodiment 2 or Embodiment 3, wherein n is 1.

Embodiment 8. The method of either Embodiment 2 or Embodiment 4, wherein n is 2.

Embodiment 9. The method of either Embodiment 2 or Embodiment 5, wherein n is 3.

Embodiment 10. The method of either Embodiment 2 or Embodiment 6, wherein n is 4.

Embodiment 11. The method of any one of Embodiments 2-10, wherein Q is Ar optionally substituted with one or more R^{a}.

Embodiment 12. The method of Embodiment 11, wherein Ar is Ph optionally substituted with one or more R^{a}.

Embodiment 13. The method of Embodiment 12, wherein R^{a} is —C(═O)Me.

Embodiment 14. The method of Embodiment 12, wherein R^{a} is halo.

Embodiment 15. The method of Embodiment 14, wherein halo is Cl.

Embodiment 16. The method of Embodiment 14, wherein halo is F.

Embodiment 17. The method of Embodiment 14, wherein halo is Br.

Embodiment 18. The method of Embodiment 12, wherein R^{a} is CN.

Embodiment 19. The method of Embodiment 12, wherein R^{a} is NO₂.

Embodiment 20. The method of Embodiment 12, wherein R^{a} is (C₁-C₆)alkyl.

Embodiment 21. The method of Embodiment 12, wherein R^{a} is halo (C₁-C₆)alkyl.

Embodiment 22. The method of Embodiment 21, wherein halo (C₁-C₆)alkyl is —CF₃.

Embodiment 23. The method of Embodiment 12, wherein R^{a} is (C₁-C₆)heteroalkyl.

Embodiment 24. The method of Embodiment 23, wherein (C₁-C₆)heteroalkyl is —OMe.

Embodiment 25. The method of Embodiment 12, wherein R^{a} is —C(═O)(C₁-C₆)alkyl.

Embodiment 26. The method of Embodiment 12, wherein R^{a} is —C(═O)H.

Embodiment 27. The method of Embodiment 12, wherein R^{a} is —C(═O)OMe.

Embodiment 28. The method of any one of Embodiments 2-10, wherein Q is Het optionally substituted with one or more R^{a}.

Embodiment 29. The method of Embodiment 28, wherein Het is pyridinyl.

Embodiment 30. The method of Embodiment 28, wherein Het is thiophenyl.

Embodiment 31. The method of Embodiment 28, wherein Het is pyrimidinyl.

Embodiment 32. The method of Embodiment 28, wherein Het is 1H-indolyl.

Embodiment 33. The method of Embodiment 28, wherein Het is quinolinyl.

Embodiment 34. The method of Embodiment 28, wherein Het is quinazolinyl.

Embodiment 35. The method of Embodiment 28, wherein Het is benzo[d]thiazolyl.

Embodiment 36. The method of Embodiment 28, wherein Het is indazolyl.

Embodiment 37. The method of Embodiment 28, wherein Het is pyrazolyl.

Embodiment 38. The method of any one of Embodiments 28-37, wherein R^{a} is —C(═O)Ome.

Embodiment 39. The method of any one of Embodiments 28-37, wherein R^{a} is halo.

Embodiment 40. The method of Embodiment 39, wherein halo is C₁.

Embodiment 41. The method of Embodiment 39, wherein halo is F.

Embodiment 42. The method of Embodiment 39, wherein halo is Br.

Embodiment 43. The method of any one of Embodiments 28-37, wherein R^{a} is —CF₃.

Embodiment 44. The method of any one of Embodiments 28-37, wherein R^{a} is Me.

Embodiment 45. The method of any one of Embodiments 28-37, wherein R^{a} is —C(═O)H.

Embodiment 46. The method of any one of Embodiments 28-37, wherein R^{a} is —C(═O)Me.

Embodiment 47. The method of any one of Embodiments 28-37, wherein R^{a} is —OMe.

Embodiment 48. The method of any one of Embodiments 28-37, wherein R^{a} is -Ts.

Embodiment 49. The method of any one of Embodiments 28-48, wherein a second R^{a} is present.

Embodiment 50. The method of Embodiment 49, wherein the second R^{a} is —OMe.

Embodiment 51. The method of Embodiment 49, wherein the second R^{a} is halo.

Embodiment 52. The method of Embodiment 49, wherein the second R^{a} is —CF₃.

Embodiment 53. The method of any one of Embodiments 2-52, wherein R is (C₁-C₆)alkyl.

Embodiment 54. The method of Embodiment 53, wherein R is (C₁-C₆)alkyl is Pr.

Embodiment 55. The method of Embodiment 53, wherein R is (C₁-C₆)alkyl is Me.

Embodiment 56. The method of Embodiment 53, wherein R is (C₁-C₆)alkyl is Et.

Embodiment 57. The method of any one of Embodiments 2-52, wherein R is (C₁-C₆)alkyl (C₃-C₇)cycloalkyl.

Embodiment 58. The method of any one of Embodiments 2-52, wherein R is (C₁-C₆)alkyl (C₆-C₁₀)aryl.

Embodiment 59. The method of any one of Embodiments 2-52, wherein R is (C₁-C₆)heteroalkyl.

Embodiment 60. The method of any one of Embodiments 2-52, wherein R is halo (C₁-C₆)alkyl.

Embodiment 61. The method of any one of Embodiments 2-52, wherein R is (C₁-C₆)alkyl-OTBS.

Embodiment 62. The method of any one of Embodiments 2-61, wherein L is a quinuclidine-pyridone.

Embodiment 63. The method of any one of Embodiments 2-4, 6-8, and 10-62, wherein L is L1.

Embodiment 64. The method of any one of Embodiments 2-62, wherein L is L2.

Embodiment 65. The method of any one of Embodiments 2-64, wherein the Pd source is Pd(OAc)₂.

Embodiment 66. The method of any one of Embodiments 2-64, wherein the Pd source is PdCl₂allyl₂.

Embodiment 67. The method of any one of Embodiments 2-64, wherein the Pd source is Pd(TFA)₂.

Embodiment 68. The method of any one of Embodiments 2-64, wherein the Pd source is PdCl₂(PhCN)₂.

Embodiment 69. The method of any one of Embodiments 2-64, wherein the Pd source is PdCl₂(ethylenediamine).

Embodiment 70. The method of any one of Embodiments 2-64, wherein the Pd source is PdCl₂(PPh₃)₂.

Embodiment 71. The method of any one of Embodiments 2-64, wherein the Pd source is Pd(dba)₃.

Embodiment 72. The method of any one of Embodiments 2-64, wherein the Pd source is PdCl₂(dppf).

Embodiment 73. The method of any one of Embodiments 2-72, wherein the oxidant is AgOAc.

Embodiment 74. The method of any one of Embodiments 2-72, wherein the oxidant is Ag₂CO₃.

Embodiment 75. The method of any one of Embodiments 2-72, wherein the oxidant is Ag₂O.

Embodiment 76. The method of any one of Embodiments 2-72, wherein the oxidant is AgNO₃.

Embodiment 77. The method of any one of Embodiments 2-72, wherein the oxidant is AgPO₄.

Embodiment 78. The method of any one of Embodiments 2-72, wherein the oxidant is AgTFA.

Embodiment 79. The method of any one of Embodiments 2-72, wherein the oxidant is Na₂CO₃.1.5H₂O₂.

Embodiment 80. The method of any one of Embodiments 2-79, wherein the base is Na₂CO₃.

Embodiment 81. The method of any one of Embodiments 2-79, wherein the base is Li₂CO₃.

Embodiment 82. The method of any one of Embodiments 2-79, wherein the base is K₂CO₃.

Embodiment 83. The method of any one of Embodiments 2-79, wherein the base is Cs₂CO₃.

Embodiment 84. The method of any one of Embodiments 2-79, wherein the base is NaHCO₃.

Embodiment 85. The method of any one of Embodiments 2-79, wherein the base is K₂HPO₄.

Embodiment 86. The method of any one of Embodiments 2-79, wherein the base is KH₂PO₄.

Embodiment 87. The method of any one of Embodiments 2-79, wherein the base is Na₃PO₄.

Embodiment 88. The method of any one of Embodiments 2-79, wherein the base is Na₂HPO₄·7H₂O.

Embodiment 89. The method of any one of Embodiments 2-79, wherein the base is t-BuOK.

Embodiment 90. The method of any one of Embodiments 2-79, wherein the base is CH₃CO₂Na.

Embodiment 91. The method of any one of Embodiments 2-79, wherein the base is Na₂SO₃.

Embodiment 92. The method of any one of Embodiments 2-79, wherein the base is Li₃PO₄.

Embodiment 93. The method of any one of Embodiments 2-79, wherein the base is KF.

Embodiment 94. The method of any one of Embodiments 2-79, wherein the base is t-AmONa.

Embodiment 95. The method of any one of Embodiments 2-79, wherein the base is HCO₂Na.

Embodiment 96. The method of any one of Embodiments 2-79, wherein the base is HCO₂K.

Embodiment 97. The method of any one of Embodiments 2-79, wherein the base is NaTFA.

Embodiment 98. The method of any one of Embodiments 2-79, wherein the base is CH₃CO₂Na.

Embodiment 99. The method of any one of Embodiments 2-79, wherein the base is CH₃CO₂K.

Embodiment 100. The method of any one of Embodiments 2-99, wherein the solvent is HFIP.

Embodiment 101. The method of any one of Embodiments 2-99, wherein the solvent is toluene.

Embodiment 102. The method of any one of Embodiments 2-99, wherein the solvent is dioxane.

Embodiment 103. The method of any one of Embodiments 2-99, wherein the solvent is t-amylol.

Embodiment 104. The method of any one of Embodiments 2-99, wherein the solvent is DMF.

Embodiment 105. The method of any one of Embodiments 2-99, wherein the solvent is MeCN.

Embodiment 106. The method of any one of Embodiments 2-99, wherein the solvent is THF.

Embodiment 107. The method of any one of Embodiments 2-99, wherein the solvent is CHCl₃.

Embodiment 108. The method of any one of Embodiments 2-107, wherein the reaction temperature is between approximately 40-120° C.

Embodiment 109. The method of any one of Embodiments 2-107, wherein the reaction temperature is between approximately 50-90° C.

Embodiment 110. The method of any one of Embodiments 2-107, wherein the reaction temperature is between approximately 55-65° C.

Embodiment 111. The method of any one of Embodiments 2-110, wherein the solvent volume is approximately 0.3-1.5 mL.

Embodiment 112. The method of any one of Embodiments 2-111, wherein the equivalents of aryl or heteroaryl iodide compared to carboxylic acid substrate is approximately between 1.5 and 2.5.

Embodiment 113. The method of any one of Embodiments 2-112, wherein the equivalents of aryl or heteroaryl iodide compared to carboxylic acid substrate is approximately 2.0.

Embodiment 114. The method of any one of Embodiments 2-113, wherein the equivalents of oxidant compared to carboxylic acid substrate is approximately between 0.5 and 2.0.

Embodiment 115. The method of any one of Embodiments 2-114, wherein the equivalents of oxidant compared to carboxylic acid substrate is approximately 1.5.

Embodiment 116. The method of any one of Embodiments 2-115, wherein the equivalents of base compared to carboxylic acid substrate is approximately between 0.5 and 2.0.

Embodiment 117. The method of any one of Embodiments 2-116, wherein the equivalents of base compared to carboxylic acid substrate is approximately 1.5.

Embodiment 118. The method of any one of Embodiments 2-117, wherein the mol % of Pd source is approximately between 5-15%.

Embodiment 119. The method of any one of Embodiments 2-118, wherein the mol % of Pd source is approximately 10%.

Embodiment 120. The method of any one of Embodiments 2-119, wherein the mol % of Ligand is approximately between 5-25%.

Embodiment 121. The method of any one of Embodiments 2-120, wherein the mol % of Ligand is approximately 20%.

Embodiment 122. The method of Embodiment 2, wherein the method of γ-methylene C(sp³)-H arylation of cycloalkane carboxylic acids occurs according to the following reaction scheme:

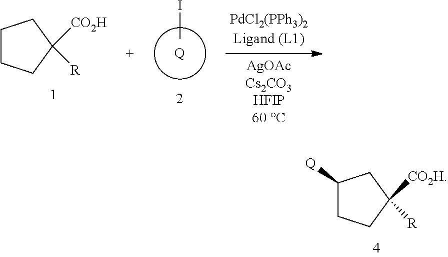

Embodiment 123. The method of Embodiment 122, wherein the product compound 4 is cis-1-(3-fluoro-2-methylphenyl)-3-(1-methyl-1H-indazol-5-yl)cyclopentane-1-carboxylic acid.

Embodiment 124. The method of Embodiment 122, wherein the product compound 4 is cis-1-(3-fluoro-2-methylphenyl)-3-(1-methyl-1H-pyrazol-5-yl)cyclopentane-1-carboxylic acid.

Embodiment 125. The method of Embodiment 122, wherein the product compound 4 is cis-1-(3-fluoro-2-methylphenyl)-3-(quinolin-6-yl)cyclopentane-1-carboxylic acid.

Embodiment 126. The method of Embodiment 122, wherein the product compound 4 is cis-3-(1-(difluoromethyl)-1H-pyrazol-4-yl)-1-(3-fluoro-2-methylphenyl)cyclopentane-1-carboxylic acid.

Embodiment 127. The method of Embodiment 122, wherein the product compound 4 is cis-1-(3-fluoro-2-methylphenyl)-3-(1-tosyl-1H-indol-5-yl)cyclopentane-1-carboxylic acid.

Embodiment 128. The method of Embodiment 122, wherein the product compound 4 is and cis-3-(5-acetylthiophen-2-yl)-1-(3-fluoro-2-methylphenyl)cyclopentane-1-carboxylic acid.

Embodiment 129. The method of Embodiment 122, wherein the product compound 4 is cis-3-phenylcyclopentane-1-carboxylic acid.

Embodiment 130. The method of Embodiment 122, wherein the product compound 4 is selected from the group consisting of cis-3-(4-chlorophenyl)cyclopentane-1-carboxylic acid, cis-3-(4-methoxyphenyl)cyclopentane-1-carboxylic acid, cis-3-phenylcyclopentane-1-carboxylic acid, cis-3-(4-(trifluoromethyl)phenyl)cyclopentane-1-carboxylic acid, cis-3-(3-(trifluoromethyl)phenyl)cyclopentane-1-carboxylic acid, cis-3-(2-(trifluoromethyl)phenyl)cyclopentane-1-carboxylic acid, and cis-3-(2-chloropyridin-4-yl)cyclopentane-1-carboxylic acid.

Embodiment 131. A compound having the structure of cis-1-(3-fluoro-2-methylphenyl)-3-(1-tosyl-1H-indol-5-yl)cyclopentane-1-carboxylic acid (4ao):

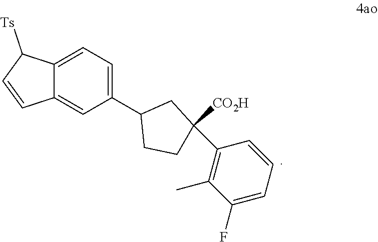 [SMILES: Cc1ccc(S(=O)(=O)C2C=Cc3cc(C4CC[C@@](C(=O)O)(c5cccc(F)c5C)C4)ccc32)cc1]

Embodiment 132. The method of Embodiment 122, wherein the product compound 4 is cis-1-(3-fluoro-2-methylphenyl)-3-(1-tosyl-1H-indol-5-yl)cyclopentane-1-carboxylic acid (4ao).

Embodiment 133. A compound having the structure of 4ap:

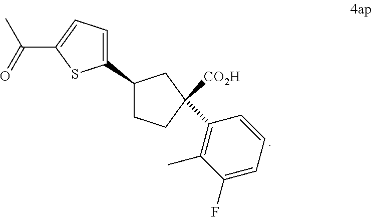 [SMILES: CC(=O)c1ccc([C@@H]2CC[C@@](C(=O)O)(c3cccc(F)c3C)C2)s1]

Embodiment 134. The method of Embodiment 122, wherein the product compound 3 is cis-3-(5-acetylthiophen-2-yl)-1-(3-fluoro-2-methylphenyl)cyclopentane-1-carboxylic acid (4ap).

Embodiment 135. The method of Embodiment 122, wherein the product compound 4 is selected from the group consisting of cis-3-(4-cyanophenyl)-1-propylcyclopentane-1-carboxylic acid, cis-3-(4-nitrophenyl)-1-propylcyclopentane-1-carboxylic acid, cis-3-(4-chlorophenyl)-1-propylcyclopentane-1-carboxylic acid, cis-3-(4-bromophenyl)-1-propylcyclopentane-1-carboxylic acid, cis-1-propyl-3-(4-(trifluoromethyl)phenyl)cyclopentane-1-carboxylic acid, cis-3-(4-fluorophenyl)-1-propylcyclopentane-1-carboxylic acid, cis-3-(4-methoxyphenyl)-1-propylcyclopentane-1-carboxylic acid, cis-3-(4-formylphenyl)-1-propylcyclopentane-1-carboxylic acid, cis-3-(4-acetylphenyl)-1-propylcyclopentane-1-carboxylic acid, cis-3-(3-(methoxycarbonyl)phenyl)-1-propylcyclopentane-1-carboxylic acid, cis-3-(3-nitrophenyl)-1-propylcyclopentane-1-carboxylic acid, cis-1-propyl-3-(3-(trifluoromethyl)phenyl)cyclopentane-1-carboxylic acid, cis-3-(3-fluorophenyl)-1-propylcyclopentane-1-carboxylic acid, cis-3-(2-fluorophenyl)-1-propylcyclopentane-1-carboxylic acid, cis-1-propyl-3-(2-(trifluoromethyl)phenyl)cyclopentane-1-carboxylic acid, cis-3-(2-cyanophenyl)-1-propylcyclopentane-1-carboxylic acid, cis-3-(3,5-bis(trifluoromethyl)phenyl)-1-propylcyclopentane-1-carboxylic acid, cis-3-(naphthalen-1-yl)-1-propylcyclopentane-1-carboxylic acid, cis-1-propyl-3-(2-(trifluoromethyl)pyridin-4-yl)cyclopentane-1-carboxylic acid, cis-3-(2-chloropyridin-4-yl)-1-propylcyclopentane-1-carboxylic acid, cis-3-(2-bromopyridin-4-yl)-1-propylcyclopentane-1-carboxylic acid, cis-3-(2-methylpyridin-4-yl)-1-propylcyclopentane-1-carboxylic acid, cis-3-(2,6-dichloropyridin-4-yl)-1-propylcyclopentane-1-carboxylic acid, cis-3-(2-chloro-6-(trifluoromethyl)pyridin-4-yl)-1-propylcyclopentane-1-carboxylic acid, cis-3-(6-chloro-5-(trifluoromethyl)pyridin-3-yl)-1-propylcyclopentane-1-carboxylic acid, cis-3-(6-chloropyridin-3-yl)-1-propylcyclopentane-1-carboxylic acid, cis-3-(6-fluoropyridin-3-yl)-1-propylcyclopentane-1-carboxylic acid, cis-3-(4-(1H-pyrrol-1-yl)phenyl)-1-propylcyclopentane-1-carboxylic acid, cis-3-(4-(1,3-dioxo-1H-benzo[de]isoquinolin-2(3H)-yl)phenyl)-1-propylcyclopentane-1-carboxylic acid, cis-1-propyl-3-(thiophen-2-yl)cyclopentane-1-carboxylic acid, cis-3-(5-acetylthiophen-2-yl)-1-propylcyclopentane-1-carboxylic acid, cis-3-(2-methylbenzo[d]thiazol-6-yl)-1-propylcyclopentane-1-carboxylic acid, cis-1-propyl-3-(quinazolin-6-yl)cyclopentane-1-carboxylic acid, cis-1-propyl-3-(quinolin-6-yl)cyclopentane-1-carboxylic acid, cis-3-(2,4-dimethoxypyrimidin-5-yl)-1-propylcyclopentane-1-carboxylic acid, and cis-1-propyl-3-(1-tosyl-1H-indol-5-yl)cyclopentane-1-carboxylic acid.

Embodiment 136. The method of Embodiment 2, wherein the method of γ-methylene C(sp³)-H arylation of cycloalkane carboxylic acids occurs according to the following reaction scheme:

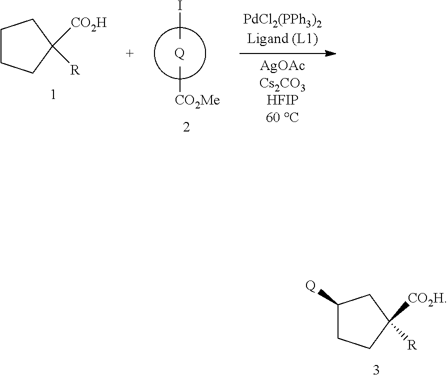

Embodiment 137. The method of Embodiment 136, wherein the product compound 3 is selected from the group consisting of cis-3-(4-(methoxycarbonyl)phenyl)-1-propylcyclopentane-1-carboxylic acid, cis-3-(4-(methoxycarbonyl)phenyl)-1-phenylcyclopentane-1-carboxylic acid, cis-1-(4-chlorophenyl)-3-(4-(methoxycarbonyl)phenyl)cyclopentane-1-carboxylic acid, cis-3-(4-(methoxycarbonyl)phenyl)-1-(p-tolyl)cyclopentane-1-carboxylic acid, cis-3-(4-(methoxycarbonyl)phenyl)-1-(4-methoxyphenyl)cyclopentane-1-carboxylic acid, cis-1-(2-fluorophenyl)-3-(4-(methoxycarbonyl)phenyl)cyclopentane-1-carboxylic acid, cis-1-(4-fluorophenyl)-3-(4-(methoxycarbonyl)phenyl)cyclopentane-1-carboxylic acid, cis-1-(3-fluoro-2-methylphenyl)-3-(4-(methoxycarbonyl)phenyl)cyclopentane-1-carboxylic acid, cis-3-(4-(methoxycarbonyl)phenyl)-1-methylcyclopentane-1-carboxylic acid, cis-1-ethyl-3-(4-(methoxycarbonyl)phenyl)cyclopentane-1-carboxylic acid, cis-1-benzyl-3-(4-(methoxycarbonyl)phenyl)cyclopentane-1-carboxylic acid, cis-1-(3-(tert-butoxy)-3-oxopropyl)-3-(4-(methoxycarbonyl)phenyl)cyclopentane-1-carboxylic acid, cis-3-(4-(methoxycarbonyl)phenyl)-1-(3-phenylpropyl)cyclopentane-1-carboxylic acid, cis-1-(cyclohexylmethyl)-3-(4-(methoxycarbonyl)phenyl)cyclopentane-1-carboxylic acid, cis-1-(4-chlorobutyl)-3-(4-(methoxycarbonyl)phenyl)cyclopentane-1-carboxylic acid, cis-3-(4-(methoxycarbonyl)phenyl)-1-(3-methoxypropyl)cyclopentane-1-carboxylic acid, and cis-3-(4-(methoxycarbonyl)phenyl)cyclopentane-1-carboxylic acid.

Embodiment 138. The method of Embodiment 2, wherein the method of γ-methylene C(sp³)-H arylation of cycloalkane carboxylic acids occurs according to the following reaction scheme:

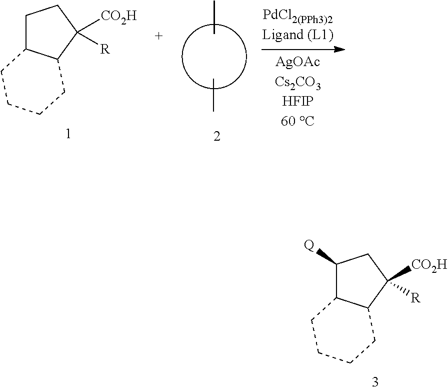

wherein [symbol] indicates single or double bonds.

Embodiment 139. The method of Embodiment 138, wherein the product compound 3 is re/-(1S,3R,3aR,7aR)-3-(4-(methoxycarbonyl)phenyl)-1-methyloctahydro-1H-indene-1-carboxylic acid or cis-3-(4-(methoxycarbonyl)phenyl)-1-propyl-2,3-dihydro-1H-indene-1-carboxylic acid.

Embodiment 140. The method of Embodiment 2, wherein the method of γ-methylene C(sp³)-H arylation of cycloalkane carboxylic acids occurs according to the following reaction scheme:

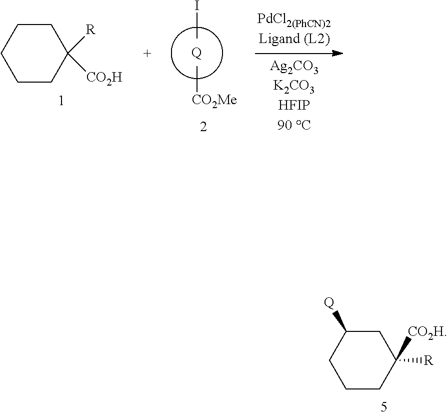

Embodiment 141. The method of Embodiment 140, wherein the product compound 5 is selected from the group consisting of cis-3-(4-(methoxycarbonyl)phenyl)-1-propylcyclohexane-1-carboxylic acid, cis-3-(4-(methoxycarbonyl)phenyl)-1-methylcyclohexane-1-carboxylic acid, cis-1-ethyl-3-(4-(methoxycarbonyl)phenyl)cyclohexane-1-carboxylic acid, cis-1-isobutyl-3-(4-(methoxycarbonyl)phenyl)cyclohexane-1-carboxylic acid, cis-1-(3-((tert-butyldimethylsilyl)oxy)propyl)-3-(4-(methoxycarbonyl)phenyl)cyclohexane-1-carboxylic acid, cis-1-(cyclohexylmethyl)-3-(4-(methoxycarbonyl)phenyl)cyclohexane-1-carboxylic acid, cis-3-(4-(methoxycarbonyl)phenyl)-1-(3-phenoxypropyl)cyclohexane-1-carboxylic acid, cis-1-benzyl-3-(4-(methoxycarbonyl)phenyl)cyclohexane-1-carboxylic acid, cis-3-(4-(methoxycarbonyl)phenyl)-1-phenylcyclohexane-1-carboxylic acid, cis-3-(4-(methoxycarbonyl)phenyl)-1-(p-tolyl)cyclohexane-1-carboxylic acid, cis-3-(4-(methoxycarbonyl)phenyl)-1-(4-methoxyphenyl)cyclohexane-1-carboxylic acid, cis-5-(4-(methoxycarbonyl)phenyl)-1,3,3-trimethylcyclohexane-1-carboxylic acid, cis-2-(4-(methoxycarbonyl)phenyl)-4-propyltetrahydro-2H-pyran-4-carboxylic acid, and (2R,4R,4aS,6aR,9S,11aR,11bS)-2-(4-(methoxycarbonyl)phenyl)-4,9,11b-trimethyl-8-oxotetradecahydro-6a,9-methanocyclohepta[a]naphthalene-4-carboxylic acid.

Embodiment 142. The method of Embodiment 2, wherein the method of γ-methylene C(sp³)-H arylation of cycloalkane carboxylic acids occurs according to the following reaction scheme:

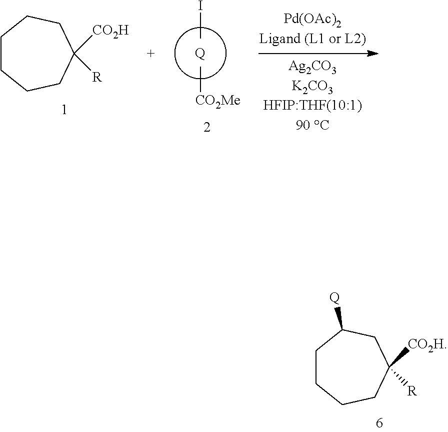

Embodiment 143. The method of Embodiment 142, wherein the product compound 6 is selected from the group consisting of cis-3-(4-(methoxycarbonyl)phenyl)-1-propylcycloheptane-1-carboxylic acid, cis-3-(4-(methoxycarbonyl)phenyl)-1-methylcycloheptane-1-carboxylic acid, cis-1-ethyl-3-(4-(methoxycarbonyl)phenyl)cycloheptane-1-carboxylic acid, cis-3-(4-(methoxycarbonyl)phenyl)-1-(3-methoxypropyl)cycloheptane-1-carboxylic acid, cis-1-(3-((tert-butyldimethylsilyl)oxy)propyl)-3-(4-(methoxycarbonyl)phenyl)cycloheptane-1-carboxylic acid, cis-1-(5-chloropentyl)-3-(4-(methoxycarbonyl)phenyl)cycloheptane-1-carboxylic acid, cis-1-(cyclohexylmethyl)-3-(4-(methoxycarbonyl)phenyl)cycloheptane-1-carboxylic acid, and cis-3-(4-(methoxycarbonyl)phenyl)-1-(3-phenylpropyl)cycloheptane-1-carboxylic acid.

Embodiment 144. The method of Embodiment 2, wherein the method of γ-methylene C(sp³)-H arylation of cycloalkane carboxylic acids occurs according to the following reaction scheme:

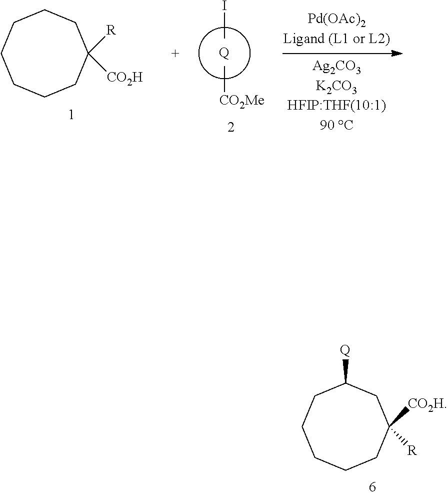

Embodiment 145. The method of Embodiment 144, wherein the product compound 6 is cis-3-(4-(methoxycarbonyl)phenyl)-1-propylcyclooctane-1-carboxylic acid or cis-3-(4-(methoxycarbonyl)phenyl)-1-methylcyclooctane-1-carboxylic acid.

Embodiment 146. A compound having the structure of L1:

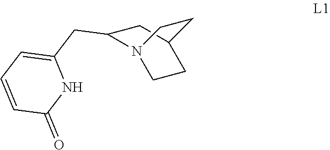 [SMILES: O=c1cccc(CC2CC3CCN2CC3)[nH]1]

including any enantiomer, scalemic or racemic mixture, or pharmaceutically acceptable salt thereof.

Embodiment 147. A method of preparing the compound of Embodiment 146, according to the following scheme:

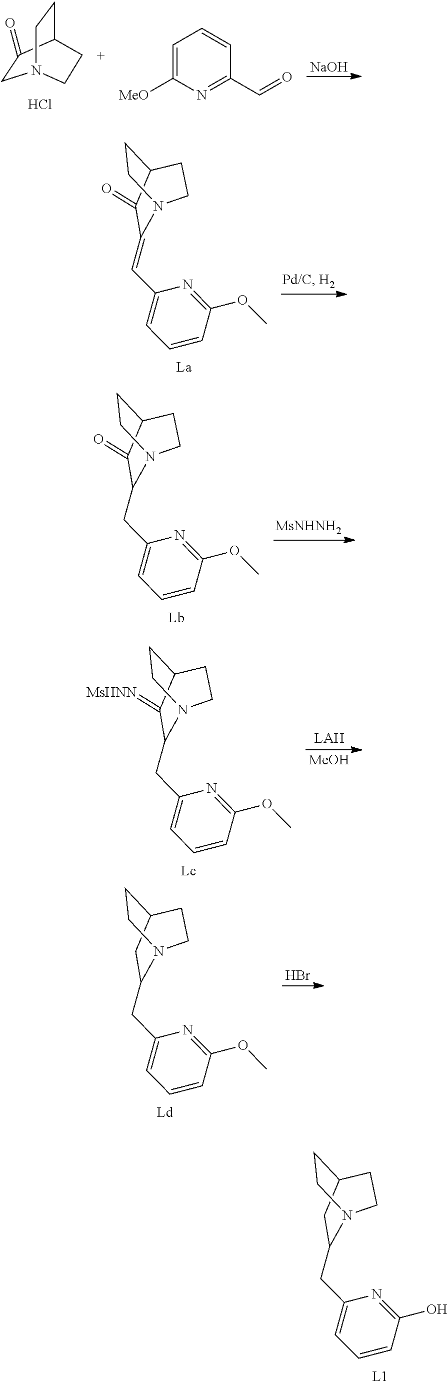

Embodiment 148. A compound having the structure of L2:

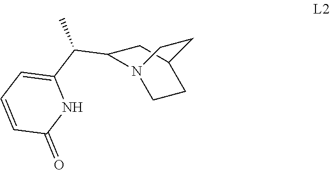 [SMILES: C[C@@H](c1cccc(=O)[nH]1)C1CC2CCN1CC2]

including any enantiomer, scalemic or racemic mixture, or pharmaceutically acceptable salt thereof.

Embodiment 149. A method of preparing the compound of Embodiment 148, according to the following scheme:

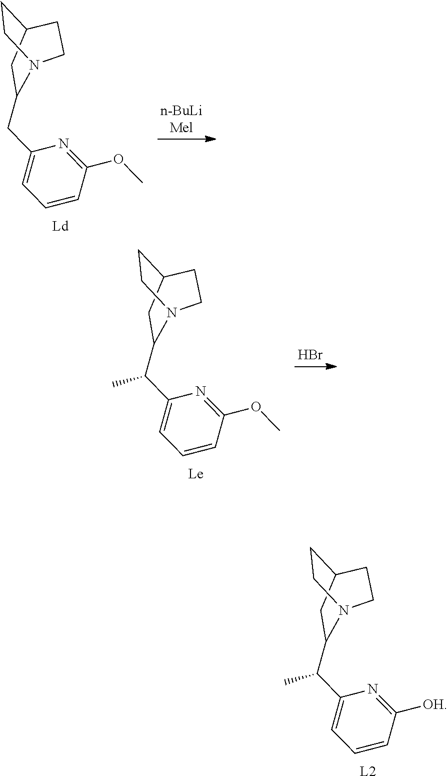

Embodiment 150. Any method of γ-methylene C(sp³)-H arylation of cycloalkane carboxylic acids, ligands, or preparation thereof as described herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “ [symbol] ” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH- [symbol] -C(—OH)═CH—), amide/imidic acid (—C(═O)—NH- [symbol] -C(—OH)═N—) and amidine (—C(═NR)—NH- [symbol] -C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino,dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}—, —C(═NR^{bb})OR^{aa}, —OC(═NR^{bb})R^{aa}, —OC(═NR^{bb})OR—, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R—, —SO₂N(R^{bb})₂, —SO₂R—, —S(═O)R^{aa}, —OS(═O)R^{aa}, —B(OR^{cc})₂, C₁-10 alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁-10 alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁-10 alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{oo} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl, and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### DETAILED DESCRIPTION OF THE FIGURES

FIG. 1. Transannular C—H arylation via activation of methylene C—H bonds a, Molecular editing logic for the synthesis of complex carbocycles b, Transannular C—H activation reactions. c, γ-Arylated carbocycle containing bioactive molecules. d, (this work) Transannular C—H functionalization of small to medium sized cycloalkane carboxylic acids.

FIG. 2. Substrate scope of cyclopentane carboxylic acids. Bolded bonds indicate relative stereochemistry. Reaction conditions: Substrate (0.1 mmol), PdCl₂(PPh₃)₂ 10 mol %, ArI 2a (2.0 equiv.), Ligand L1 (10 mol %), AgOAc (1.5 equiv.), Cs₂CO₃ (1.5 equiv.), HFIP (0.6 mL), 60° C., 24 h. Isolated yields are reported.

FIG. 3. Transannular γ-C—H arylation: Bolded bonds indicate relative stereochemistry. a, Substrate scope of aryl iodide. b, Substrate scope of heteroaryl iodide. c, i) Histone deacetylase (HDAC) inhibitor precursors. ii) AKR1C1 & AKR1C3 inhibitor and analogues. Reaction conditions: Substrate (0.1 mmol), PdCl₂(PPh₃)₂ (10 mol %), (hetero)ArI (2.0 equiv.), Ligand L1 (10 mol %), AgOAc (1.5 equiv.), Cs₂CO₃ (1.5 equiv.), HFIP (0.6 mL), 60° C., 24 h. Isolated yields are reported.

FIG. 4. Transannular C—H arylation of cycloalkane carboxylic acids. Bolded bonds indicate relative stereochemistry (A) 6-membered ring transannular C—H arylation. Reaction conditions: Substrate (0.1 mmol), PdCl₂(PhCN)₂ 10 mol %, Ligand L2 (20 mol %), ArI (2.0 equiv.), AgCO₃ (1.5 equiv.), K₂CO₃ (1.5 equiv.), HFIP (1.0 mL), 90° C., 24 h. Isolated yields are reported. *0.1 mL CH₃CN was added. (B) 7- and 8-membered ring transannular C—H arylation. Reaction conditions: Substrate (0.1 mmol), Pd(OAc)₂ (10 mol %), Ligand L2 (15 mol %), ArI (2.0 equiv.), AgCO₃ (1.5 equiv.), K₂CO₃ (3.0 equiv.), HIWP (1.0 mL) and THE (0.1 mL), 90° C., 24 h. t Substrate (0.1 mmol), Pd(OAc)₂ (10 mol %), Ligand L1 (15 mol %), ArI (2.0 equiv.), AgCO₃ (1.5 equiv.), Na₃PO₄ (1.5 equiv.), HFIP (0.6 mL), 120° C., 48 h.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Information:

Pd(OAc)₂ was purchased from Strem. Solvents were obtained from Sigma-Aldrich, Alfa-Aesar, and Acros, and used directly without further purification. Other reagents were purchased at the highest commercial quality and used without further purification, unless otherwise stated. Analytical thin layer chromatography was performed on 0.25 mm silica gel 60-F254 or Merck pre-coated aluminium-backed silica gel F254 plates. ¹H NMR spectra were recorded on Bruker AMX-400, Bruker AV NEO-500, or Bruker DRX-600 instruments. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 and were fully decoupled by broad band proton decoupling. Chemical shifts were referenced to the appropriate residual solvent peaks. 19F NMR spectra were recorded on a Bruker AV-NEO 399 instrument. Column chromatography was performed using E Merck silica (60, particle size 0.043-0.063 mm), and pTLC was performed on Merck silica plates (60F-254). High-resolution mass spectra (HRMS) were recorded on an Agilent Mass spectrometer using ESI-TOF (electrospray ionization-time of flight).

### Experimental Section for Transannular C—H Arylation

### Optimized Procedure for the Preparation of Ligands L1-L2

The synthesis of L1-L2 is detailed below. Pyridone L4 is commercially available and was used as received.

### Procedure A: Synthesis of 6-(quinuclidin-2-ylmethyl)pyridin-2-ol (L1)

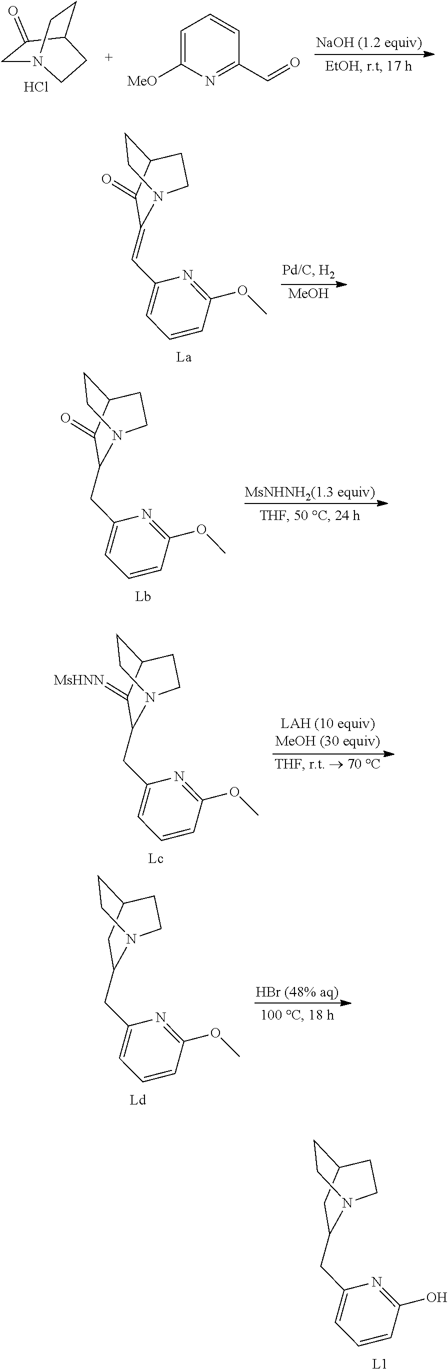

### Synthesis of La

To a solution of quinuclidin-3-one hydrochloride (50 mmol, 1.0 equiv., 8.08 g) in EtOH (50 mL) at 23° C. was added NaOH (50% in H₂O, 60 mmol, 1.2 equiv., 4.8 g). The resulting solution was stirred for 5 min before 6-methoxypicolinaldehyde (50 mmol, 1.0 equiv., 6.86 g) in 15 mL EtOH was added. The reaction mixture was allowed to stir overnight. Upon completion, the yellow solid was filtered and washed with 30 ml H₂O. The solid was dried under high vacuum and used directly in the next step. (70%~90% yield).

### Synthesis of Lb

To a solution of La (37.8 mmol, 1.0 equiv, 9.22 g) in anhydrous MeOH (100 mL) at 23° C. was added Pd/C (10 wt. %, 922 mg.). Then, a balloon of H₂ was applied. The resulting suspension was allowed to stir for 5 h. Upon completion (monitored by TLC), the reaction mixture was filtered through celite to remove the Pd/C. The filtrate was concentrated under vacuum and purified through flash column chromatography (eluent: ethyl acetate) to afford Lb (5.89 g, 63% yield).

Note: long reaction time will result in by-product formation.

### Synthesis of Lc

A solution of Lb (17.3 mmol, 1.0 equiv, 4.25 g) and MsNHNH₂ (25.9 mmol, 1.5 equiv, 2.86 g) in THF (30 mL) was heated to 50° C. and stirred for 24 hours. Upon completion (determined by TLC monitoring), the reaction mixture was cooled to room temperature and passed through filtration paper. The solid was washed with ethyl acetate (20 mL). Then the solid was dried in high vacuum to get Lc (5.5 g, 94% yield)

### Synthesis of Ld

To a solution of LAH (1 M in THF) (30 mmol, 6.0 equiv., 30 mL) at 0° C. under N₂ was added MeOH (90 mmol, 18 equiv., 3.64 mL) dropwise over 20 min and stirred for additional 10 min. Then, the resulting solution was added dropwise over 30 min to a solution of Lc (5 mmol, 1.0 equiv., 1.69 g) in THE (160 mL) at 0° C. under N₂. After the addition, the reaction mixture was heated to 70° C. and stirred overnight (~18 h). Upon completion (monitored by TLC), the mixture was cooled to room temperature and treated with saturated NaHCO₃ aqueous solution (20 mL) dropwise. The generated white solid was filtered and washed with ethyl acetate (50 mL). The filtrate was concentrated under vacuum and purified with flash column chromatography (eluent: MeOH/ethyl acetate/Et₃N=50:50:1) to afford Ld (646 mg, 55% yield).

Note: There is some solubility of Ld in H₂O. Using too much of the saturated NaHCO₃ solution will reduce the yield.

### Synthesis of L1

A solution of Ld (1.48 mmol, 343 mg) in 2 mL HBr (48% aq) was heated to 100° C. in a sealed vial and stirred overnight. Upon completion (monitored by TLC), the reaction mixture was cooled to room temperature and basified with saturated NaHCO₃. The resulting mixture was concentrated under vacuum to remove all solvent and extracted with CHCl₃ (3×50 mL). The extracts were combined, dried over anhydrous Na₂SO₄ and purified with flash column chromatography (eluent: MeOH/ethyl acetate/Et₃N=50:50:1) to afford L1 (171 mg, 53% yield).

Note: After the reaction mixture was basified, all aqueous solution should be removed due to solubility of the product in it. Other demethylation conditions might give higher yield.

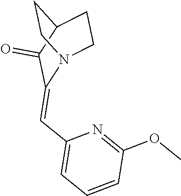 [SMILES: COc1cccc(/C=C2/C(=O)C3CCN2CC3)n1]

### (Z)-2-((6-methoxypyridin-2-yl)methylene)quinuclidin-3-one (La)

Yellow solid, H NMR (600 MHz, CDCl₃) δ 8.17 (dd, J=7.4, 0.8 Hz, 1H), 7.57 (ddd, J=8.0, 7.4, 0.5 Hz, 1H), 7.08 (s, 1H), 6.67 (dd, J=8.2, 0.8 Hz, 1H), 3.92 (s, 3H), 3.21-3.12 (m, 2H), 3.06-2.96 (m, 2H), 2.65 (p, J=3.0 Hz, 1H), 2.04 (td, J=7.9, 3.1 Hz, 4H). ¹³C NMR (151 MHz, CDCl₃) δ 206.0, 163.6, 150.6, 146.7, 138.6, 126.0, 120.5, 111.2, 53.4, 47.5, 40.4, 25.9. HRMS (ESI-TOF) m/z Calcd for C₁₄H₁₇N₂O₂⁺ [M+H]+ 245.1290, found 245.1289.

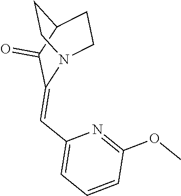 [SMILES: COc1cccc(/C=C2/C(=O)C3CCN2CC3)n1]

### 2-((6-methoxypyridin-2-yl)methyl)quinuclidin-3-one (Lb)

White solid, ¹H NMR (600 MHz, CDCl₃) δ 7.48-7.45 (m, 1H), 6.74 (d, J=7.2 Hz, 1H), 6.55 (d, J=8.4 Hz, 1H), 3.92 (dd, J=10.7, 4.1 Hz, 1H), 3.90-3.89 (m, 3H), 3.27 (dd, J=15.0, 3.6 Hz, 1H), 3.26-3.17 (m, 1H), 3.14-3.06 (m, 1H), 2.98-2.89 (m, 1H), 2.89-2.80 (m, 2H), 2.50-2.44 (m, 1H), 2.07-1.94 (m, 4H). ¹³C NMR (151 MHz, CDCl₃) δ 221.7, 163.8, 156.4, 139.0, 115.9, 108.4, 69.8, 53.3, 49.0, 41.3, 40.3, 36.0, 27.0, 25.2. HRMS (ESI-TOF) m/z Calcd for C₁₄H₁₉N₂O₂⁺ [M+H]⁺ 247.1447, found 247.1445.

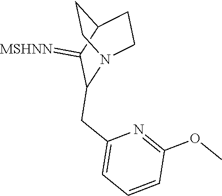

### (Z)—N′-(2-((6-methoxypyridin-2-yl)methyl)quinuclidin-3-ylidene)methanesulfonohydrazide (Lc)

White solid, NMR data was reported for the major isomer of the mixture. ¹H NMR (600 MHz, CDCl₃) δ 9.84 (s, 1H), 7.53 (dd, J=8.3, 7.1 Hz, 1H), 6.79 (d, J=7.2 Hz, 1H), 6.67 (d, J=8.3 Hz, 1H), 4.05 (dd, J=6.0, 3.3 Hz, 1H), 4.03 (s, 3H), 3.37-3.27 (m, 1H), 3.23-3.17 (m, 1H), 3.09 (s, 3H), 3.06-3.02 (m, 1H), 2.99-2.91 (m, 1H), 2.92-2.83 (m, 1H), 2.78-2.74 (m, 1H), 2.73-2.71 (m, 1H), 1.94-1.84 (m, 4H). ¹³C NMR (151 MHz, CDCl₃) δ 164.71, 164.65, 156.41, 139.78, 116.11, 109.76, 61.97, 54.19, 49.82, 41.72, 38.86, 35.82, 32.84, 27.85, 25.26. HRMS (ESI-TOF) m/z Calcd for C₁₅H₂₃N₄O₃S⁺[M+H]⁺ 339.1491, found 339.1487.

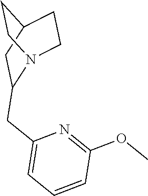 [SMILES: COc1cccc(CC2CC3CCN2CC3)n1]

### 2-((6-methoxypyridin-2-yl)methyl)quinuclidine (Ld)

Sticky liquid. ¹H NMR (600 MHz, CDCl₃) δ 7.45 (dd, J=8.2, 7.2 Hz, 1H), 6.73 (dd, J=7.2, 0.7 Hz, 1H), 6.53 (dd, J=8.2, 0.8 Hz, 1H), 3.91 (s, 3H), 3.38-3.28 (m, 1H), 3.16-3.06 (m, 1H), 2.99-2.89 (m, 3H), 2.82-2.71 (m, 2H), 1.79-1.72 (m, 1H), 1.69-1.61 (m, 1H), 1.56-1.43 (m, 4H), 1.29-1.18 (m, 1H). ¹³C NMR (151 MHz, CDCl₃) δ 163.7, 157.9, 138.8, 116.1, 107.6, 55.9, 53.3, 50.1, 43.8, 41.9, 33.7, 27.1, 25.8, 22.1. HRMS (ESI-TOF) m/z Calcd for C₁₄H₂₁N₂O⁺ [M+H]⁺ 233.1654, found 233.1662.

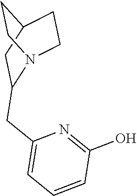 [SMILES: Oc1cccc(CC2CC3CCN2CC3)n1]

### 6-(quinuclidin-2-ylmethyl)pyridin-2-ol (Li)

White solid. ¹H NMR (600 MHz, CDCl₃) δ 7.27-7.23 (m, 1H), 6.38 (dt, J=9.1, 1.0 Hz, 1H), 5.94 (d, J=6.8 Hz, 1H), 3.21 (q, J=8.9 Hz, 1H), 3.15-2.99 (m, 3H), 2.95-2.85 (m, 2H), 2.39 (dd, J=16.1, 3.3 Hz, 1H), 1.94-1.82 (m, 2H), 1.65-1.52 (m, 4H), 1.20-1.12 (m, 1H). ¹³C NMR (151 MHz, CDCl₃) δ 164.4, 148.4, 140.8, 117.9, 104.9, 55.7, 49.1, 40.3, 33.9, 33.6, 26.4, 25.1, 21.6. HRMS (ESI-TOF) m/z Calcd for C₁₃H₁₈N₂O⁺, [M+H]⁺ 219.1497, found 219.1497.

### Procedure B: Synthesis of 6-(1-(quinuclidin-2-yl)ethyl)pyridin-2-ol (L2)

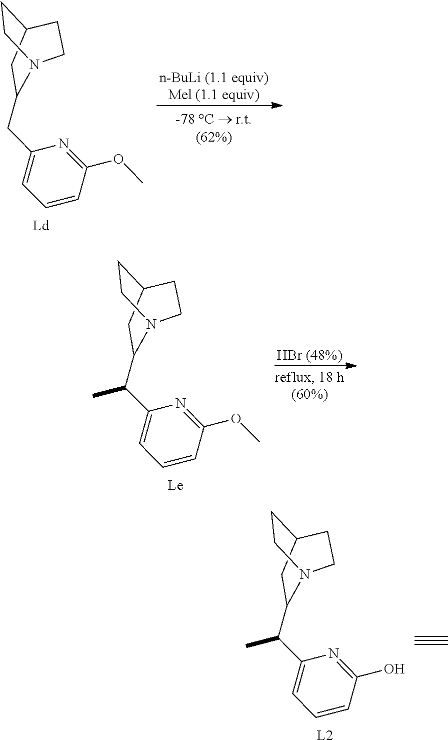

To a solution of Ld (2.72 mmol, 632 mg) in 8 mL THE at −78° C. under nitrogen atmosphere was added n-BuLi (2.5 M in hexanes) (3.00 mmol, 1.1 equiv, 1.20 mL) dropwise over 20 min. The reaction mixture was allowed to stir for 1 h before the addition of Mel (3.00 mmol, 1.1 equiv., 425.8 mg). The resulting mixture was warmed to room temperature gradually and stirred overnight. After completion, the mixture was concentrated and purified with flash column chromatography (eluent: MeOH/ethyl acetate=0→50%) to afford Le (421 mg, 62% yield). A solution of Le (1.36 mmol, 334 mg) in 2 mL HBr (48% aq) was heated to 100° C. in a sealed vial and stirred overnight. Upon completion (monitored by TLC), the reaction mixture was cooled to room temperature and basified with saturated NaHCO₃. The resulting mixture was concentrated under vacuum to remove all solvent and extracted with CHCl₃ (3×50 mL). The extracts were combined, dried over anhydrous Na₂SO₄ and purified with flash column chromatography (eluent: MeOH/ethyl acetate/Et₃N=50:50:1) to afford L2 (189 mg, 60% yield).

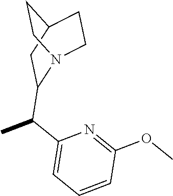 [SMILES: COc1cccc([C@@H](C)C2CC3CCN2CC3)n1]

### 2-(1-(6-methoxypyridin-2-yl)ethyl)quinuclidine (Le)

Colorless liquid. ¹H NMR (600 MHz, CDCl₃) δ 7.59 (dd, J=8.3, 7.2 Hz, 1H), 6.85 (d, J=7.4 Hz, 1H), 6.72 (dd, J=8.4, 0.8 Hz, 1H), 3.96 (s, 3H), 3.97-3.90 (m, 1H), 3.80-3.72 (m, 1H), 3.72-3.65 (m, 1H), 3.39-3.26 (m, 2H), 3.22-3.17 (m, 1H), 2.32 (s, 1H), 2.30-2.22 (m, 1H), 2.00-1.86 (m, 4H), 1.98-1.87 (m, 1H), 1.32 (d, J=6.9 Hz, 3H). ¹³C NMR (151 MHz, CDCl₃) δ 164.9, 156.7, 140.6, 115.9, 111.0, 61.6, 54.1, 49.9, 42.6, 42.4, 30.3, 23.3, 22.5, 21.3, 18.2. HRMS (ESI-TOF) m/z Calcd for C₁₅H₂₃N₂O, [M+H]⁺ 247.1810, found 247.1806.

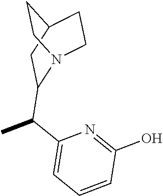 [SMILES: C[C@H](c1cccc(O)n1)C1CC2CCN1CC2]

### 6-(1-(quinuclidin-2-yl)ethyl)pyridin-2-ol (L2)

White solid. ¹H NMR (500 MHz, CDCl₃) δ 13.34 (s, 1H), 7.29 (dd, J=9.1, 7.0 Hz, 1H), 6.37 (d, J=9.1 Hz, 1H), 6.02 (d, J=7.0 Hz, 1H), 3.06-2.91 (m, 3H), 2.90-2.77 (m, 2H), 2.72-2.62 (m, 1H), 1.88-1.82 (m, 2H), 1.60-1.47 (m, 4H), 1.33-1.25 (m, 1H), 1.21 (d, J=6.8 Hz, 3H). ¹³C NMR (151 MHz, CDCl₃) δ 164.4, 153.5, 140.7, 118.1, 102.5, 60.5, 48.7, 40.6, 34.6, 32.8, 26.6, 25.2, 21.9, 13.4. HRMS (ESI-TOF) m/z Calcd for C₁₄H₂₁N₂O⁺, [M+H]⁺ 233.1654, found 233.1664. The structure and relative stereochemistry of L2 were confirmed by X-ray crystallography.

### 2.3.1. Condition Screening for Cyclopentane Carboxylic Acids

TABLE S1
Ligand Effects^{a,b}

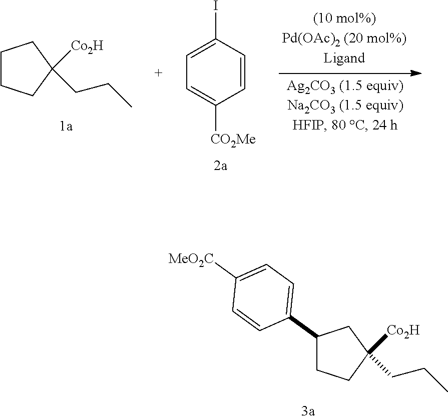

| | | | |
|---|---|---|---|
| 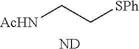 | 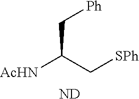 | 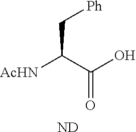 [SMILES: CC(=O)N[C@@H](Cc1ccccc1)C(=O)O ; "ND"] | 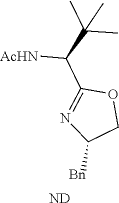 [SMILES: CC(=O)N[C@H](C1=N[C@@H](Cc2ccccc2)CO1)C(C)(C)C ; "ND"] |
| 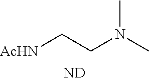 | 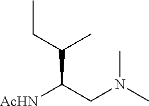 ND | 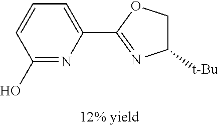 [SMILES: CC(C)(C)[C@H]1COC(c2cccc(O)n2)=N1] | 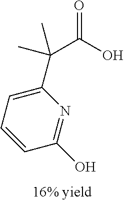 |
| 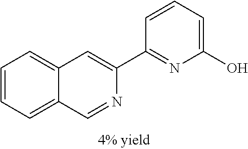 [SMILES: Oc1cccc(-c2cc3ccccc3cn2)n1] | 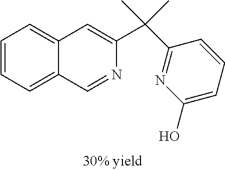 [SMILES: CC(C)(c1cc2ccccc2cn1)c1cccc(O)n1] | 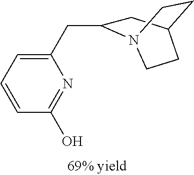 [SMILES: Oc1cccc(CC2CC3CCN2CC3)n1] | 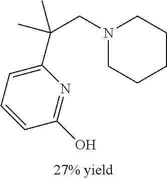 [SMILES: CC(C)(CN1CCCCC1)c1cccc(O)n1] |

^{a}Conditions: 1a (0.1 mmol), methyl 4-iodobenzoate 2a (0.2 mmol), Pd(OAc)₂ (10 mol %), ligand (20 mol %), Ag₂CO₃ (1.5 equiv), Na₂CO₃ (1.5 equiv), HFIP (0.6 mL), 80 °C, 24 h.
^{b}yields determined by ¹H NMR using dibromomethane as internal standard

TABLE S2
Solvent Effects^{a,b}

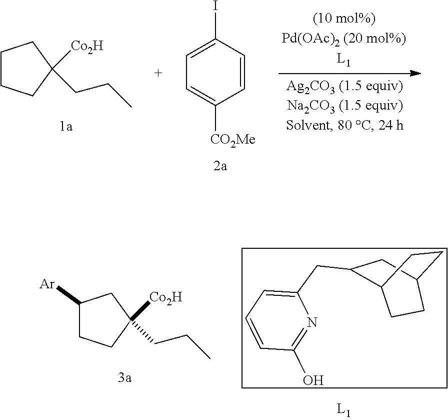

| entry | solvent | yield (%) |
|---|---|---|
| 1 | HFIP | 69 |
| 2 | CHCl₃ | 3 |
| 3 | toluene | 14 |
| 4 | DMF | 9 |
| 5 | THF | 6 |
| 6 | dioxane | 11 |
| 7 | CH₃CN | 8 |
| 8 | t-amylol | 9 |

^{a}Conditions: 1a (0.1 mmol), methyl 4-iodobenzoate 2a (0.2 mmol), Pd(OAc)₂ (10 mol %), L1 (20 mol %), Ag₂CO₃ (1.5 equiv), Na₂CO₃ (1.5 equiv), solvent (0.6 mL), 80 °C, 24 h.
^{b}yields determined by ¹H NMR using dibromomethane as internal standard

TABLE S3
Pd Source Screening^{a,b}

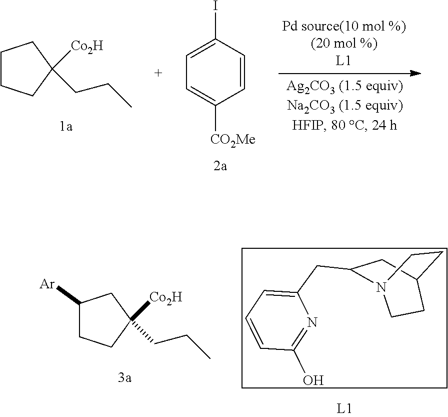

| entry | Pd source | yield (%) |
|---|---|---|
| 1 | Pd(OAc)₂ | 69 |
| 2 | PdCl₂allyl₂ | 67 |
| 3 | Pd(TFA)₂ | 71 |
| 4 | PdCl_{₂}_{(PhCN)}_{₂} | 78 |
| 5 | PdCl_{2(ethylenediamine)} | nd |
| 6 | PdCl_{2(PPh3)2} | 79 |
| 7 | Pd(dba)3 | 73 |

^{a}Conditions: 1a (0.1 mmol), methyl 4-iodobenzoate 2a (0.2 mmol), Pd source (10 mol %), L1 (20 mol %), Ag₂CO₃ (1.5 equiv), Na₂CO₃ (1.5 equiv), HFIP (0.6 mL), 80 ° C, 24 h.
^{b}yields determined by ¹H NMR using dibromomethane as internal standard

TABLE S4
Oxidant Source Screening^{a,b}

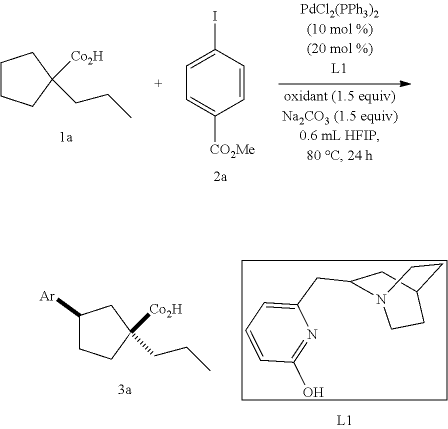

| entry | oxidant | yield (%) |
|---|---|---|
| 1 | Ag₂CO₃ | 79 |
| 2 | Ag₂O | 76 |
| 3 | AgNO₃ | 60 |
| 4 | AgPO₄ | 65 |
| 5 | AgTFA | 74 |
| 6 | AgOAc | 81 |
| 7 | Na₂CO₃•1.5H₂O₂ | 12 |
| 8 | PIDA | nd |

^{a}Conditions: 1a (0.1 mmol), methyl 4-iodobenzoate 2a (0.2 mmol), PdCl₂(PPh₃)₂ (10 mol %), L1 (20 mol %), oxidant (1.5 equiv), Na₂CO₃ (1.5 equiv), HFIP (0.6 mL), 80 °C, 24 h.
^{b}yields determined by ¹H NMR using dibromomethane as internal standard

TABLE S5
Screenings of Base^{a,b}

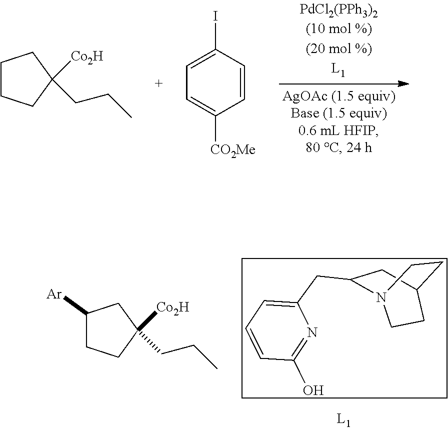

| entry | Base | yield (%) |
|---|---|---|
| 1 | Na₂CO₃ | 81 |
| 2 | Li₂CO₃ | 65 |
| 3 | K₂CO₃ | 79 |
| 4 | Cs₂CO₃ | 82 |
| 5 | NaHCO₃ | 71 |
| 6 | K₂HPO₄ | 56 |
| 7 | KH₂PO₄ | 67 |
| 8 | Na₃PO₄ | 61 |
| 9 | Na₂HPO₄•7H₂O | 77 |
| 10 | t-BuOK | 75 |
| 11 | CH₃CO₂Na | 65 |
| 12 | Na₂SO₃ | 66 |

^{a}Conditions: 1a (0.1 mmol), methyl 4-iodobenzoate 2a (0.2 mmol), PdCl₂(PPh₃)₂ (10 mol %), L1 (20 mol %), AgOAc (1.5 equiv), Base (1.5 equiv), HFIP (0.6 mL), 80 °C, 24 h.
^{b}yields determined by ¹H NMR using dibromomethane as internal standard

TABLE S6
Temperature and Concentration Screening^{a,b}

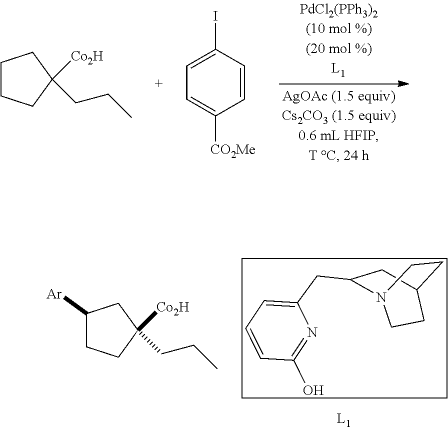

| entry | T °C | yield (%) |
|---|---|---|
| 1 | 120 | 67 |
| 2 | 100 | 76 |
| 3 | 80 | 82 |
| 4 | 60 | 90 |
| 5 | 40 | 31 |
| 6 | 20 | nd |
| 7 | 0.4 | 73 |
| 8 | 0.8 | 81 |
| 9 | 1.0 | 79 |

^{a}Conditions: 1a (0.1 mmol), methyl 4-iodobenzoate 2a (0.2 mmol), PdCl₂(PPh₃)₂ (10 mol %), L1 (20 mol %), AgOAc (1.5 equiv), Cs₂CO₃ (1.5 equiv), HFIP (× mL), T ° C, 24 h.
^{b}yields determined by ¹H NMR using dibromomethane as internal standard

TABLE S7
Equivalents of Reagents Screening^{a}

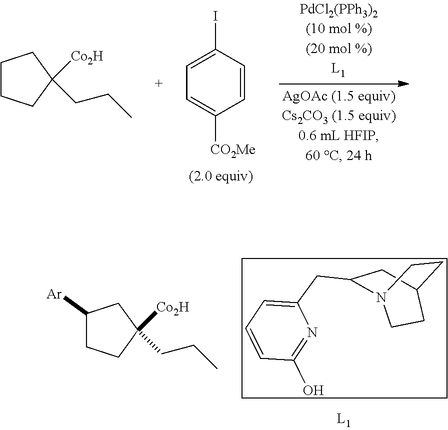

| entry | virants | yield (%)^{a} |
|---|---|---|
| 1 | no change | 90 |
| 2 | 1.5 equiv Arl | 79 |
| 3 | 1.0 equiv AgOAc | 73 |
| 4 | 0.5 equiv AgOAc | 44 |
| 5 | 1.0 equiv Cs₂CO₃ | 86 |
| 6 | 0.5 equiv Cs₂CO₃ | 70 |
| 7 | 5 mol % PdCl_{₂}_{(PPh}_{₃}₎_{₂}_{,} 10 mol % Ligand | 77 |
| 8 | 10 mol % PdCl_{₂}_{(PPh3)}_{₂}_{,} 10 mol % Ligand | 99 (87)^{b} |
| 9 | 10 mol % PdCl_{2(PPh}_{₃}₎_{₂}_{,} 15 mol % Ligand | 86 |

^{a}Conditions: substrate (0.1 mmol), methyl 4-iodobenzoate 2a (0.2 mmol), PdCl₂(PPh₃)₂ (10 mol %), L1 (20 mol %), AgOAc (1.5 equiv), Cs₂CO₃ (1.5 equiv), HFIP (0.6 mL), 60 °C, 24 h, all with variations as noted above.
^{b}Isolated yield

Optimization for Transannular γ-C—H Arylation of α-propyl Cyclohexane Carboxylic Acid
Reaction with Optimal Conditions for Cyclopentane Carboxylic Acid

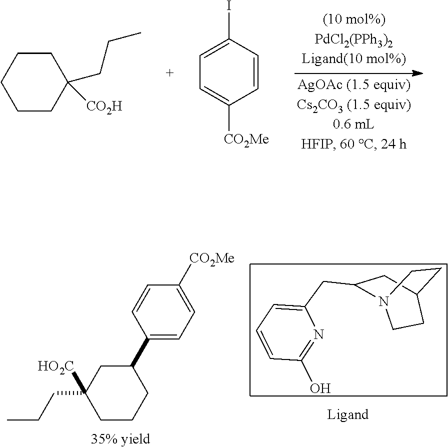

TABLE S8
Screenings of Ligands^{a,b}

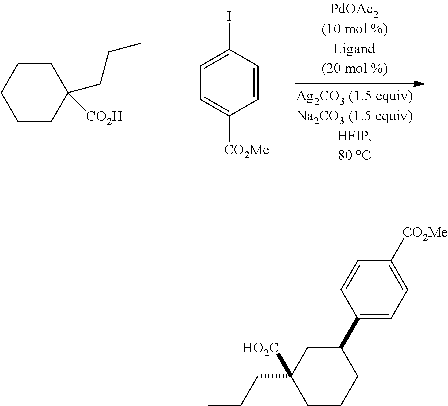

| | | | |
|---|---|---|---|
| 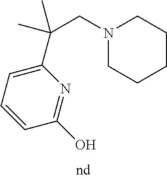 [SMILES: CC(C)(CN1CCCCC1)c1cccc(O)n1] | 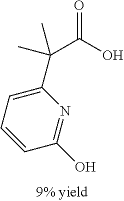 | 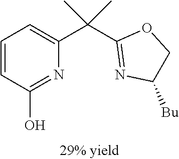 | 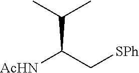 nd |
| 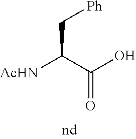 [SMILES: CC(=O)N[C@@H](Cc1ccccc1)C(=O)O] | 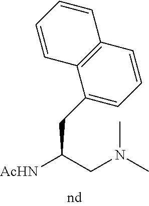 [SMILES: CC(=O)N[C@@H](Cc1cccc2ccccc12)CN(C)C] | 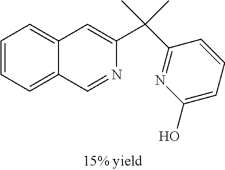 [SMILES: CC(C)(c1cc2ccccc2cn1)c1cccc(O)n1] | 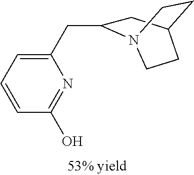 [SMILES: Oc1cccc(CC2CC3CCN2CC3)n1] |
| 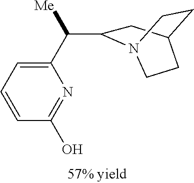 [SMILES: C[C@H](c1cccc(O)n1)C1CC2CCN1CC2] | | | |

^{a}Conditions: substrate (0.1 mmol), methyl 4-iodobenzoate 2a (0.2 mmol), Pd(OAc₂ (10 mol %), ligand (20 mol %), Ag₂CO₃ (1.5 equiv), Na₂CO₃ (1.5 equiv), HFIP (0.6 mL), 80 °C, 24 h.
^{b}yields determined by ¹H NMR using dibromomethaneas internal standard

TABLE S9
Pd source screening^{a,b}

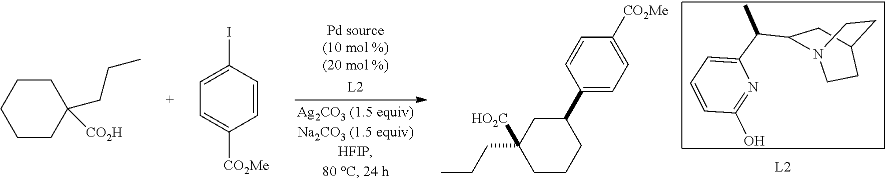

| entry | Pd source | yield (%) |
|---|---|---|
| 1 | Pd(OAc)₂ | 57 |
| 2 | PdCl₂allyl₂ | 52 |
| 3 | Pd(TFA)₂ | 53 |
| 4 | PdCl_{₂}_{(PhCN)}_{₂} | 61 |
| 5 | PdCl₂ | nd |
| 6 | PdCl_{₂}_{(PPh}_{₃}₎_{₂} | 52 |
| 7 | Pd(dba)3 | 58 |
| 8 | PdCl_{2(dppf)} | 42 |

^{a}Conditions: substrate (0.1 mmol), methyl 4-iodobenzoate 2a (0.2 mmol), Pd source (10 mol %), L2 (20 mol %), Ag₂CO₃ (1.5 equiv), Na₂CO₃ (1.5 equiv), HFIP (0.6 mL), 80 ° C, 24 h
^{b}yields determined by ¹H NMR using dibromomethane as internal standard

TABLE S10
Base screening^{a,b}

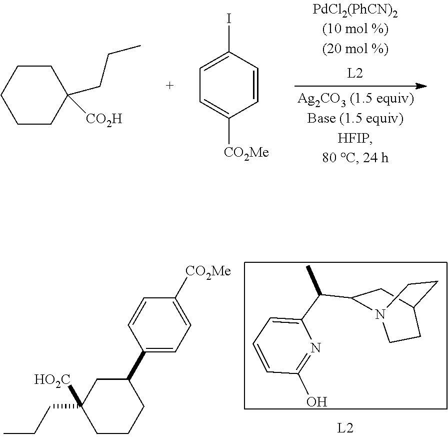

| entry | Base | yield (%) |
|---|---|---|
| 1 | Na₂CO₃ | 63 |
| 2 | Li₂CO₃ | 62 |
| 3 | K₂CO₃ | 65 |
| 4 | Cs₂CO₃ | 61 |
| 5 | NaHCO₃ | 57 |
| 6 | K₂HPO₄ | 56 |
| 7 | KH₂PO₄ | 56 |
| 8 | Na₃PO₄ | 62 |
| 9 | Li₃PO₄ | 27 |
| 10 | KF | 38 |
| 11 | t-BuOK | 64 |
| 12 | t-AmONa | 48 |
| 13 | HCO₂Na | 45 |
| 14 | CH₃CO₂Na | 37 |
| 15 | CH₃CO₂K | 24 |
| 16 | NaTFA | 15 |

^{a}Conditions: substrate (0.1 mmol), methyl 4-iodobenzoate 2a (0.2 mmol), PdCl₂(PhCN)₂ (10 mol %), L2 (20 mol %), Ag₂CO₃ (1.5 equiv), Base (1.5 equiv), HFIP (0.6 mL), 80 °C, 24 h.
^{b}yields determined by ¹H NMR using dibromomethane as internal standard

TABLE S11
Oxidant screening^{a,b}

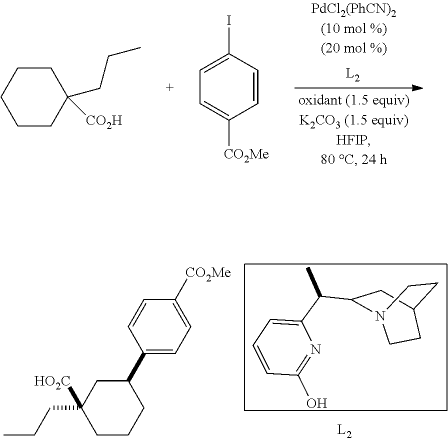

| entry | oxidant | yield (%) |
|---|---|---|
| 1 | Ag₂CO₃ | 65 |
| 2 | Ag₂O | 28 |
| 3 | AgNO₃ | 31 |
| 4 | AgPO₄ | 35 |
| 5 | AgTFA | 44 |
| 6 | AgOAc | 33 |
| 7 | Na₂CO₃•1.5H₂O₂ | 10 |

^{a}Conditions: substrate (0.1 mmol), methyl 4-iodobenzoate 2a (0.2 mmol), PdCl₂(PhCN)₂ (10 mol %), L2 (20 mol %), oxidant (1.5 equiv), K₂CO₃ (1.5 equiv), HFIP (0.6 mL), 80 °C, 24 h.
^{b}yields determined by ¹H NMR using dibromomethane as internal standard

TABLE S12
Temperature and concentration screening^{a,b}

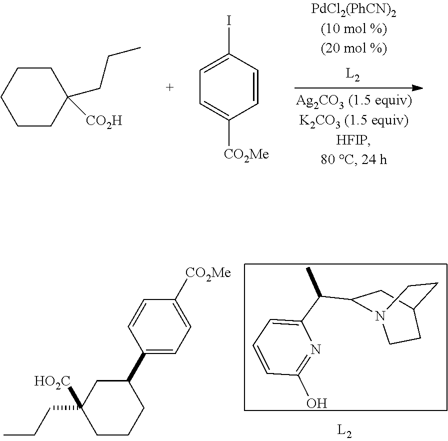

| entry | T °C^{a} | yield (%) |
|---|---|---|
| 1 | 120 | 54 |
| 2 | 100 | 53 |
| 3 | 90 | 65 |
| 4 | 80 | 61 |
| 5 | 70 | 51 |
| 6 | 60 | 30 |
| 7 | 0.2 | 11 |
| 8 | 0.4 | 57 |
| 9 | 0.8 | 66 |
| 10 | 1.0 | 70 |

^{a}Conditions: substrate (0.1 mmol), methyl 4-iodobenzoate 2a (0.2 mmol), PdCl₂(PhCN)₂ (10 mol %), L2 (20 mol %), oxidant (1.5 equiv), K₂CO₃ (1.5 equiv), HFIP (× mL), T °C, 24 h.
^{b}yields determined by ¹H NMR using dibromomethane as internal standard

TABLE S13
Amounts of reagents screening^{a,b}

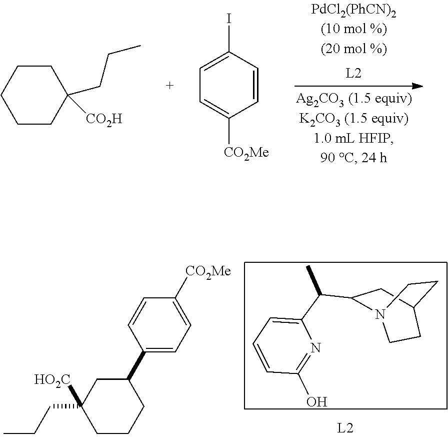

| entry | virants | yield (%)^{a} |
|---|---|---|
| 1 | no change | 70 (65)^{b} |
| 2 | 1.5 equiv Arl | 62 |
| 7 | 5 mol % PdCl_{₂}_{(PhCN)}_{₂}_{,} 10 mol % Ligand | 9 |
| 8 | 10 mol % PdCl_{₂}_{(PhCN)}_{₂}, 10 mol % Ligand | 31 |
| 9 | 10 mol % PdCl_{₂}_{(PhCN)}_{₂}_{,} 15 mol % Ligand | 29 |

^{a}Conditions: substrate (0.1 mmol), methyl 4-iodobenzoate 2a (0.2 mmol), PdCl₂(PhCN)₂ (10 mol %), L2 (20 mol %), Ag₂CO₃ (1.5 equiv), K₂CO₃ (1.5 equiv), HFIP (1.0 mL), 90 °C, 24 h, all with variation as indicated above. Yields determined by ¹H NMR using dibromomethane as internal standard.
^{b}Isolated yields

Optimization for Transannular γ-C—H Arylation of α-propyl Cycloheptane Carboxylic Acid

TABLE S14
Condition screening for α-propyl cycloheptane carboxylic acid^{a,b}

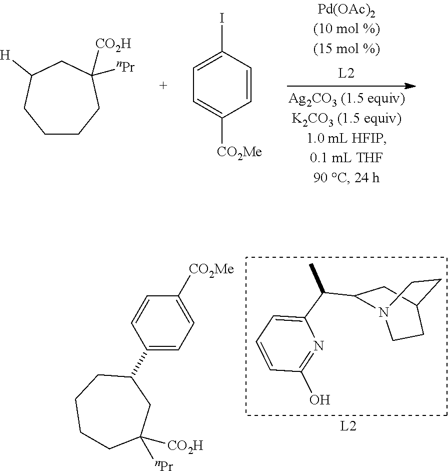

| entry | variant | yield (%) |
|---|---|---|
| 1 | MeCN 100 uL | 26 |
| 2 | THF 100 uL | 44 |
| 3 | ^{Dioxane 100 uL} | 30 |
| 4 | THF 50 uL | 18 |
| 5 | THF 150 uL | 40 |
| 6 | 100 °C | 30 |
| 7 | 80 °C | 40 |
| 8 | Ag₂CO₃^{(2 equiv.)} | 26 |
| 9 | 13% Lignad | 36 |
| 10 | 20% ligand | 32 |

^{a}Unless otherwise notified, the conditions will be as follows: substrate (0.1 mmol), methyl 4-iodobenzoate 2a (0.2 mmol), Pd(OAc)₂ (10 mol %), L2 (15 mol %), Ag₂CO₃ (1.5 equiv), K₂CO₃ (1.5 equiv), HFIP (1.0 mL), 0.1 mL THF, 90 °C, 24 h.
^{b}yields determined by ¹H NMR using dibromomethane as internal standard.

TABLE S15
Base screening for α-propyl cycloheptane carboxylic acid^{a,b}

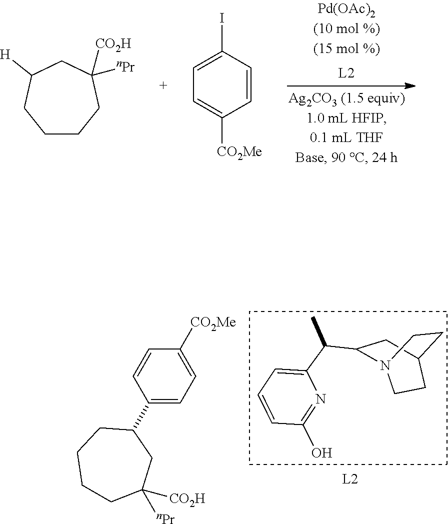

| entry | Base | yield (%) |
|---|---|---|
| 1 | 1.5 eq K₂CO₃ | 44 |
| 2 | 2 eq K₂CO₃ | 48 |
| 3 | 3 eq K₂CO₃ | 53 (52)^{c} |
| 4 | 4 eq K₂CO₃ | 52 |
| 5 | 2 eq K₃PO₄ | 34 |
| 6 | 2 eq K₂HPO₄ | 34 |
| 7 | 2 eq KH₂PO₄ | 20 |
| 8 | 2 eq KF | 26 |
| 9 | 2 eq LiCO₃ | 20 |
| 10 | 2 eq Cs₂CO₃ | 38 |
| 11 | 2 eq t-BuOK | 26 |

^{a}Unless otherwise notified, the conditions will be as follows: substrate (0.1 mmol), methyl 4-iodobenzoate 2a (0.2 mmol), Pd(OAc)₂ (10 mol %), L2 (15 mol %), Ag₂CO₃ (1.5 equiv), Base, HFIP (1.0 mL), 0.1 mL THF, 90 °C, 24 h.
^{b}yields determined by ¹H NMR using dibromomethane as internal standard.
^{c}Isolated yield.

Optimization for Transannular γ-C—H Arylation of α-propyl Cyclooctane Carboxylic Acid

TABLE S16
Ligand, temperature and base screening for α-propyl cyclooctane
carboxylic acid^{a,b}

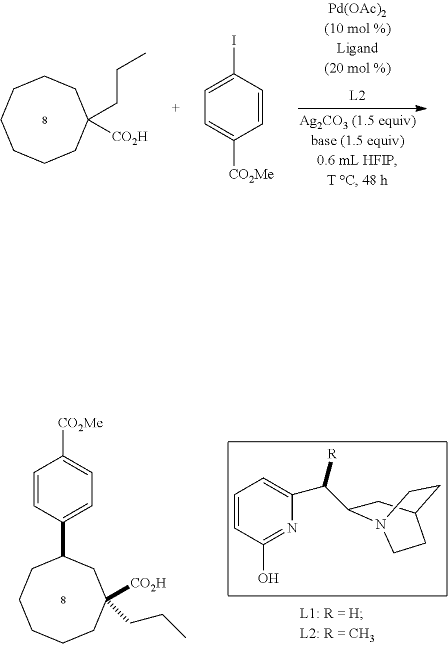

| entry | T °C | Ligand | Base | yield (%) |
|---|---|---|---|---|
| 1 | 80 | L2 | Na₂CO₃ | 13 |
| 2 | 80 | L1 | Na₂CO₃ | 32 |
| 3 | 100 | L1 | Na₂CO₃ | 34 |
| 4 | 120 | L1 | Na₂CO₃ | 35 |
| 5 | 120 | L1 | K₂CO₃ | 22 |
| 6 | 120 | L1 | Cs₂CO₃ | 29 |
| 7 | 100 | L1 | Li₂CO₃ | 18 |
| 8 | 120 | L1 | Na₃PO₄ | 41 |
| 9 | 120 | L1 | K₂HPO₄ | 33 |
| 10 | 120 | L1 | t-BuOK | 36 |

^{a}Unless otherwise notified, the conditions will be as follows: substrate (0.1 mmol), methyl 4-iodobenzoate 2a (0.2 mmol), Pd(OAc)₂ (10 mol %), ligand (20 mol %), Ag₂CO₃ (1.5 equiv), Base (1.5 equiv.), HFIP (0.6 mL), T °C, 48 h.
^{b}yields determined by ¹H NMR using dibromomethane as internal standard.

TABLE S17
Additive and amount screening for α-propyl cyclooctane carboxylic acid^{a,b}

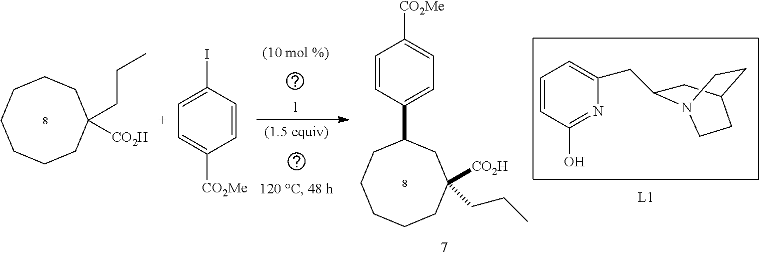

| entry | additive | L1₍ₓ₎ | Na₃PO₄⁽⁷⁾ | yield (%) |
|---|---|---|---|---|
| 1 | 0.1 mL THF | 20 | 1.5 | 40 |
| 2 | 0.1 mL CH₃CN | 20 | 1.5 | 42 |
| 3 | — | 15 | 1.5 | 44 (40)^{c} |
| 4 | — | 20 | 2.0 | 35 |
| 5 | — | 20 | 3.0 | 22 |

^{a}Unless otherwise notified, the conditions will be as follows: substrate (0.1 mmol), methyl 4-iodobenzoate 2a (0.2 mmol), Pd(OAc)₂ (10 mol %), L1 (× mol %), Ag₂CO₃ (1.5 equiv), Na₃PO₄ (y equiv), HFIP (0.6 mL), 120 °C, 48 h.
^{b}yields determined by ¹H NMR using dibromomethane as internal standard.
^{c}Isolated yield.

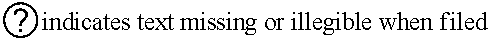

General Procedure for Transannular γ-methylene-C(sp³)-H Arylation:

### General Procedure 1

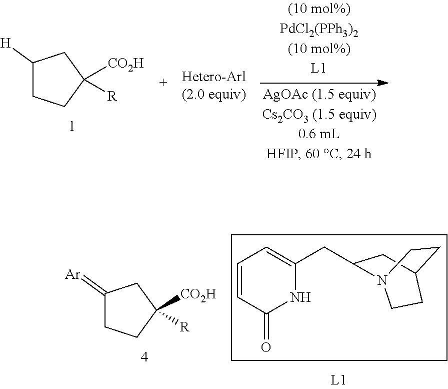

A sealed tube equipped with a magnetic stir bar was charged with PdCl₂(PPh₃)₂ (7.0 mg, 10 mol %), L1 (2.2 mg, 10 mol %), the appropriate carboxylic acid substrate (0.10 mmol), (hetero)aryliodide (0.2 mmol), AgOAc (25 mg, 0.15 mmol), and Cs₂CO₃ (48.9 mg, 0.15 mmol). HFIP (0.6 mL) was then added. The reaction mixture was then stirred at the rate of 300 rpm at 60° C. for 24 h. After being allowed to cool to room temperature, the mixture was diluted with ethyl acetate and acidified with 0.05 mL of formic acid. The mixture was passed through a pad of Celite with acetone as the eluent to remove any insoluble precipitate. The resulting solution was concentrated, and the residual mixture was isolated using pTLC (hexane:ethyl acetate=4:1 to 0:1).

### General Procedure 2

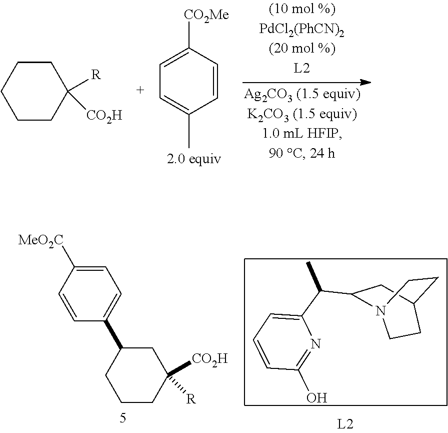

A sealed tube equipped with a magnetic stir bar was charged with PdCl₂(PhCN)₂ (3.8 mg, 10 mol %), L2 (4.6 mg, 20 mol %), the appropriate carboxylic acid substrate (0.10 mmol), methyl 4-iodobenzoate (52.4 mg, 0.2 mmol), Ag₂CO₃ (41.4 mg, 0.15 mmol), and K₂CO₃ (20.7 mg, 0.15 mmol). HFIP (1 mL) was then added. The reaction mixture was then stirred at the rate of 300 rpm at 90° C. for 24 h. After being allowed to cool to room temperature, the mixture was diluted with ethyl acetate and acidified with 0.05 mL of formic acid. The mixture was passed through a pad of Celite with acetone as the eluent to remove any insoluble precipitate. The resulting solution was concentrated, and the residual mixture was isolated using pTLC (hexane:ethyl acetate=4:1 to 1:1).

### General Procedure 3

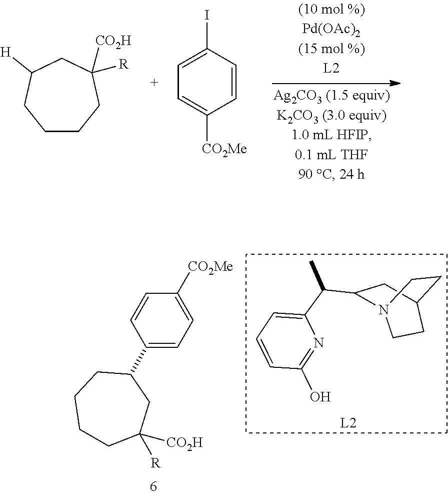

A sealed tube equipped with a magnetic stir bar was charged with Pd(OAc)₂ (2.2 mg, 10 mol %), L2 (3.5 mg, 15 mol %), the appropriate carboxylic acid substrate (0.10 mmol), methyl 4-iodobenzoate (52.4 mg, 0.2 mmol), Ag₂CO₃ (41.4 mg, 0.15 mmol), and K₂CO₃ (41.4 mg, 0.30 mmol). HFIP (1 mL) and THE (0.1 mL) were then added. The reaction mixture was then stirred at the rate of 300 rpm at 90° C. for 24 h. After being allowed to cool to room temperature, the mixture was diluted with ethyl acetate and acidified with 0.05 mL of formic acid. The mixture was passed through a pad of Celite with acetone as the eluent to remove any insoluble precipitate. The resulting solution was concentrated, and the residual mixture was isolated using pTLC (hexane:ethyl acetate=4:1 to 1:1).

### General Procedure 4

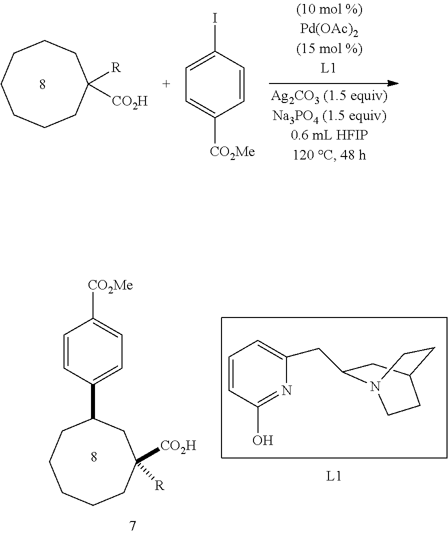

A sealed tube equipped with a magnetic stir bar was charged with Pd(OAc)₂ (2.2 mg, 10 mol %), Li (3.3 mg, 15 mol %), the appropriate carboxylic acid substrate (0.10 mmol), methyl 4-iodobenzoate (52.4 mg, 0.2 mmol), Ag₂CO₃ (41.4 mg, 0.15 mmol), and Na₃PO₄ (24.6 mg, 0.15 mmol). HFIP (0.6 mL) was then added. The reaction mixture was then stirred at the rate of 300 rpm at 120° C. for 48 h. After being allowed to cool to room temperature, the mixture was diluted with ethyl acetate and acidified with 0.05 mL of formic acid. The mixture was passed through a pad of Celite with acetone as the eluent to remove any insoluble precipitate. The resulting solution was concentrated, and the residual mixture was isolated using pTLC (hexane:ethyl acetate=4:1 to 2:1).

Characterization Data of Products Obtained from Transannular γ-Methylene C—H Arylation

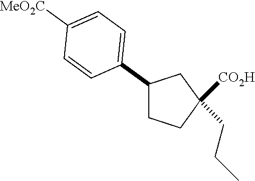 [SMILES: CCC[C@]1(C(=O)O)CC[C@@H](c2ccc(C(=O)OC)cc2)C1]

### cis-3-(4-(methoxycarbonyl)phenyl)-1-propylcyclopentane-1-carboxylic acid (3a)

Following General Procedure 1 on 0.1 mmol scale. The product was obtained as colorless oil (25.3 mg, 87% yield). ¹H NMR (600 MHz, CDCl₃) δ 7.98-7.93 (m, 2H), 7.34-7.29 (m, 2H), 3.90 (s, 3H), 3.18 (tt, J=11.0, 7.5 Hz, 1H), 2.43 (ddd, J=13.2, 7.8, 2.5 Hz, 1H), 2.24 (dd, J=13.6, 10.8 Hz, 1H), 2.16-2.06 (m, 2H), 1.84-1.63 (m, 4H), 1.40-1.30 (m, 2H), 0.94 (t, J=7.3 Hz, 3H); ¹³C NMR (151 MHz, CDCl₃) δ 184.3, 167.3, 150.2, 129.9, 128.2, 127.3, 53.6, 52.1, 45.1, 43.4, 42.1, 36.5, 34.4, 19.1, 14.7; HRMS (ESI-TOF) m/z Calcd for C₁₇H₂₃O₄⁺ [M+H]⁺ 291.1596, found 291.1604.

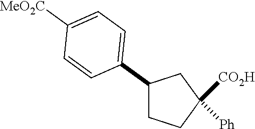 [SMILES: COC(=O)c1ccc([C@@H]2CC[C@@](C(=O)O)(c3ccccc3)C2)cc1]

### cis-3-(4-(methoxycarbonyl)phenyl)-1-phenylcyclopentane-1-carboxylic acid (3b)

Following General Procedure 1 on 0.1 mmol scale. The product was obtained as colorless oil (26.2 mg, 81% yield). ¹H NMR (600 MHz, CDCl₃) δ 7.98-7.94 (m, 2H), 7.46-7.42 (m, 2H), 7.39-7.35 (m, 2H), 7.34-7.31 (m, 2H), 7.31-7.27 (m, 1H), 3.90 (s, 3H), 3.26-3.16 (m, 1H), 2.81 (ddd, J=13.0, 7.8, 4.0 Hz, 1H), 2.71 (dd, J=9.3, 1.9 Hz, 2H), 2.31 (ddd, J=13.0, 9.3, 7.0 Hz, 1H), 2.25-2.17 (m, 1H), 1.87-1.80 (m, 1H); ¹³C NMR (151 MHz, CDCl₃) δ 181.4, 167.2, 150.6, 142.5, 129.9, 128.7, 128.2, 127.4, 127.3, 126.9, 58.4, 52.2, 44.3, 43.7, 36.6, 33.7; HRMS (ESI-TOF) m/z Calcd for C₂₀H₂₁O₄⁺ [M+H]⁺ 325.1440, found 325.1443.

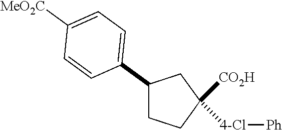

### cis-1-(4-chlorophenyl)-3-(4-(methoxycarbonyl)phenyl)cyclopentane-1-carboxylic acid (3c)

Following General Procedure 1 on 0.1 mmol scale. The product was obtained as colorless oil (31.9 mg, 89% yield). ¹H NMR (600 MHz, CDCl₃) δ 7.98-7.94 (m, 2H), 7.39-7.30 (m, 6H), 3.90 (s, 3H), 3.25-3.15 (m, 1H), 2.83-2.76 (m, 1H), 2.71 (dd, J=13.7, 9.9 Hz, 1H), 2.64 (dd, J=13.7, 8.8 Hz, 1H), 2.30-2.17 (m, 2H), 1.90-1.79 (m, 1H); ¹³C NMR (151 MHz, CDCl₃) δ 181.0, 167.2, 150.3, 141.0, 133.4, 130.0, 128.9, 128.4, 128.3, 127.3, 58.0, 52.2, 44.2, 43.8, 36.7, 33.6; HRMS (ESI-TOF) m/z Calcd for C₂₀H₂₀ClO₄⁺ [M+H]⁺ 359.1050, found 359.1064.

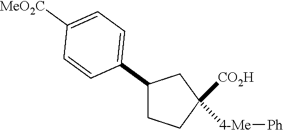

### cis-3-(4-(methoxycarbonyl)phenyl)-1-(p-tolyl)cyclopentane-1-carboxylic acid (3d)

Following General Procedure 1 on 0.1 mmol scale. The product was obtained as colorless oil (25.7 mg, 76% yield). ¹H NMR (600 MHz, CDCl₃) δ 7.99-7.93 (m, 2H), 7.36-7.30 (m, 4H), 7.21-7.15 (m, 2H), 3.90 (s, 3H), 3.24-3.16 (m, 1H), 2.77 (ddd, J=12.5, 7.9, 4.2 Hz, 1H), 2.68 (d, J=9.3 Hz, 2H), 2.35 (s, 3H), 2.30 (ddd, J=13.0, 9.1, 7.0 Hz, 1H), 2.22-2.17 (m, 1H), 1.87-1.77 (m, 1H); ¹³C NMR (151 MHz, CDCl₃) δ 181.4, 167.2, 150.7, 139.5, 137.1, 129.9, 129.4, 128.20, 127.3, 126.8, 58.1, 52.1, 44.3, 43.8, 36.6, 33.6, 21.1; HRMS (ESI-TOF) m/z Calcd for C₂₁H₂₃O₄⁺ [M+H]339.1596, found 339.1609.

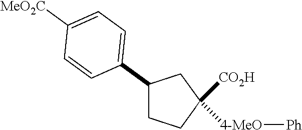

### cis-3-(4-(methoxycarbonyl)phenyl)-1-(4-methoxyphenyl)cyclopentane-1-carboxylic acid (3e)

Following General Procedure 1 on 0.1 mmol scale. The product was obtained as colorless oil (30 mg, 85% yield). ¹H NMR (600 MHz, CDCl₃) δ 7.99-7.93 (m, 2H), 7.38-7.34 (m, 2H), 7.34-7.30 (m, 2H), 6.93-6.86 (m, 2H), 3.90 (s, 3H), 3.81 (s, 3H), 3.26-3.15 (m, 1H), 2.77 (ddd, J=12.5, 8.0, 4.2 Hz, 1H), 2.67 (dd, J=9.3, 3.7 Hz, 2H), 2.28 (ddd, J=12.9, 9.1, 7.1 Hz, 1H), 2.24-2.16 (m, 1H), 1.85-1.78 (m, 1H); ¹³C NMR (151 MHz, CDCl₃) δ 181.5, 167.2, 158.8, 150.7, 134.5, 129.9, 128.1, 127.3, 114.1, 57.7, 55.4, 52.2, 44.2, 43.8, 36.6, 33.6; HRMS (ESI-TOF) m/z Calcd for C₂₁H₂₃O₅⁺ [M+H]⁺ 355.1545, found 355.1555.

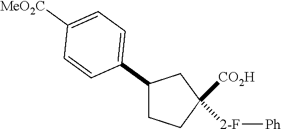

### cis-1-(2-fluorophenyl)-3-(4-(methoxycarbonyl)phenyl)cyclopentane-1-carboxylic acid (3f)

Following General Procedure 1 on 0.1 mmol scale. The product was obtained as colorless oil (21.9 mg, 64% yield). ¹H NMR (600 MHz, CDCl₃) δ 8.00-7.94 (m, 2H), 7.41 (td, J=7.9, 1.7 Hz, 1H), 7.38-7.35 (m, 2H), 7.32-7.27 (m, 1H), 7.17 (td, J=7.6, 1.3 Hz, 1H), 7.08 (ddd, J=11.1, 8.1, 1.2 Hz, 1H), 3.90 (s, 3H), 3.22 (tt, J=10.6, 7.9 Hz, 1H), 2.77-2.68 (m, 2H), 2.62 (dd, J=13.8, 8.0 Hz, 1H), 2.36 (ddd, J=13.5, 9.0, 7.3 Hz, 1H), 2.27-2.21 (m, 1H), 2.01-1.95 (m, 1H); ¹³C NMR (151 MHz, CDCl₃) δ 181.4, 167.2, 160.97 (d, J=247.0 Hz), 150.0, 130.72 (d, J=14.2 Hz), 130.0, 129.13 (d, J=8.7 Hz), 129.1, 128.4, 127.14 (d, J=4.4 Hz), 124.10 (d, J=3.4 Hz), 116.14 (d, J=22.0 Hz), 54.9, 52.2, 44.9, 43.8, 36.7, 33.7; HRMS (ESI-TOF) m/z Calcd for C₂₀H₂₀FO₄⁺[M+H]⁺ 343.1346, found 343.1356.

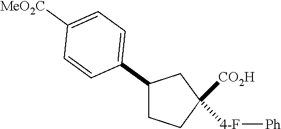

### cis-1-(4-fluorophenyl)-3-(4-(methoxycarbonyl)phenyl)cyclopentane-1-carboxylic acid (3g)

Following General Procedure 1 on 0.1 mmol scale. The product was obtained as colorless oil (30.8 mg, 90% yield). ¹H NMR (600 MHz, CDCl₃) δ 8.00-7.92 (m, 2H), 7.45-7.37 (m, 2H), 7.35-7.30 (m, 2H), 7.09-7.02 (m, 2H), 3.90 (s, 3H), 3.25-3.16 (m, 1H), 2.80 (ddd, J=12.1, 7.8, 3.8 Hz, 1H), 2.72 (dd, J=13.7, 9.8 Hz, 1H), 2.65 (dd, J=13.7, 8.8 Hz, 1H), 2.31-2.16 (m, 2H), 1.87-1.80 (m, 1H); ¹³C NMR (151 MHz, CDCl₃) δ 181.3, 167.2, 162.04 (d, J=246.5 Hz), 150.4, 138.20 (d, J=3.3 Hz), 130.0, 128.7, 128.65 (d, J=7.8 Hz), 127.3, 115.52 (d, J=21.4 Hz), 57.9, 52.2, 44.2, 43.9, 36.8, 33.6; HRMS (ESI-TOF) m/z Calcd for C₂₀H₂₀FO₄⁺ [M+H]⁺ 343.1346, found 343.1360.

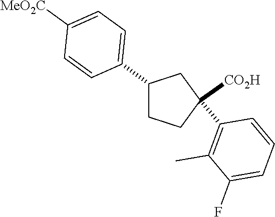 [SMILES: COC(=O)c1ccc([C@H]2CC[C@@](C(=O)O)(c3cccc(F)c3C)C2)cc1]

### cis-1-(3-fluoro-2-methylphenyl)-3-(4-(methoxycarbonyl)phenyl)cyclopentane-1-carboxylic acid (3h)

Following General Procedure 1 on 0.1 mmol scale. The product was obtained as colorless oil (29.2 mg, 82% yield). ¹H NMR (600 MHz, CDCl₃) δ 8.00-7.94 (m, 2H), 7.38 (d, J=8.3 Hz, 2H), 7.24-7.16 (m, 2H), 6.99 (ddd, J=9.3, 7.0, 2.3 Hz, 1H), 3.91 (s, 3H), 3.23 (tt, J=10.8, 7.8 Hz, 1H), 2.78-2.69 (m, 2H), 2.55 (dd, J=13.5, 7.9 Hz, 1H), 2.39 (ddd, J=13.4, 9.6, 7.2 Hz, 1H), 2.27-2.19 (m, 1H), 2.17 (s, 3H), 2.01-1.91 (m, 1H); ¹³C NMR (151 MHz, CDCl₃) δ 182.1, 167.2, 162.18 (d, J=243.7 Hz), 149.8, 143.95 (d, J=3.4 Hz), 130.0, 128.4, 127.4, 126.76 (d, J=9.2 Hz), 124.00 (d, J=16.0 Hz), 121.25 (d, J=3.2 Hz), 114.15 (d, J=23.7 Hz), 57.70 (d, J=2.5 Hz), 52.2, 45.2, 44.6, 38.0, 33.7, 12.20 (d, J=7.1 Hz); HRMS (ESI-TOF)m/z Calcd for Chemical Formula: C₂₁H₂₂FO₄⁺[M+H]357.1502, found 357.1492.

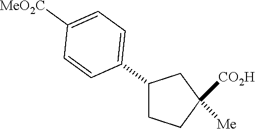 [SMILES: COC(=O)c1ccc([C@H]2CC[C@](C)(C(=O)O)C2)cc1]

### cis-3-(4-(methoxycarbonyl)phenyl)-1-methylcyclopentane-1-carboxylic acid (3i)

Following General Procedure 1 on 0.1 mmol scale. The product was obtained as colorless oil (18.1 mg, 69% yield). ¹H NMR (600 MHz, CDCl₃) δ 7.99-7.94 (m, 2H), 7.36-7.31 (m, 2H), 3.90 (s, 3H), 3.27 (tt, J=11.0, 7.3 Hz, 1H), 2.44 (ddd, J=13.3, 8.6, 3.3 Hz, 1H), 2.30 (dd, J=13.1, 11.2 Hz, 1H), 2.20-2.03 (m, 1H), 2.04 (ddd, J=13.1, 7.5, 1.3 Hz, 1H), 1.90-1.83 (m, 1H), 1.67 (ddd, J=13.3, 9.8, 7.3 Hz, 1H), 1.40 (s, 3H); ¹³C NMR (151 MHz, CDCl₃) δ 184.6, 167.2, 150.2, 129.9, 128.3, 127.3, 52.2, 48.9, 45.6, 45.1, 38.4, 34.6, 25.6; HRMS (ESI-TOF) m/z Calcd for C₁₅H₁₉O₄⁺[M+H]⁺ 263.1283, found 263.1294.

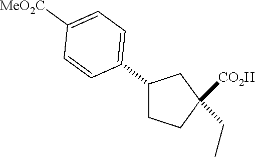 [SMILES: CC[C@]1(C(=O)O)CC[C@H](c2ccc(C(=O)OC)cc2)C1]

### cis-1-ethyl-3-(4-(methoxycarbonyl)phenyl)cyclopentane-1-carboxylic acid (3j)

Following General Procedure 1 on 0.1 mmol scale. The product was obtained as colorless oil (22 mg, 80% yield). ¹H NMR (500 MHz, CDCl₃) δ 8.01-7.91 (m, 2H), 7.32 (d, J=8.2 Hz, 2H), 3.90 (s, 3H), 3.17 (tt, J=10.9, 7.3 Hz, 1H), 2.42 (ddd, J=13.2, 7.7, 2.4 Hz, 1H), 2.24 (dd, J=13.5, 10.9 Hz, 1H), 2.12 (dt, J=15.2, 7.6 Hz, 2H), 1.84-1.75 (m, 3H), 1.67 (ddd, J=13.1, 10.8, 6.8 Hz, 1H), 0.95 (t, J=7.3 Hz, 3H); ¹³C NMR (126 MHz, CDCl₃) δ 184.0, 167.3, 150.2, 129.9, 128.2, 127.3, 54.1, 52.1, 45.1, 43.0, 36.1, 34.5, 32.4, 10.1; HRMS (ESI-TOF) m/z Calcd for C₁₆H₂₁O₄⁺[M+H]⁺ 277.1440, found 277.1441.

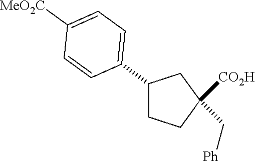 [SMILES: COC(=O)c1ccc([C@H]2CC[C@](Cc3ccccc3)(C(=O)O)C2)cc1]

### cis-1-benzyl-3-(4-(methoxycarbonyl)phenyl)cyclopentane-1-carboxylic acid (3k)

Following General Procedure 1 on 0.1 mmol scale. The product was obtained as colorless oil (15.2 mg, 45% yield). ¹H NMR (600 MHz, CDCl₃) δ 7.97-7.93 (m, 2H), 7.32-7.26 (m, 4H), 7.26-7.21 (m, 3H), 3.90 (s, 3H), 3.19 (tt, J=10.4, 7.6 Hz, 1H), 3.11 (d, J=2.5 Hz, 2H), 2.37 (ddd, J=12.1, 7.3, 2.6 Hz, 1H), 2.32-2.21 (m, 2H), 2.20-2.14 (m, 1H), 1.91-1.77 (m, 2H); ¹³C NMR (151 MHz, CDCl₃) δ 183.1, 167.2, 150.2, 138.0, 129.9, 129.9, 128.5, 128.3, 127.3, 126.9, 54.8, 52.2, 44.8, 44.3, 42.8, 36.1, 34.1; HRMS (ESI-TOF) m/z Calcd for C₂₁H₂₃₀₄+[M+H]⁺ 339.1596, found 339.1610.

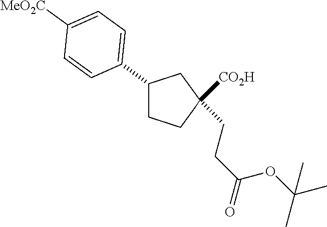 [SMILES: COC(=O)c1ccc([C@H]2CC[C@](CCC(=O)OC(C)(C)C)(C(=O)O)C2)cc1]

### cis-1-(3-(tert-butoxy)-3-oxopropyl)-3-(4-(methoxycarbonyl)phenyl)cyclopentane-1-carboxylic acid (31)

Following General Procedure 1 on 0.1 mmol scale. The product was obtained as colorless oil (33.1 mg, 88% yield). ¹H NMR (600 MHz, CDCl₃) δ 7.99-7.92 (m, 2H), 7.35-7.29 (m, 2H), 3.90 (s, 3H), 3.22 (tt, J=11.1, 7.3 Hz, 1H), 2.42 (ddd, J=13.3, 7.9, 2.5 Hz, 1H), 2.33-2.22 (m, 3H), 2.17-1.99 (m, 4H), 1.88-1.78 (m, 1H), 1.69 (ddd, J=13.2, 10.6, 7.0 Hz, 1H), 1.45 (s, 9H); ¹³C NMR (151 MHz, CDCl₃) δ 183.3, 172.7, 167.2, 149.8, 129.9, 128.3, 127.3, 80.7, 52.7, 52.2, 45.1, 43.5, 36.5, 34.4, 34.0, 32.1, 28.2; HRMS (ESI-TOF) m/z Calcd for C₂₁H₂₈O₆Na⁺ [M+Na]⁺ 399.1784, found 399.1776.

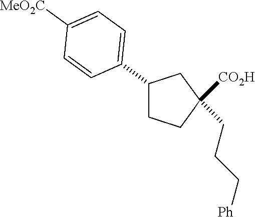 [SMILES: COC(=O)c1ccc([C@H]2CC[C@](CCCc3ccccc3)(C(=O)O)C2)cc1]

### cis-3-(4-(methoxycarbonyl)phenyl)-1-(3-phenylpropyl)cyclopentane-1-carboxylic acid (3m)

Following General Procedure 1 on 0.1 mmol scale. The product was obtained as colorless oil (22.7 mg, 62% yield). ¹H NMR (600 MHz, CDCl₃) δ 7.98-7.94 (m, 2H), 7.33-7.27 (m, 4H), 7.21-7.17 (m, 3H), 3.90 (s, 3H), 3.15 (tt, J=11.0, 7.3 Hz, 1H), 2.65 (t, J=7.6 Hz, 2H), 2.42 (ddd, J=13.2, 7.8, 2.4 Hz, 1H), 2.25 (dd, J=13.5, 10.9 Hz, 1H), 2.15-2.06 (m, 2H), 1.84-1.74 (m, 3H), 1.72-1.61 (m, 3H); ¹³C NMR (151 MHz, CDCl₃) δ 184.0, 167.2, 150.1, 142.1, 129.9, 128.5, 128.5, 128.2, 127.3, 126.0, 53.4, 52.1, 45.1, 43.4, 39.2, 36.6, 36.3, 34.4, 27.5; HRMS (ESI-TOF) m/z Calcd for C₂₃H₂₇₀₄⁺[M+H]367.1909, found 367.1924.

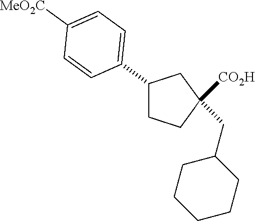 [SMILES: COC(=O)c1ccc([C@H]2CC[C@](CC3CCCCC3)(C(=O)O)C2)cc1]

### cis-1-(cyclohexylmethyl)-3-(4-(methoxycarbonyl)phenyl)cyclopentane-1-carboxylic acid (3n)

Following General Procedure 1 on 0.1 mmol scale. The product was obtained as colorless oil (20.0 mg, 58% yield). ¹H NMR (600 MHz, CDCl₃) δ 7.98-7.93 (m, 2H), 7.35-7.29 (m, 2H), 3.90 (s, 3H), 3.18 (tt, J=10.7, 7.8 Hz, 1H), 2.44 (ddd, J=12.9, 7.3, 2.7 Hz, 1H), 2.25 (dd, J=13.5, 10.6 Hz, 1H), 2.18-2.06 (m, 2H), 1.80-1.58 (m, 9H), 1.41-1.34 (m, 1H), 1.24-1.07 (m, 3H), 1.03-0.92 (m, 2H); ¹³C NMR (151 MHz, CDCl₃) δ 184.4, 167.3, 150.4, 129.9, 128.2, 127.3, 53.0, 52.1, 47.0, 44.9, 44.1, 37.1, 35.6, 34.3, 34.2, 34.1, 26.5, 26.5, 26.4; HRMS (ESI-TOF) m/z Calcd for C₂₁H₂₉O₄⁺[M+H]⁺ 345.2066, found 345.2064.

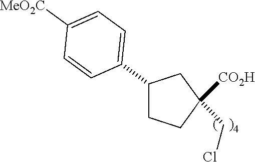

### cis-1-(4-chlorobutyl)-3-(4-(methoxycarbonyl)phenyl)cyclopentane-1-carboxylic acid (3o)

Following General Procedure 1 on 0.1 mmol scale. The product was obtained as colorless oil (20.9 mg, 62% yield). ¹H NMR (600 MHz, CDCl₃) δ 7.99-7.93 (m, 2H), 7.35-7.30 (m, 2H), 3.90 (s, 3H), 3.56 (t, J=6.6 Hz, 2H), 3.19 (tt, J=11.1, 7.3 Hz, 1H), 2.44 (ddd, J=13.1, 7.7, 2.3 Hz, 1H), 2.26 (dd, J=13.6, 10.9 Hz, 1H), 2.17-2.09 (m, 2H), 1.87-1.64 (m, 6H), 1.53-1.46 (m, 2H); ¹³C NMR (151 MHz, CDCl₃) δ 183.7, 167.2, 150.0, 129.9, 128.3, 127.3, 53.4, 52.2, 45.2, 44.8, 43.5, 38.8, 36.6, 34.5, 33.0, 23.2; HRMS (ESI-TOF) m/z Calcd for C₁₈H₂₄C₁₀₄⁺[M+H]⁺ 339.1363, found 339.1368.

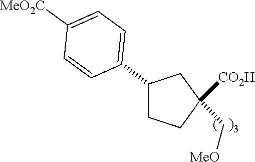

### cis-3-(4-(methoxycarbonyl)phenyl)-1-(3-methoxypropyl)cyclopentane-1-carboxylic acid (3p)

Following General Procedure 1 on 0.1 mmol scale. The product was obtained as colorless oil (23.9 mg, 70% yield). ¹H NMR (600 MHz, CDCl₃) δ 7.97-7.93 (m, 2H), 7.34-7.29 (m, 2H), 3.90 (s, 3H), 3.41 (t, J=6.3 Hz, 2H), 3.34 (s, 3H), 3.19 (tt, J=11.0, 7.4 Hz, 1H), 2.43 (ddd, J=13.2, 7.8, 2.4 Hz, 1H), 2.26 (dd, J=13.6, 10.9 Hz, 1H), 2.16-2.08 (m, 2H), 1.87-1.76 (m, 3H), 1.72-1.58 (m, 3H); ¹³C NMR (151 MHz, CDCl₃) δ 183.3, 167.2, 150.1, 129.9, 128.2, 127.3, 72.9, 58.7, 53.2, 52.1, 45.1, 43.5, 36.6, 36.2, 34.5, 26.1; HRMS (ESI-TOF) m/z Calcd for C₁₈H₂₄O₅Na⁺[M+H]⁺ 343.1521, found 343.1521.

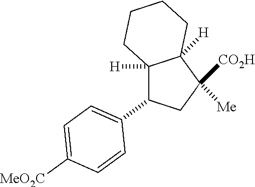 [SMILES: COC(=O)c1ccc([C@H]2C[C@](C)(C(=O)O)[C@@H]3CCCC[C@H]23)cc1]

### rel-(1S,3R,3aR,7aR)-3-(4-(methoxycarbonyl)phenyl)-1-methyloctahydro-1H-indene-1-carboxylic acid (3q)

Following General Procedure 1 on 0.1 mmol scale. The product was obtained as colorless oil (10.7 mg, 34% yield). ¹H NMR (600 MHz, CDCl₃) δ 7.98-7.94 (m, 2H), 7.38-7.34 (m, 2H), 3.89 (s, 3H), 3.27 (td, J=11.3, 7.6 Hz, 1H), 2.68 (dd, J=14.5, 7.6 Hz, 1H), 2.40 (dt, J=11.2, 5.3 Hz, 1H), 2.26-2.20 (m, 1H), 2.05 (dd, J=14.5, 10.8 Hz, 1H), 1.84 (d, J=12.0 Hz, 1H), 1.69 (dd, J=9.8, 5.1 Hz, 1H), 1.46-1.35 (m, 3H), 1.32 (s, 3H), 1.27-1.15 (m, 4H); ¹³C NMR (151 MHz, CDCl₃) δ 184.6, 167.3, 151.1, 129.9, 128.1, 128.1, 52.6, 52.1, 47.7, 47.2, 44.9, 42.5, 25.7, 24.3, 24.1, 21.0, 20.7; HRMS (ESI-TOF) m/z Calcd for C₁₉H₂₅O₄⁺[M+H]⁺ 317.1753, found 317.1764.

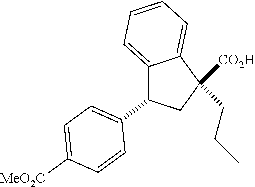 [SMILES: CCC[C@]1(C(=O)O)C[C@H](c2ccc(C(=O)OC)cc2)c2ccccc21]

### cis-3-(4-(methoxycarbonyl)phenyl)-1-propyl-2,3-dihydro-1H-indene-1-carboxylic acid (3r)

Following General Procedure 1 on 0.1 mmol scale. The product was obtained as colorless oil (6.8 mg, 22% yield). ¹H NMR (600 MHz, CDCl₃) δ 8.01-7.97 (m, 2H), 7.55 (dd, J=7.7, 1.1 Hz, 1H), 7.31-7.26 (m, 3H), 7.21 (td, J=7.4, 1.2 Hz, 1H), 6.89-6.84 (m, 1H), 4.44 (t, J=8.8 Hz, 1H), 3.91 (s, 3H), 2.72 (dd, J=13.5, 9.4 Hz, 1H), 2.63 (dd, J=13.5, 8.2 Hz, 1H), 2.02-1.93 (m, 1H), 1.82 (ddd, J=13.5, 12.1, 4.6 Hz, 1H), 1.46-1.27 (m, 3H), 0.92 (t, J=7.3 Hz, 3H); ¹³C NMR (151 MHz, CDCl₃) δ 180.8, 167.2, 150.1, 145.4, 144.9, 130.1, 128.7, 128.6, 128.2, 127.4, 125.3, 124.9, 58.3, 52.2, 49.3, 44.1, 41.3, 19.0, 14.5; HRMS (ESI-TOF) m/z Calcd for C₂₁H₂₃O₄⁺ [M+H]⁺ 339.1596, found 339.1590.

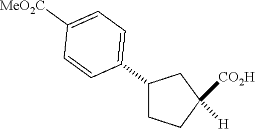 [SMILES: COC(=O)c1ccc([C@H]2CC[C@H](C(=O)O)C2)cc1]

### cis-3-(4-(methoxycarbonyl)phenyl)cyclopentane-1-carboxylic acid (3s)

Following General Procedure 1 on 0.1 mmol scale. The product was obtained as colorless oil (17.8 mg, 72% yield). ¹H NMR (600 MHz, CDCl₃) δ 7.99-7.94 (m, 2H), 7.35-7.31 (m, 2H), 3.90 (s, 3H), 3.14 (tt, J=11.0, 7.0 Hz, 1H), 3.05-2.96 (m, 1H), 2.46-2.39 (m, 1H), 2.21-2.10 (m, 2H), 2.11-2.03 (m, 1H), 1.98 (ddd, J=12.8, 11.4, 9.6 Hz, 1H), 1.86-1.76 (m, 1H); ¹³C NMR (151 MHz, CDCl₃) δ 182.3, 167.2, 149.9, 129.9, 128.3, 127.3, 52.2, 46.3, 43.6, 38.0, 33.9, 29.4; HRMS (ESI-TOF) m/z Calcd for C₁₄H₁₇₀₄⁺[M+H]⁺ 249.1127, found 249.1137.

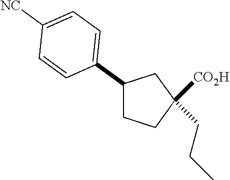 [SMILES: CCC[C@]1(C(=O)O)CC[C@@H](c2ccc(C#N)cc2)C1]

### cis-3-(4-cyanophenyl)-1-propylcyclopentane-1-carboxylic acid (4a)

Following General Procedure 1 on 0.1 mmol scale. The product was obtained as colorless oil (20.3 mg, 79% yield). ¹H NMR (600 MHz, CDCl₃) δ 7.57 (d, J=8.1 Hz, 2H), 7.35 (d, J=8.0 Hz, 2H), 3.18 (ddd, J=18.3, 10.6, 7.4 Hz, 1H), 2.47-2.36 (m, 1H), 2.23 (dd, J=13.5, 10.6 Hz, 1H), 2.12 (dd, J=13.2, 7.8 Hz, 2H), 1.81-1.61 (m, 4H), 1.40-1.27 (m, 2H), 0.94 (t, J=7.2 Hz, 3H); ¹³C NMR (151 MHz, CDCl₃) δ 183.8, 150.5, 132.4, 128.1, 119.2, 110.1, 53.7, 45.2, 43.2, 42.0, 36.7, 34.4, 19.1, 14.6; HRMS (ESI-TOF) m/z Calcd for C₁₆H₂₀NO₂⁺[M+H]⁺ 258.1494, found 258.1503.

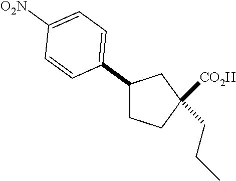 [SMILES: CCC[C@]1(C(=O)O)CC[C@@H](c2ccc([N+](=O)[O-])cc2)C1]

### cis-3-(4-nitrophenyl)-1-propylcyclopentane-1-carboxylic acid (4b)

Following General Procedure 1 on 0.1 mmol scale. The product was obtained as colorless oil (19.9 mg, 72% yield). ¹H NMR (600 MHz, CDCl₃) δ 7.57 (d, J=8.1 Hz, 2H), 7.35 (d, J=8.0 Hz, 2H), 3.18 (ddd, J=18.3, 10.6, 7.4 Hz, 1H), 2.47-2.36 (m, 1H), 2.23 (dd, J=13.5, 10.6 Hz, 1H), 2.17-2.12 (m, 2H), 1.81-1.61 (m, 4H), 1.40-1.27 (m, 2H), 0.94 (t, J=7.2 Hz, 3H); ¹³C NMR (151 MHz, CDCl₃) δ 184.0, 152.7, 146.6, 128.1, 123.8, 53.8, 45.0, 43.2, 42.0, 36.7, 34.5, 19.1, 14.6; HRMS (ESI-TOF) m/z Calcd for C₁₅H₁₈NO₄⁻ [M−H]⁻ 276.1236, found 276.1236.

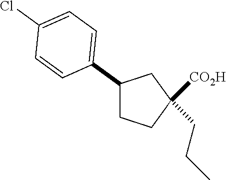 [SMILES: CCC[C@]1(C(=O)O)CC[C@@H](c2ccc(Cl)cc2)C1]

### cis-3-(4-chlorophenyl)-1-propylcyclopentane-1-carboxylic acid (4c)

Following General Procedure 1 on 0.1 mmol scale. The product was obtained as colorless oil (18.4 mg, 69% yield). ¹H NMR (600 MHz, CDCl₃) δ 7.26-7.24 (m, 2H), 7.20-7.17 (m, 2H), 3.10 (tt, J=11.0, 7.8 Hz, 1H), 2.41 (ddd, J=13.1, 7.6, 2.4 Hz, 1H), 2.19 (dd, J=13.5, 10.8 Hz, 1H), 2.13-2.04 (m, 2H), 1.80-1.61 (m, 4H), 1.37-1.30 (m, 2H), 0.94 (t, J=7.3 Hz, 3H); ¹³C NMR (151 MHz, CDCl₃) δ 184.4, 143.2, 131.9, 128.6, 128.6, 53.5, 44.5, 43.6, 42.1, 36.5, 34.6, 19.1, 14.7; HRMS (ESI-TOF) m/z Calcd for C₁₅H₁₈ClO₂⁻ [M−H]⁻ 265.0995, found 265.0998.

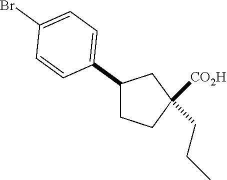 [SMILES: CCC[C@]1(C(=O)O)CC[C@@H](c2ccc(Br)cc2)C1]

### cis-3-(4-bromophenyl)-1-propylcyclopentane-1-carboxylic acid (4d)

Following General Procedure 1 on 0.1 mmol scale. The product was obtained as colorless oil (19.6 mg, 63% yield). ¹H NMR (600 MHz, CDCl₃) δ 7.43-7.38 (m, 2H), 7.15-7.11 (m, 2H), 3.09 (tt, J=11.0, 7.6 Hz, 1H), 2.41 (ddd, J=13.0, 7.6, 2.4 Hz, 1H), 2.19 (dd, J=13.5, 10.8 Hz, 1H), 2.14-2.04 (m, 2H), 1.79-1.60 (m, 4H), 1.37-1.29 (m, 2H), 0.94 (t, J=7.3 Hz, 3H); ¹³C NMR (151 MHz, CDCl₃) δ 184.3, 143.7, 131.5, 129.0, 119.9, 53.5, 44.6, 43.5, 42.1, 36.5, 34.6, 19.1, 14.7; HRMS (ESI-TOF) m/z Calcd for C₁₅H₁₈BrO₂⁺[M−H]⁻ 309.0490, found 309.0485.

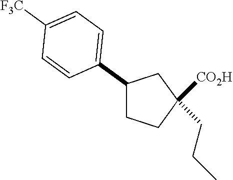 [SMILES: CCC[C@]1(C(=O)O)CC[C@@H](c2ccc(C(F)(F)F)cc2)C1]

### cis-1-propyl-3-(4-(trifluoromethyl)phenyl)cyclopentane-1-carboxylic acid (4e)

Following General Procedure 1 on 0.1 mmol scale. The product was obtained as colorless oil (25.5 mg, 85% yield). ¹H NMR (600 MHz, CDCl₃) δ 7.56-7.50 (m, 2H), 7.39-7.33 (m, 2H), 3.19 (tt, J=11.0, 7.4 Hz, 1H), 2.43 (ddd, J=13.2, 7.6, 2.4 Hz, 1H), 2.25 (dd, J=13.6, 10.8 Hz, 1H), 2.17-2.07 (m, 2H), 1.85-1.63 (m, 4H), 1.40-1.29 (m, 2H), 0.95 (t, J=7.3 Hz, 3H); ¹³C NMR (151 MHz, CDCl₃) δ 183.8, 148.87 (q, J=1.6 Hz), 128.59 (q, J=32.4 Hz) 127.6, 124.44 (q, J=271.7 Hz), 125.41 (q, J=3.7 Hz), 53.6, 44.9, 43.4, 42.1, 36.6, 34.5, 19.1, 14.7; HRMS (ESI-TOF) m/z Calcd for C₁₆H₁₈F₃O₂⁻ [M−H]⁻ 299.1259, found 299.1255.

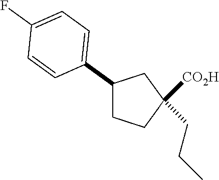 [SMILES: CCC[C@]1(C(=O)O)CC[C@@H](c2ccc(F)cc2)C1]

### cis-3-(4-fluorophenyl)-1-propylcyclopentane-1-carboxylic acid (4f)

Following General Procedure 1 on 0.1 mmol scale. The product was obtained as colorless oil (15.7 mg, 63% yield). ¹H NMR (600 MHz, CDCl₃) δ 7.23-7.18 (m, 2H), 7.00-6.94 (m, 2H), 3.11 (tt, J=11.0, 7.5 Hz, 1H), 2.41 (ddd, J=13.1, 7.6, 2.4 Hz, 1H), 2.18 (dd, J=13.5, 10.9 Hz, 1H), 2.15-2.04 (m, 2H), 1.79-1.60 (m, 4H), 1.37-1.31 (m, 2H), 0.94 (t, J=7.3 Hz, 3H); ¹³C NMR (151 MHz, CDCl₃) δ 183.9, 161.48 (d, J=243.8 Hz), 140.30 (d, J=3.3 Hz), 128.56 (d, J=7.8 Hz), 115.18 (d, J=21.2 Hz). 53.5, 44.4, 43.9, 42.2, 36.5, 34.8, 19.1, 14.7; HRMS (ESI-TOF) m/z Calcd for C₁₅H₁₈FO₂⁻ [M−H]⁻ 249.1291, found 249.1297.

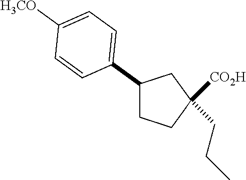 [SMILES: CCC[C@]1(C(=O)O)CC[C@@H](c2ccc(OC)cc2)C1]

### cis-3-(4-methoxyphenyl)-1-propylcyclopentane-1-carboxylic acid (4g)

Following General Procedure 1 on 0.1 mmol scale. The product was obtained as colorless oil (14.4 mg, 55% yield). ¹H NMR (600 MHz, CDCl₃) δ 7.20-7.16 (m, 2H), 6.86-6.82 (m, 2H), 3.79 (s, 3H), 3.08 (tt, J=11.1, 7.3 Hz, 1H), 2.40 (ddd, J=13.1, 7.7, 2.3 Hz, 1H), 2.18 (dd, J=13.4, 11.0 Hz, 1H), 2.13-2.03 (m, 2H), 1.79-1.60 (m, 4H), 1.40-1.30 (m, 2H), 0.94 (t, J=7.3 Hz, 3H); ¹³C NMR (151 MHz, CDCl₃) δ 184.3, 158.1, 136.7, 128.1, 113.9, 55.4, 53.4, 44.4, 44.0, 42.2, 36.4, 34.8, 19.1, 14.7; HRMS (ESI-TOF) m/z Calcd for C₁₆H₂₃O₃⁺[M+H]⁺ 261.1491, 261.1501.

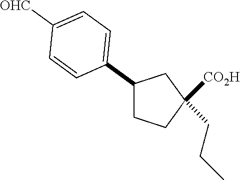 [SMILES: CCC[C@]1(C(=O)O)CC[C@@H](c2ccc(C=O)cc2)C1]

### cis-3-(4-formylphenyl)-1-propylcyclopentane-1-carboxylic acid (4h)

Following General Procedure 1 on 0.1 mmol scale. The product was obtained as colorless oil (20.5 mg, 79% yield). ¹H NMR (600 MHz, CDCl₃) δ 9.97 (s, 1H), 7.84-7.78 (m, 2H), 7.42 (d, J=8.0 Hz, 2H), 3.22 (tt, J=10.9, 7.4 Hz, 1H), 2.44 (ddd, J=13.2, 7.7, 2.4 Hz, 1H), 2.27 (dd, J=13.6, 10.7 Hz, 1H), 2.19-2.10 (m, 2H), 1.87-1.65 (m, 4H), 1.38-1.31 (m, 2H), 0.95 (t, J=7.3 Hz, 3H); ¹³C NMR (151 MHz, CDCl₃) δ 192.1, 183.4, 152.2, 134.9, 130.1, 128.0, 53.6, 45.3, 43.3, 42.1, 36.6, 34.5, 19.1, 14.7; HRMS (ESI-TOF) m/z Calcd for C₁₆H₂₁O₃⁺ [M+H]⁺ 261.1491, found 261.1496.

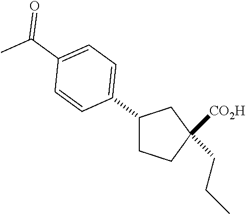 [SMILES: CCC[C@]1(C(=O)O)CC[C@H](c2ccc(C(C)=O)cc2)C1]

### cis-3-(4-acetylphenyl)-1-propylcyclopentane-1-carboxylic acid (4i)

Following General Procedure 1 on 0.1 mmol scale. The product was obtained as colorless oil (21.6 mg, 79% yield). ¹H NMR (600 MHz, CDCl₃) δ 7.91-7.86 (m, 2H), 7.37-7.32 (m, 2H), 3.19 (tt, J=10.9, 7.4 Hz, 1H), 2.58 (s, 3H), 2.43 (ddd, J=13.2, 7.7, 2.5 Hz, 1H), 2.25 (dd, J=13.6, 10.8 Hz, 1H), 2.16-2.08 (m, 2H), 1.83-1.63 (m, 4H), 1.39-1.29 (m, 2H), 0.94 (t, J=7.3 Hz, 3H); ¹³C NMR (151 MHz, CDCl₃) δ 198.2, 184.2, 150.7, 135.4, 128.7, 127.5, 53.7, 45.1, 43.4, 42.1, 36.5, 34.4, 26.7, 19.1, 14.6.

HRMS (ESI-TOF) m/z C₁₇H₂₁O₃⁻ [M−H]⁻ 273.1491, found 273.1500.

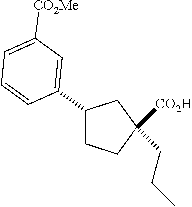 [SMILES: CCC[C@]1(C(=O)O)CC[C@H](c2cccc(C(=O)OC)c2)C1]

### cis-3-(3-(methoxycarbonyl)phenyl)-1-propylcyclopentane-1-carboxylic acid (4j)

Following General Procedure 1 on 0.1 mmol scale. The product was obtained as colorless oil (21.5 mg, 74% yield). ¹H NMR (600 MHz, CDCl₃) δ 7.93 (tt, J=1.9, 0.6 Hz, 1H), 7.86 (ddd, J=7.7, 1.7, 1.2 Hz, 1H), 7.47-7.45 (m, 1H), 7.36 (td, J=7.7, 0.5 Hz, 1H), 3.91 (s, 3H), 3.19 (tt, J=11.1, 7.3 Hz, 1H), 2.44 (ddd, J=13.1, 7.9, 2.4 Hz, 1H), 2.24 (dd, J=13.5, 11.0 Hz, 1H), 2.17-2.08 (m, 2H), 1.84-1.62 (m, 4H), 1.38-1.31 (m, 2H), 0.94 (t, J=7.3 Hz, 3H); ¹³C NMR (151 MHz, CDCl₃) δ 184.1, 167.4, 145.0, 131.9, 130.3, 128.6, 128.5, 127.6, 53.5, 52.2, 44.9, 43.6, 42.1, 36.4, 34.4, 19.1, 14.7; HRMS (ESI-TOF) m/z Calcd for C₁₇H₂₂NaO₄⁺[M+Na]+313.1416, found 313.1422.

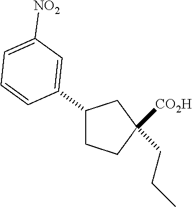 [SMILES: CCC[C@]1(C(=O)O)CC[C@H](c2cccc([N+](=O)[O-])c2)C1]

### cis-3-(3-nitrophenyl)-1-propylcyclopentane-1-carboxylic acid (4k)

Following General Procedure 1 on 0.1 mmol scale. The product was obtained as colorless oil (23.2 mg, 84% yield). ¹H NMR (600 MHz, CDCl₃) δ 8.14-8.10 (m, 1H), 8.05 (ddd, J=8.1, 2.3, 1.1 Hz, 1H), 7.60 (ddd, J=7.7, 1.7, 0.8 Hz, 1H), 7.45 (t, J=7.9 Hz, 1H), 3.30-3.18 (m, 1H), 2.46 (ddd, J=13.1, 7.6, 2.4 Hz, 1H), 2.27 (dd, J=13.6, 10.6 Hz, 1H), 2.20-2.13 (m, 2H), 1.86-1.66 (m, 4H), 1.39-1.32 (m, 2H), 0.95 (t, J=7.3 Hz, 3H). ¹³C NMR (151 MHz, CDCl₃) δ 183.9, 148.5, 146.9, 133.6, 129.4, 122.3, 121.4, 53.7, 44.7, 43.3, 42.1, 36.6, 34.4, 19.1, 14.6; HRMS (ESI-TOF) m/z Calcd for C₁₅H₁₈NO₄⁻ [M−H]⁻ 276.1236, found 276.1235.

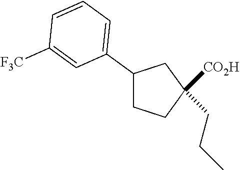 [SMILES: CCC[C@]1(C(=O)O)CCC(c2cccc(C(F)(F)F)c2)C1]

### cis-1-propyl-3-(3-(trifluoromethyl)phenyl)cyclopentane-1-carboxylic acid (41)

Following General Procedure 1 on 0.1 mmol scale. The product was obtained as colorless oil (21.9 mg, 73% yield). ¹H NMR (600 MHz, CDCl₃) δ 7.51-7.48 (m, 1H), 7.46-7.43 (m, 2H), 7.41-7.39 (m, 2H), 3.19 (tt, J=11.0, 7.6 Hz, 1H), 2.44 (ddd, J=13.3, 7.8, 2.4 Hz, 1H), 2.24 (dd, J=13.6, 10.8 Hz, 1H), 2.18-2.10 (m, 2H), 1.83-1.64 (m, 4H), 1.41-1.30 (m, 2H), 0.95 (t, J=7.3 Hz, 3H); ¹³C NMR (151 MHz, CDCl₃) δ 184.1, 145.7, 130.74 (q, J=31.9 Hz), 130.60 (q, J=1.3 Hz), 128.9, 124.39 (q, J=272.3 Hz), 124.07 (q, J=3.8 Hz), 123.12 (q, J=3.9 Hz), 53.6, 44.9, 43.5, 42.1, 36.5, 34.5, 19.1, 14.6; HRMS (ESI-TOF) m/z Calcd for C₁₆H₁₈F₃O₂⁻ [M−H]⁻ 299.1259, found 299.1255.

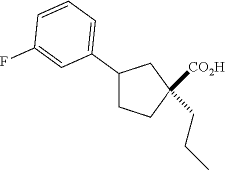 [SMILES: CCC[C@]1(C(=O)O)CCC(c2cccc(F)c2)C1]

### cis-3-(3-fluorophenyl)-1-propylcyclopentane-1-carboxylic acid (4m)

Following General Procedure 1 on 0.1 mmol scale. The product was obtained as colorless oil (16.8 mg, 67% yield). ¹H NMR (600 MHz, CDCl₃) δ 7.24 (td, J=8.0, 6.1 Hz, 1H), 7.03 (ddt, J=7.6, 1.5, 0.7 Hz, 1H), 6.98-6.93 (m, 1H), 6.87 (tdd, J=8.5, 2.6, 1.0 Hz, 1H), 3.13 (tt, J=11.0, 7.3 Hz, 1H), 2.42 (ddd, J=13.1, 7.8, 2.4 Hz, 1H), 2.21 (dd, J=13.5, 10.9 Hz, 1H), 2.15-2.06 (m, 2H), 1.81-1.61 (m, 4H), 1.39-1.28 (m, 2H), 0.94 (t, J=7.3 Hz, 3H); ¹³C NMR (151 MHz, CDCl₃) δ 184.1, 163.10 (d, J=245.3 Hz), 147.43 (d, J=1.6 Hz), 129.84 (d, J=8.3 Hz), 122.90 (d, J=2.8 Hz), 114.04 (d, J=21.4 Hz), 112.99 (d, J=21.5 Hz), 53.5, 44.9, 44.8, 43.5, 42.1, 36.4, 34.5, 19.1, 14.7; HRMS (ESI-TOF) m/z Calcd for C₁₅H₁₈FO₂⁻ [M−H]⁻ 249.1291, found 249.1297.

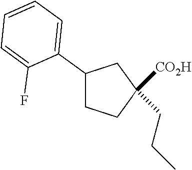 [SMILES: CCC[C@]1(C(=O)O)CCC(c2ccccc2F)C1]

### cis-3-(2-fluorophenyl)-1-propylcyclopentane-1-carboxylic acid (4n)

Following General Procedure 1 on 0.1 mmol scale. The product was obtained as colorless oil (20.8 mg, 83% yield). ¹H NMR (600 MHz, CDCl₃) δ 7.30 (td, J=7.6, 1.7 Hz, 1H), 7.18-7.14 (m, 1H), 7.08 (td, J=7.5, 1.3 Hz, 1H), 7.00 (ddd, J=10.6, 8.1, 1.3 Hz, 1H), 3.41 (tt, J=11.0, 7.5 Hz, 1H), 2.42 (ddd, J=13.1, 7.8, 2.7 Hz, 1H), 2.22 (dd, J=13.3, 10.9 Hz, 1H), 2.16-2.01 (m, 2H), 1.85-1.56 (m, 4H), 1.43-1.30 (m, 2H), 0.95 (t, J=7.3 Hz, 3H); ¹³C NMR (151 MHz, CDCl₃) δ 184.3, 161.2 (d, J=244.4 Hz), 131.28 (d, J=14.8 Hz), 127.94 (d, J=4.9 Hz), 127.62 (d, J=8.2 Hz), 124.21 (d, J=3.3 Hz), 115.31 (d, J=22.5 Hz), 53.4, 42.3, 42.0, 37.7, 37.7, 36.2, 33.0, 19.1, 14.7; HRMS (ESI-TOF) m/z Calcd for C₁₅H₁₈FO₂⁻ [M−H]⁻ 249.1291, found 249.1294

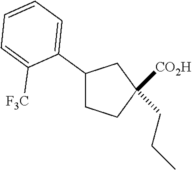 [SMILES: CCC[C@]1(C(=O)O)CCC(c2ccccc2C(F)(F)F)C1]

### cis-1-propyl-3-(2-(trifluoromethyl)phenyl)cyclopentane-1-carboxylic acid (4o)

Following General Procedure 1 on 0.1 mmol scale. The product was obtained as colorless oil (24.3 mg, 81% yield). ¹H NMR (600 MHz, CDCl₃) δ 7.62-7.57 (m, 2H), 7.51 (ddd, J=8.6, 7.4, 1.4 Hz, 1H), 7.30-7.25 (m, 1H), 3.52 (ddd, J=18.2, 10.8, 7.6 Hz, 1H), 2.45 (ddd, J=13.2, 7.5, 2.5 Hz, 1H), 2.25 (dd, J=13.6, 10.6 Hz, 1H), 2.17-2.05 (m, 2H), 1.89-1.79 (m, 1H), 1.77-1.68 (m, 3H), 1.39-1.32 (m, 2H), 0.96 (t, J=7.3 Hz, 3H); ¹³C NMR (151 MHz, CDCl₃) δ 184.4, 144.1, 132.2, 128.58 (q, J=29.1 Hz), 128.2, δ 124.80 (q, J=273.6 Hz), 126.1, 125.63 (q, J=6.0 Hz), 53.8, 44.7, 41.8, 40.5, 40.4, 40.4, 40.4, 36.9, 35.6, 19.1, 14.7; HRMS (ESI-TOF) m/z Calcd for C₁₆H₁₈F₃O₂⁻ [M−H]⁻ 299.1259, found 299.1263.

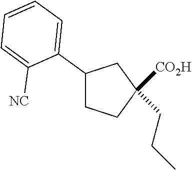 [SMILES: CCC[C@]1(C(=O)O)CCC(c2ccccc2C#N)C1]

### cis-3-(2-cyanophenyl)-1-propylcyclopentane-1-carboxylic acid (4p)

Following General Procedure 1 on 0.1 mmol scale. The product was obtained as colorless oil (20.5 mg, 80% yield). ¹H NMR (600 MHz, CDCl₃) δ 7.60 (ddd, J=7.7, 1.5, 0.5 Hz, 1H), 7.54 (td, J=7.7, 1.4 Hz, 1H), 7.51-7.47 (m, 1H), 7.28 (td, J=7.5, 1.3 Hz, 1H), 3.58 (tdd, J=10.6, 8.2, 6.6 Hz, 1H), 2.50-2.40 (m, 1H), 2.31-2.13 (m, 3H), 1.85-1.67 (m, 4H), 1.43-1.30 (m, 2H), 0.95 (t, J=7.3 Hz, 3H); ¹³C NMR (151 MHz, CDCl₃) δ 184.2, 148.6, 133.3, 132.9, 126.8, 126.8, 118.4, 112.5, 53.8, 43.2, 42.8, 41.9, 36.7, 34.2, 19.1, 14.6; HRMS (ESI-TOF) m/z Calcd for C₁₆H₁₉NNaO₂⁺[M+Na]⁺280.1313, found 280.1313.

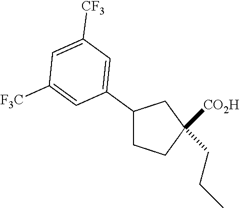 [SMILES: CCC[C@]1(C(=O)O)CCC(c2cc(C(F)(F)F)cc(C(F)(F)F)c2)C1]

### cis-3-(3,5-bis(trifluoromethyl)phenyl)-1-propylcyclopentane-1-carboxylic acid (4q)

Following General Procedure 1 on 0.1 mmol scale. The product was obtained as colorless oil (27.6 mg, 75% yield). ¹H NMR (600 MHz, CDCl₃) δ 7.70 (d, J=3.5 Hz, 3H), 3.27 (tt, J=10.8, 7.5 Hz, 1H), 2.46 (ddd, J=13.2, 7.5, 2.3 Hz, 1H), 2.26 (dd, J=13.6, 10.5 Hz, 1H), 2.21-2.13 (m, 2H), 1.84-1.66 (m, 4H), 1.39-1.32 (m, 2H), 0.95 (t, J=7.3 Hz, 3H); ¹³C NMR (151 MHz, CDCl₃) δ 183.6, 147.3, 131.71 (q, J=33.0 Hz), 127.52 (q, J=3.7 Hz), 123.58 (q, J=272.7 Hz), 120.35 (p, J=3.9 Hz), 53.7, 44.7, 43.1, 42.1, 36.6, 34.4, 19.1, 14.6; HRMS (ESI-TOF) m/z Calcd for C₁₇H₁₇F₆O₂⁺ [M−H]⁻ 367.1133, found 367.1128.

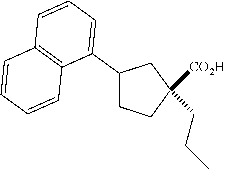 [SMILES: CCC[C@]1(C(=O)O)CCC(c2cccc3ccccc23)C1]

### cis-3-(naphthalen-1-yl)-1-propylcyclopentane-1-carboxylic acid (4r)

Following General Procedure 1 on 0.1 mmol scale. The product was obtained as colorless oil (16.9 mg, 60% yield). ¹H NMR (600 MHz, CDCl₃) δ 8.14-8.10 (m, 1H), 7.89-7.85 (m, 1H), 7.74-7.70 (m, 1H), 7.55-7.47 (m, 3H), 7.44 (dd, J=8.1, 7.3 Hz, 1H), 3.92 (tt, J=10.8, 7.1 Hz, 1H), 2.51 (ddd, J=13.2, 7.8, 2.8 Hz, 1H), 2.39-2.21 (m, 3H), 2.02-1.95 (m, 1H), 1.92-1.75 (m, 3H), 1.47-1.37 (m, 2H), 1.00 (t, J=7.3 Hz, 3H); ¹³C NMR (151 MHz, CDCl₃) δ 184.5, 140.2, 134.0, 132.3, 129.0, 126.8, 125.9, 125.8, 125.5, 123.6, 122.4, 53.3, 43.2, 42.3, 40.3, 36.0, 33.5, 19.1, 14.7; HRMS (ESI-TOF) m/z Calcd for C₁₉H₂₁O₂—[M−H]⁻ 281.1542, found 281.1552.

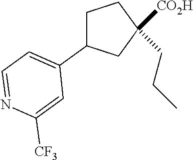 [SMILES: CCC[C@@]1(C(=O)O)CCC(c2ccnc(C(F)(F)F)c2)C1]

### cis-1-propyl-3-(2-(trifluoromethyl)pyridin-4-yl)cyclopentane-1-carboxylic acid (4s)

Following General Procedure 1 on 0.1 mmol scale. The product was obtained as colorless oil (24.6 mg, 82% yield). ¹H NMR (600 MHz, CDCl₃) δ 8.62 (dt, J=5.0, 0.6 Hz, 1H), 7.57 (dt, J=1.6, 0.7 Hz, 1H), 7.38 (ddt, J=4.5, 1.2, 0.6 Hz, 1H), 3.21 (ddd, J=18.0, 10.4, 7.6 Hz, 1H), 2.45 (ddd, J=13.1, 7.2, 2.5 Hz, 1H), 2.28 (dd, J=13.7, 10.3 Hz, 1H), 2.21-2.11 (m, 2H), 1.85-1.66 (m, 4H), 1.40-1.30 (m, 2H), 0.95 (t, J=7.3 Hz, 3H); ¹³C NMR (151 MHz, CDCl₃) δ 182.7, 156.1, 150.1, 148.43 (q, J=34.1 Hz), 125.3, 121.77 (q, J=274.3 Hz), 119.56 (q, J=2.8 Hz), 53.8, 44.4, 42.4, 42.0, 36.8, 33.9, 19.2, 14.6; HRMS (ESI-TOF) m/z Calcd for C₁₅H₁₉F₃NO₂⁺[M+H]302.1368, found 302.1368.

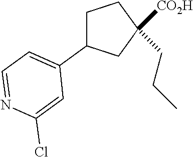 [SMILES: CCC[C@@]1(C(=O)O)CCC(c2ccnc(Cl)c2)C1]

### cis-3-(2-chloropyridin-4-yl)-1-propylcyclopentane-1-carboxylic acid (4t)

Following General Procedure 1 on 0.1 mmol scale. The product was obtained as colorless oil (18.7 mg, 70% yield). ¹H NMR (600 MHz, CDCl₃) δ 8.28 (dd, J=5.2, 0.6 Hz, 1H), 7.22 (dt, J=1.4, 0.7 Hz, 1H), 7.11 (ddd, J=5.2, 1.6, 0.6 Hz, 1H), 3.16-3.06 (m, 1H), 2.46-2.39 (m, 1H), 2.24 (dd, J=13.7, 10.4 Hz, 1H), 2.16-2.08 (m, 2H), 1.80-1.63 (m, 4H), 1.38-1.29 (m, 2H), 0.94 (t, J=7.3 Hz, 3H); ¹³C NMR (151 MHz, CDCl₃) δ 183.1, 157.5, 151.8, 149.6, 123.2, 121.6, 53.7, 44.1, 42.4, 42.0, 36.6, 33.8, 19.1, 14.6; HRMS (ESI-TOF) m/z Calcd for C₁₄H₁₉ClNO₂⁺ [M+H]⁺ 268.1104, found 268.1109.

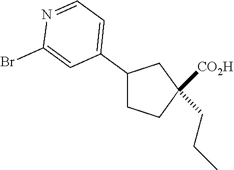 [SMILES: CCC[C@]1(C(=O)O)CCC(c2ccnc(Br)c2)C1]

### cis-3-(2-bromopyridin-4-yl)-1-propylcyclopentane-1-carboxylic acid (4u)

Following General Procedure 1 on 0.1 mmol scale. The product was obtained as colorless oil (19.2 mg, 62% yield). ¹H NMR (600 MHz, CDCl₃) δ 8.26 (dd, J=5.2, 0.7 Hz, 1H), 7.38 (dd, J=1.5, 0.8 Hz, 1H), 7.17-7.13 (m, 1H), 3.14-3.04 (m, 1H), 2.42 (ddd, J=10.6, 5.9, 1.4 Hz, 1H), 2.23 (dd, J=13.7, 10.4 Hz, 1H), 2.15-2.07 (m, 2H), 1.78-1.62 (m, 4H), 1.38-1.28 (m, 2H), 0.93 (t, J=7.3 Hz, 3H); ¹³C NMR (151 MHz, CDCl₃) δ 183.4, 157.4, 150.0, 142.4, 127.0, 122.0, 53.8, 44.1, 42.4, 42.0, 36.6, 33.8, 19.1, 14.6.; HRMS (ESI-TOF) m/z Calcd for C₁₄H₁₇BrNO₂⁻ [M−H]⁻ 310.0443, found 310.0446.

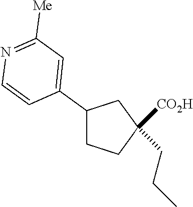 [SMILES: CCC[C@]1(C(=O)O)CCC(c2ccnc(C)c2)C1]

### cis-3-(2-methylpyridin-4-yl)-1-propylcyclopentane-1-carboxylic acid (4v)

Following General Procedure 1 on 0.1 mmol scale. The product was obtained as colorless oil (12.6 mg, 51% yield). ¹H NMR (600 MHz, CDCl₃) δ 8.40 (d, J=5.3 Hz, 1H), 7.07 (dd, J=10.7, 3.2 Hz, 2H), 3.12-3.06 (m, 1H), 2.53 (s, 3H), 2.44 (ddd, J=13.0, 7.5, 2.5 Hz, 1H), 2.28 (dd, J=13.6, 10.3 Hz, 1H), 2.13-2.05 (m, 2H), 1.79-1.68 (m, 3H), 1.64 (ddd, J=13.1, 10.9, 6.9 Hz, 1H), 1.38-1.32 (m, 2H), 0.94 (t, J=7.3 Hz, 3H); ¹³C NMR (151 MHz, CDCl₃) δ 181.9, 157.6, 155.8, 148.0, 122.8, 120.3, 53.9, 44.4, 42.8, 42.3, 36.7, 34.0, 23.6, 19.2, 14.7; HRMS (ESI-TOF) m/z Calcd for C₁₅H₂₂NO₂⁺[M+H]248.1651, found 248.1642.

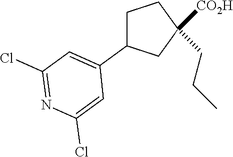 [SMILES: CCC[C@@]1(C(=O)O)CCC(c2cc(Cl)nc(Cl)c2)C1]

### cis-3-(2,6-dichloropyridin-4-yl)-1-propylcyclopentane-1-carboxylic acid (4w)

Following General Procedure 1 on 0.1 mmol scale. The product was obtained as colorless oil (26.3 mg, 87% yield). ¹H NMR (600 MHz, CDCl₃) δ 7.15 (s, 2H), 3.10 (ddd, J=18.0, 10.3, 7.5 Hz, 1H), 2.42 (ddd, J=13.0, 7.0, 2.9 Hz, 1H), 2.22 (dd, J=13.6, 10.3 Hz, 1H), 2.16-2.07 (m, 2H), 1.78-1.61 (m, 4H), 1.39-1.28 (m, 2H), 0.94 (t, J=7.3 Hz, 3H); ¹³C NMR (151 MHz, CDCl₃) δ 183.6, 160.0, 150.7, 121.9, 53.7, 44.0, 42.1, 41.9, 36.6, 33.7, 19.1, 14.6; HRMS (ESI-TOF) m/z Calcd for C₁₄H₁₈C₁₂N₀₂⁺[M+H]302.0715, found 302.0710.

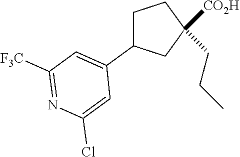 [SMILES: CCC[C@@]1(C(=O)O)CCC(c2cc(Cl)nc(C(F)(F)F)c2)C1]

### cis-3-(2-chloro-6-(trifluoromethyl)pyridin-4-yl)-1-propylcyclopentane-1-carboxylic acid (4x)

Following General Procedure 1 on 0.1 mmol scale. The product was obtained as colorless oil (20.4 mg, 61% yield). ¹H NMR (600 MHz, CDCl₃) δ 7.50 (d, J=1.3 Hz, 1H), 7.40 (dd, J=1.4, 0.7 Hz, 1H), 3.20 (tt, J=10.4, 8.5 Hz, 1H), 2.48-2.41 (m, 1H), 2.27 (dd, J=13.7, 10.1 Hz, 1H), 2.22-2.11 (m, 2H), 1.82-1.64 (m, 4H), 1.39-1.29 (m, 2H), 0.94 (t, J=7.3 Hz, 3H); ¹³C NMR (151 MHz, CDCl₃) δ 183.3, 159.3, 152.3, 148.42 (q, J=35.4 Hz), 126.1, 120.92 (q, J=274.6 Hz), 118.48 (q, J=2.8 Hz), 53.9, 44.2, 42.1, 41.9, 36.8, 33.8, 19.2, 14.6; HRMS (ESI-TOF) m/z Calcd for C₁₅H₁₈ClF₃NO₂⁺ [M+H]336.0978, found 336.0977.

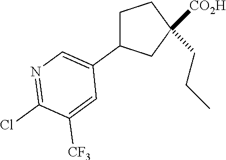 [SMILES: CCC[C@@]1(C(=O)O)CCC(c2cnc(Cl)c(C(F)(F)F)c2)C1]

### cis-3-(6-chloro-5-(trifluoromethyl)pyridin-3-yl)-1-propylcyclopentane-1-carboxylic acid (4y)

Following General Procedure 1 on 0.1 mmol scale. The product was obtained as colorless oil (21.1 mg, 63% yield). ¹H NMR (600 MHz, CDCl₃) δ 8.49-8.43 (m, 1H), 7.93 (dd, J=2.4, 0.7 Hz, 1H), 3.27-3.17 (m, 1H), 2.49-2.41 (m, 1H), 2.26 (dd, J=13.6, 10.0 Hz, 1H), 2.22-2.11 (m, 2H), 1.80-1.65 (m, 4H), 1.39-1.29 (m, 2H), 0.95 (t, J=7.3 Hz, 3H); ¹³C NMR (151 MHz, CDCl₃) δ 183.4, 151.43 (q, J=1.0 Hz), 146.61 (q, J=1.4 Hz), 139.6, 135.46 (q, J=4.8 Hz), 125.12 (q, J=33.0 Hz), 122.36 (q, J=272.9 Hz), 54.0, 42.6, 42.0, 41.6, 37.0, 34.5, 19.2, 14.6; HRMS (ESI-TOF) m/z Calcd for C₁₅H₁₈ClF₃NO₂⁺[M+H]⁺ 336.0978, found 336.0980.

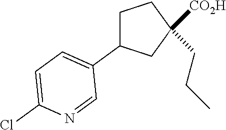 [SMILES: CCC[C@@]1(C(=O)O)CCC(c2ccc(Cl)nc2)C1]

### cis-3-(6-chloropyridin-3-yl)-1-propylcyclopentane-1-carboxylic acid (4z)

Following General Procedure 1 on 0.1 mmol scale. The product was obtained as colorless oil (19.0 mg, 71% yield). ¹H NMR (600 MHz, CDCl₃) δ 8.30-8.28 (m, 1H), 7.58 (ddd, J=8.2, 2.6, 0.5 Hz, 1H), 7.28-7.23 (m, 1H), 3.14 (tdd, J=10.7, 7.9, 6.7 Hz, 1H), 2.45-2.39 (m, 1H), 2.22 (dd, J=13.6, 10.4 Hz, 1H), 2.15-2.08 (m, 2H), 1.78-1.63 (m, 4H), 1.38-1.29 (m, 2H), 0.94 (t, J=7.3 Hz, 3H); ¹³C NMR (151 MHz, CDCl₃) δ 183.5, 149.2, 148.9, 139.2, 137.7, 124.2, 53.8, 43.0, 42.0, 41.8, 36.8, 34.5, 19.2, 14.6; HRMS (ESI-TOF) m/z Calcd for C₁₄H₁₉ClNO₂⁺[M+H]⁺ 268.1104, found 268.1104.

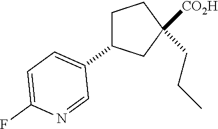 [SMILES: CCC[C@@]1(C(=O)O)CC[C@@H](c2ccc(F)nc2)C1]

### cis-3-(6-fluoropyridin-3-yl)-1-propylcyclopentane-1-carboxylic acid (4aa)

Following General Procedure 1 on 0.1 mmol scale. The product was obtained as colorless oil (13.8 mg, 55% yield). ¹H NMR (600 MHz, CDCl₃) δ 8.09 (ddt, J=2.5, 1.2, 0.7 Hz, 1H), 7.70 (dddd, J=8.3, 7.6, 2.6, 0.6 Hz, 1H), 6.87 (dd, J=8.5, 2.9 Hz, 1H), 3.15 (ddd, J=18.4, 10.7, 7.5 Hz, 1H), 2.47-2.38 (m, 1H), 2.21 (dd, J=13.7, 10.5 Hz, 1H), 2.16-2.08 (m, 1H), 1.81-1.59 (m, 5FH), 1.39-1.28 (m, 2H), 0.95 (t, J=7.3 Hz, 3H); ¹³C NMR (151 MHz, CDCl₃) δ 182.9, 162.54 (d, J=237.5 Hz), 146.28 (d, J=14.3 Hz), 139.81 (d, J=7.7 Hz), 137.67 (d, J=4.4 Hz), 109.34 (d, J=36.9 Hz), 53.7, 43.2, 42.1, 41.7, 36.8, 34.6, 19.2, 14.6; HRMS (ESI-TOF) m/z Calcd for C₁₄H₁₉FNO₂⁺[M+H]⁺ 252.1400, found 252.1412.

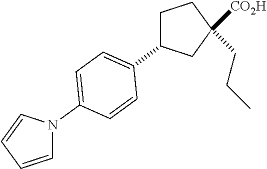 [SMILES: CCC[C@@]1(C(=O)O)CC[C@@H](c2ccc(-n3cccc3)cc2)C1]

### cis-3-(4-(1H-pyrrol-1-yl)phenyl)-1-propylcyclopentane-1-carboxylic acid (4ab)

Following General Procedure 1 on 0.1 mmol scale. The product was obtained as colorless oil (16.6 mg, 56% yield). ¹H NMR (600 MHz, CDCl₃) δ 7.31 (d, J=0.6 Hz, 4H), 7.08-7.04 (m, 2H), 6.34-6.32 (m, 2H), 3.16 (tdd, J=11.1, 8.0, 6.7 Hz, 1H), 2.44 (ddd, J=13.2, 7.8, 2.4 Hz, 1H), 2.24 (dd, J=13.5, 10.9 Hz, 1H), 2.17-2.07 (m, 2H), 1.84-1.63 (m, 4H), 1.39-1.33 (m, 2H), 0.95 (t, J=7.3 Hz, 3H); ¹³C NMR (151 MHz, CDCl₃) δ 184.0, 142.2, 139.1, 128.3, 120.7, 119.5, 110.3, 53.5, 44.6, 43.7, 42.1, 36.5, 34.7, 19.1, 14.7; HRMS (ESI-TOF) m/z Calcd for C₁₉H₂₄NO₂⁺[M+H]⁺ 298.1807, found 298.1797.

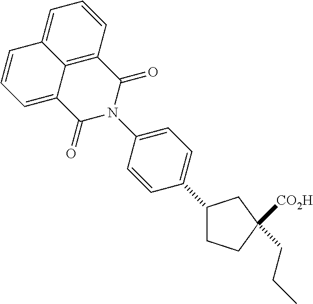

### cis-3-(4-(1,3-dioxo-1H-benzo[de]isoquinolin-2(3H)-yl)phenyl)-1-propylcyclopentane-1-carboxylic acid (4ac)

Following General Procedure 1 on 0.1 mmol scale. The product was obtained as colorless oil (29.9 mg, 70% yield). ¹H NMR (600 MHz, CDCl₃) δ 8.64 (dd, J=7.2, 1.1 Hz, 2H), 8.26 (dd, J=8.3, 1.1 Hz, 2H), 7.78 (dd, J=8.2, 7.2 Hz, 2H), 7.47-7.41 (m, 2H), 7.26-7.21 (m, 2H), 3.22 (tt, J=11.1, 7.3 Hz, 1H), 2.45 (ddd, J=13.1, 7.9, 2.4 Hz, 1H), 2.32 (dd, J=13.5, 11.0 Hz, 1H), 2.20-2.12 (m, 2H), 1.91-1.80 (m, 1H), 1.81-1.64 (m, 3H), 1.43-1.31 (m, 2H), 0.96 (t, J=7.3 Hz, 3H); ¹³C NMR (151 MHz, CDCl₃) δ 183.7, 164.6, 145.2, 134.3, 133.3, 131.9, 131.8, 128.7, 128.5, 128.3, 127.1, 123.0, 53.5, 44.9, 43.7, 42.2, 36.4, 34.6, 19.1, 14.7.; HRMS (ESI-TOF) m/z Calcd for C₂₇H₂₆NO₄⁺[M+H]428.1862, found 428.1862.

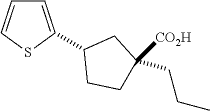

### cis-1-propyl-3-(thiophen-2-yl)cyclopentane-1-carboxylic acid (4ad)

Following General Procedure 1 on 0.1 mmol scale at 80° C. for 24 h. The product was obtained as colorless oil (10.5 mg, 44% yield). ¹H NMR (600 MHz CDCl₃) δ 7.12 (dd, J=5.1, 1.2 Hz, 1H), 6.91 (dd, J=5.1, 3.4 Hz, 1H), 6.83 (dt, J=3.4, 1.1 Hz, 1H), 3.38 (tt, J=10.5, 7.2 Hz, 1H), 2.41 (ddd, J=13.2, 7.9, 2.6 Hz, 1H), 2.27 (dd, J=13.4, 10.6 Hz, 1H), 2.22-2.14 (m, 2H), 1.87-1.60 (m, 4H), 1.38-1.28 (m, 2H), 0.93 (t, J=7.3 Hz, 3H); ¹³C NMR (151 MHz, CDCl₃) δ 182.1, 148.8, 126.8, 123.0, 122.9, 53.3, 44.5, 42.2, 40.3, 36.0, 35.5, 19.0, 14.6; HRMS (ESI-TOF) m/z Calcd for C₁₃H₁₉O₂S⁺ [M+H]⁺ 239.1106, found 239.1105.

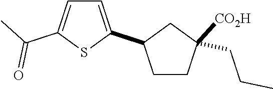 [SMILES: CCC[C@]1(C(=O)O)CC[C@@H](c2ccc(C(C)=O)s2)C1]

### cis-3-(5-acetylthiophen-2-yl)-1-propylcyclopentane-1-carboxylic acid (4ae)

Following General Procedure 1 on 0.1 mmol scale. The product was obtained as colorless oil (21.7 mg, 78% yield). ¹H NMR (600 MHz, CDCl₃) δ 7.53 (d, J=3.8 Hz, 1H), 6.87 (dd, J=3.8, 0.9 Hz, 1H), 3.38 (tt, J=10.5, 7.4 Hz, 1H), 2.51 (s, 3H), 2.41 (ddd, J=13.5, 7.8, 2.6 Hz, 1H), 2.28 (dd, J=13.6, 10.3 Hz, 1H), 2.22-2.16 (m, 2H), 1.80 (dtd, J=12.5, 10.7, 7.8 Hz, 1H), 1.74-1.61 (m, 3H), 1.37-1.28 (m, 2H), 0.93 (t, J=7.3 Hz, 3H); ¹³C NMR (151 MHz, CDCl₃) δ 190.8, 183.3, 158.9, 141.9, 133.0, 124.6, 53.6, 43.9, 42.0, 40.8, 36.2, 35.3, 26.6, 19.1, 14.6; HRMS (ESI-TOF) m/z Calcd for C₁₅H₁₉O₃S⁻ [M−H]⁻ 279.1055, found 279.1057.

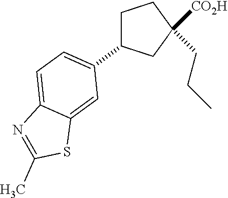 [SMILES: CCC[C@@]1(C(=O)O)CC[C@@H](c2ccc3nc(C)sc3c2)C1]

### cis-3-(2-methylbenzo[d]thiazol-6-yl)-1-propylcyclopentane-1-carboxylic acid (4af)

Following General Procedure 1 on 0.1 mmol scale. The product was obtained as colorless oil (19.1 mg, 63% yield). ¹H NMR (600 MHz, CDCl₃) δ 7.86 (dd, J=8.4, 0.5 Hz, 1H), 7.70 (dt, J=1.8, 0.6 Hz, 1H), 7.35 (ddd, J=8.4, 1.8, 0.5 Hz, 1H), 3.25 (tt, J=11.1, 7.6 Hz, 1H), 2.82 (s, 3H), 2.45 (ddd, J=13.1, 7.7, 2.4 Hz, 1H), 2.29 (dd, J=13.5, 10.9 Hz, 1H), 2.22-2.10 (m, 2H), 1.83 (dtd, J=12.4, 11.1, 7.7 Hz, 1H), 1.78-1.64 (m, 3H), 1.40-1.33 (m, 2H), 0.95 (t, J=7.3 Hz, 3H); ¹³C NMR (151 MHz, CDCl₃) δ 183.5, 166.6, 151.9, 141.8, 135.9, 125.8, 122.1, 119.5, 53.6, 45.1, 44.0, 42.2, 36.6, 35.0, 20.2, 19.1, 14.7; HRMS (ESI-TOF) m/z Calcd for C₁₇H₂₂NO₂S⁺ [M+H]⁺ 304.1371, found 304.1364.

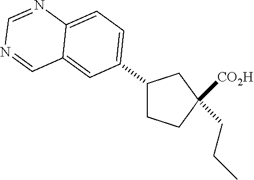 [SMILES: CCC[C@]1(C(=O)O)CC[C@H](c2ccc3ncncc3c2)C1]

### cis-1-propyl-3-(quinazolin-6-yl)cyclopentane-1-carboxylic acid (4ag)

Following General Procedure 1 on 0.1 mmol scale. The product was obtained as colorless oil (17.3 mg, 61% yield). ¹H NMR (600 MHz, CDCl₃) δ 9.39 (s, 1H), 9.28 (s, 1H), 8.01 (d, J=8.7 Hz, 1H), 7.90 (dd, J=8.8, 2.0 Hz, 1H), 7.82 (d, J=1.9 Hz, 1H), 3.38 (ddd, J=18.2, 10.4, 7.6 Hz, 1H), 2.50 (ddd, J=13.1, 7.5, 2.4 Hz, 1H), 2.41 (dd, J=13.6, 10.2 Hz, 1H), 2.26-2.18 (m, 2H), 1.88 (dtd, J=12.6, 11.0, 7.5 Hz, 1H), 1.76 (dddd, J=20.4, 13.2, 6.6, 4.3 Hz, 3H), 1.45-1.33 (m, 2H), 0.96 (t, J=7.3 Hz, 3H); ¹³C NMR (151 MHz, CDCl₃) δ 182.4, 160.0, 154.5, 149.1, 145.5, 135.1, 128.3, 125.2, 124.4, 53.9, 45.1, 43.2, 42.2, 36.9, 34.7, 19.3, 14.7; HRMS (ESI-TOF) m/z Calcd for C₁₇H₂₁N₂O₂⁺[M+H]⁺ 285.1603, found 285.1606.

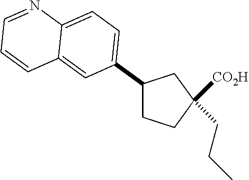 [SMILES: CCC[C@]1(C(=O)O)CC[C@@H](c2ccc3ncccc3c2)C1]

### cis-1-propyl-3-(quinolin-6-yl)cyclopentane-1-carboxylic acid (4ah)

Following General Procedure 1 on 0.1 mmol scale. The product was obtained as colorless oil (15.6 mg, 55% yield). ¹H NMR (600 MHz, CDCl₃) δ 8.90-8.82 (m, 1H), 8.17-8.05 (m, 2H), 7.76-7.70 (m, 1H), 7.67 (s, 1H), 7.45-7.37 (m, 1H), 3.36 (p, J=9.6 Hz, 1H), 2.54-2.46 (m, 1H), 2.46-2.38 (m, 1H), 2.24-2.15 (m, 2H), 1.96-1.86 (m, 1H), 1.84-1.66 (m, 3H), 1.45-1.34 (m, 2H), 0.97 (t, J=7.3 Hz, 3H).; ¹³C NMR (151 MHz, CDCl₃) δ 182.3, 149.2, 146.6, 143.9, 136.6, 130.2, 128.7, 128.5, 125.1, 121.3, 54.0, 45.1, 43.8, 42.4, 36.8, 34.8, 19.3, 14.7.; HRMS (ESI-TOF) m/z Calcd for C₁₈H₂₂NO₂⁺ [M+H]⁺ 284.1651, found 284.1658.

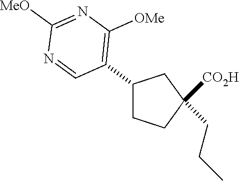 [SMILES: CCC[C@]1(C(=O)O)CC[C@H](c2cnc(OC)nc2OC)C1]

### cis-3-(2,4-dimethoxypyrimidin-5-yl)-1-propylcyclopentane-1-carboxylic acid (4ai)

Following General Procedure 1 on 0.1 mmol scale. The product was obtained as colorless oil (11.2 mg, 38% yield). ¹H NMR (600 MHz, CDCl₃) δ 8.15 (s, 1H), 3.98 (s, 3H), 3.96 (s, 3H), 3.22-3.12 (m, 1H), 2.39 (ddd, J=13.2, 7.5, 2.6 Hz, 1H), 2.19 (dd, J=13.4, 10.1 Hz, 1H), 2.08-1.98 (m, 2H), 1.79-1.65 (m, 3H), 1.60 (ddd, J=13.1, 10.8, 6.9 Hz, 1H), 1.39-1.28 (m, 2H), 0.93 (t, J=7.3 Hz, 3H); ¹³C NMR (151 MHz, CDCl₃) δ 182.9, 169.4, 163.9, 155.1, 117.7, 54.8, 54.0, 53.6, 42.2, 41.2, 36.2, 36.2, 32.0, 19.2, 14.7; HRMS (ESI-TOF) m/z Calcd for C₁₅H₂₃N₂O₄⁺[M+H]⁺ 295.1658, found 295.1657.

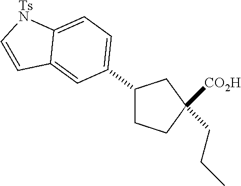 [SMILES: CCC[C@]1(C(=O)O)CC[C@H](c2ccc3c(ccn3S(=O)(=O)c3ccc(C)cc3)c2)C1]

### cis-1-propyl-3-(1-tosyl-1H-indol-5-yl)cyclopentane-1-carboxylic acid (4aj)

Following General Procedure 1 on 0.1 mmol scale. The product was obtained as colorless oil (27.6 mg, 65% yield). ¹H NMR (600 MHz, CDCl₃) δ 7.89 (d, J=8.6 Hz, 1H), 7.77-7.72 (m, 2H), 7.52 (d, J=3.6 Hz, 1H), 7.40 (d, J=1.7 Hz, 1H), 7.21 (ddd, J=8.7, 4.1, 1.3 Hz, 3H), 6.59 (dd, J=3.7, 0.8 Hz, 1H), 3.18 (tt, J=11.1, 7.3 Hz, 1H), 2.41 (ddd, J=13.2, 7.7, 2.3 Hz, 1H), 2.33 (s, 3H), 2.22 (dd, J=13.5, 11.0 Hz, 1H), 2.13-2.07 (m, 2H), 1.83-1.55 (m, 4H), 1.38-1.31 (m, 2H), 0.94 (t, J=7.3 Hz, 3H); ¹³C NMR (151 MHz, CDCl₃) δ 184.3, 145.0, 139.8, 135.5, 133.6, 131.0, 130.0, 127.0, 126.6, 124.3, 119.4, 113.5, 109.1, 53.5, 45.0, 44.1, 42.1, 36.5, 34.9, 21.7, 19.1, 14.7; HRMS (ESI-TOF) m/z Calcd for C₂₄H₂₈NO₄S⁺[M+H]⁺ 426.1739, found 426.1743.

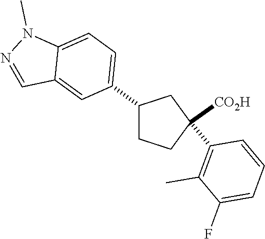 [SMILES: Cc1c(F)cccc1[C@]1(C(=O)O)CC[C@H](c2ccc3c(cnn3C)c2)C1]

### cis-1-(3-fluoro-2-methylphenyl)-3-(1-methyl-1H-indazol-5-yl)cyclopentane-1-carboxylic acid (4ak)

Following General Procedure 1 on 0.1 mmol scale. The product was obtained as colorless oil (21.8 mg, 62% yield). ¹H NMR (600 MHz, CDCl₃) δ 7.93 (d, J=0.9 Hz, 1H), 7.66 (dd, J=8.4, 0.8 Hz, 1H), 7.30 (s, 1H), 7.29-7.25 (m, 1H), 7.26-7.19 (m, 1H), 7.16 (dd, J=8.3, 1.4 Hz, 1H), 7.04-6.98 (m, 1H), 4.05 (s, 3H), 3.34 (tt, J=10.8, 7.6 Hz, 1H), 2.85-2.76 (m, 2H), 2.62 (dd, J=13.4, 7.8 Hz, 1H), 2.46-2.38 (m, 1H), 2.32-2.24 (m, 1H), 2.21 (d, J=2.6 Hz, 3H), 2.10-2.00 (m, 1H). ¹³C NMR (151 MHz, CDCl₃) δ 181.5, 162.19 (d, J=243.5 Hz), 144.21 (d, J=3.0 Hz), 143.2, 140.4, 132.6, 126.76 (d, J=9.3 Hz), 124.05 (d, J=16.2 Hz), 122.9, 121.28, 121.27, 121.0, 114.10 (d, J=23.6 Hz), 106.9, 57.68 (d, J=2.3 Hz), 57.7, 45.8, 45.0, 38.0, 35.5, 34.1, 12.21 (d, J=7.0 Hz), 12.2. HRMS (ESI-TOF) m/z Calcd for C₂₁H₂₂FN₂O₂⁺[M+H]⁺ 353.1665, found 353.1668.

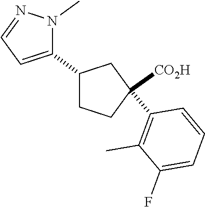 [SMILES: Cc1c(F)cccc1[C@]1(C(=O)O)CC[C@H](c2ccnn2C)C1]

### cis-1-(3-fluoro-2-methylphenyl)-3-(1-methyl-1H-pyrazol-5-yl)cyclopentane-1-carboxylic acid (4al)

Following General Procedure 1 on 0.1 mmol scale. The product was obtained as colorless oil (13.9 mg, 46% yield). ¹H NMR (600 MHz, CDCl₃) δ 7.39 (d, J=2.0 Hz, 1H), 7.23-7.15 (m, 2H), 7.02-6.96 (m, 1H), 6.18 (d, J=2.0 Hz, 1H), 3.78 (s, 3H), 3.24-3.13 (m, 1H), 2.77-2.67 (m, 2H), 2.55 (dd, J=13.5, 8.0 Hz, 1H), 2.39-2.31 (m, 1H), 2.28-2.20 (m, 1H), 2.19 (s, 3H), 1.94-1.83 (m, 1H). ¹³C NMR (151 MHz, CDCl₃) δ 180.3, 162.2 (d, J=243.9), 145.9, 143.8, 138.2, 126.80 (d, J=9.5 Hz), 124.08 (d, J=16.1 Hz), 121.09 (d, J=2.9 Hz), 114.18 (d, J=23.4 Hz), 114.1, 102.9, 57.5, 42.8, 37.4, 36.4, 35.6, 32.0, 12.16 (d, J=7.5 Hz), 12.1. HRMS (ESI-TOF) m/z Calcd for C₁₇H₂₀FN₂O₂⁺[M+H]⁺ 303.1509, found 303.1515.

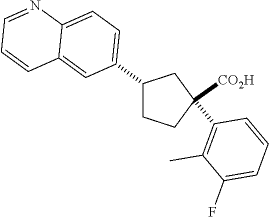 [SMILES: Cc1c(F)cccc1[C@]1(C(=O)O)CC[C@H](c2ccc3ncccc3c2)C1]

### cis-1-(3-fluoro-2-methylphenyl)-3-(quinolin-6-yl)cyclopentane-1-carboxylic acid (4am)

Following General Procedure 1 on 0.1 mmol scale. The product was obtained as colorless oil (22.6 mg, 65% yield). ¹H NMR (600 MHz, DMSO) δ 8.83 (dd, J=4.2, 1.7 Hz, 1H), 8.29 (d, J=7.2 Hz, 1H), 7.98 (d, J=8.7 Hz, 1H), 7.85 (d, J=2.0 Hz, 1H), 7.79 (dd, J=8.8, 2.0 Hz, 1H), 7.49 (dd, J=8.3, 4.2 Hz, 1H), 7.33 (d, J=7.9 Hz, 1H), 7.28-7.22 (m, 1H), 7.08 (t, J=8.9 Hz, 1H), 3.34-3.27 (m, 1H), 2.70-2.60 (m, 2H), 2.56-2.51 (m, 1H), 2.34-2.26 (m, 1H), 2.26-2.17 (m, 1H), 2.12 (d, J=2.6 Hz, 3H), 1.95-1.85 (m, 1H).; ¹³C NMR (151 MHz, DMSO) δ 177.8, 161.38 (d, J=240.6 Hz), 160.6, 150.0, 146.8, 145.4, 143.0, 135.8, 129.7, 129.0, 128.0, 126.87 (d, J=9.4 Hz), 125.2, 123.2, 123.11 (d, J=15.9 Hz), 121.91 (d, J=2.6 Hz), 121.6, 113.30 (d, J=23.1 Hz), 113.2, 57.49 (d, J=2.1 Hz), 44.3, 44.1, 37.4, 33.1, 11.69 (d, J=7.2 Hz); HRMS (ESI-TOF) m/z Calcd for Chemical Formula: C₂₂H₂₁FNO₂⁺[M+H]⁺ 350.1556, found 350.1547.

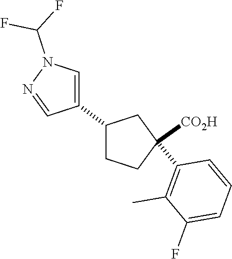 [SMILES: Cc1c(F)cccc1[C@]1(C(=O)O)CC[C@H](c2cnn(C(F)F)c2)C1]

### cis-3-(1-(difluoromethyl)-1H-pyrazol-4-yl)-1-(3-fluoro-2-methylphenyl)cyclopentane-1-carboxylic acid (4an)

Following General Procedure 1 on 0.1 mmol scale. The product was obtained as colorless oil (16.5 mg, 49% yield). ¹H NMR (600 MHz, CDCl₃) δ 7.67 (s, 1H), 7.60 (s, 1H), 7.22-7.15 (m, 2H), 7.01-6.95 (m, 1H), 3.14 (tt, J=10.2, 7.8 Hz, 1H), 2.73-2.65 (m, 1H), 2.62 (dd, J=13.4, 10.2 Hz, 1H), 2.52 (dd, J=13.4, 8.1 Hz, 1H), 2.38-2.30 (m, 1H), 2.27-2.19 (m, 1H), 2.18 (d, J=2.7 Hz, 3H), 1.89-1.79 (m, 1H). ¹³C NMR (151 MHz, CDCl₃) δ 181.4, 161.66 (d, J=243.5 Hz), 143.84 (d, J=3.4 Hz), 141.03 (t, J=2.3 Hz), 128.1, 126.77 (d, J=9.3 Hz), 123.98 (d, J=15.8 Hz), 123.5, 121.18 (d, J=2.9 Hz), 114.16 (d, J=23.7 Hz), 111.14 (t, J=249.8 Hz), 57.6, 57.5, 44.3, 37.7, 34.8, 33.6, 12.2, 12.1. HRMS (ESI-TOF) m/z Calcd for C₁₇H₁₈F₃N₂₀₂⁺[M+H]⁺ 339.1320, found 339.1326.

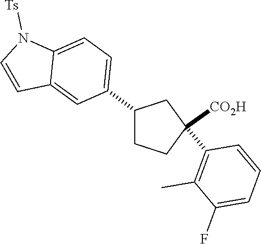 [SMILES: Cc1ccc(S(=O)(=O)n2ccc3cc([C@H]4CC[C@@](C(=O)O)(c5cccc(F)c5C)C4)ccc32)cc1]

### cis-1-(3-fluoro-2-methylphenyl)-3-(1-tosyl-1H-indol-5-yl)cyclopentane-1-carboxylic acid (4ao)

Following General Procedure 1 on 0.1 mmol scale. The product was obtained as colorless oil (30.9 mg, 63% yield). ¹H NMR (600 MHz, CDCl₃) δ 7.91 (d, J=8.5 Hz, 1H), 7.79-7.73 (m, 2H), 7.54 (d, J=3.7 Hz, 1H), 7.45 (d, J=1.7 Hz, 1H), 7.28 (dd, J=8.6, 1.8 Hz, 1H), 7.25-7.16 (m, 4H), 7.02-6.96 (m, 1H), 6.60 (dd, J=3.7, 0.8 Hz, 1H), 3.23 (tt, J=10.9, 7.6 Hz, 1H), 2.78-2.67 (m, 2H), 2.55 (dd, J=13.5, 7.9 Hz, 1H), 2.40-2.33 (m, 1H), 2.33 (s, 3H), 2.24-2.16 (m, 1H), 2.16 (d, J=2.7 Hz, 3H), 2.00-1.91 (m, 1H). ¹³C NMR (151 MHz, CDCl₃) δ 182.5, 162.16 (d, J=243.4 Hz), 145.0, 144.24 (d, J=3.3 Hz), 139.4, 135.5, 133.7, 131.1, 130.0, 127.0, 126.8, 126.7, 124.3, 123.99 (d, J=16.2 Hz) 121.3, 119.6, 114.06 (d, J=23.2 Hz) 113.6, 109.0, 57.63 (d, J=2.5 Hz), 45.3, 45.2, 38.1, 34.2, 21.7, 12.19 (d, J=7.4 Hz). HRMS (ESI-TOF) m/z Calcd for C₂₈H₂₇NOF₄S⁺[M+H]⁺ 492.1645, found 492.1648.

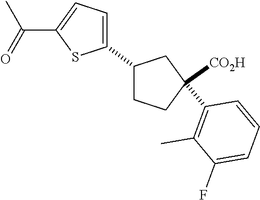 [SMILES: CC(=O)c1ccc([C@H]2CC[C@@](C(=O)O)(c3cccc(F)c3C)C2)s1]

### cis-3-(5-acetylthiophen-2-yl)-1-(3-fluoro-2-methylphenyl)cyclopentane-1-carboxylic acid(4ap)

Following General Procedure 1 on 0.1 mmol scale. The product was obtained as colorless oil (20.1 mg, 58% yield). ¹H NMR (600 MHz, CDCl₃) δ 7.55 (d, J=3.8 Hz, 1H), 7.22-7.13 (m, 2H), 7.00-6.97 (m, 1H), 6.92 (dd, J=3.9, 0.9 Hz, 1H), 3.43 (tt, J=10.2, 7.7 Hz, 1H), 2.79-2.69 (m, 2H), 2.65-2.57 (m, 1H), 2.51 (s, 3H), 2.42-2.25 (m, 2H), 2.17 (d, J=2.7 Hz, 3H), 2.03-1.94 (m, 1H). ¹³C NMR (151 MHz, CDCl₃) δ 190.8, 181.8, 162.17 (d, J=243.8 Hz) 158.3, 143.48 (d, J=3.4 Hz), 142.1, 133.0, 126.81 (d, J=9.2 Hz), 124.8, 123.95 (d, J=16.3 Hz), 121.18 (d, J=2.9 Hz), 114.24 (d, J=23.5 Hz), 57.66 (d, J=2.3 Hz), 45.0, 40.7, 37.6, 34.4, 26.6, 12.17 (d, J=7.4 Hz). HRMS (ESI-TOF) m/z Calcd for C₁₉H₂₀FO₃S⁺[M+H]⁺ 347.1117, found 347.1114.

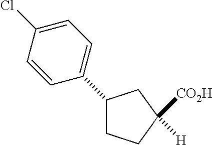

### cis-3-(4-chlorophenyl)cyclopentane-1-carboxylic acid (4aq)

Following General Procedure 1 on 0.1 mmol scale. The product was obtained as colorless oil (12.1 mg, 54% yield). ¹H NMR (600 MHz, CDCl₃) δ 7.28-7.24 (m, 2H), 7.21-7.17 (m, 2H), 3.06 (tt, J=10.9, 7.0 Hz, 1H), 2.98 (tdd, J=9.6, 7.9, 6.0 Hz, 1H), 2.43-2.36 (m, 1H), 2.18-2.08 (m, 2H), 2.08-2.02 (m, 1H), 1.92 (ddd, J=12.8, 11.4, 9.6 Hz, 1H), 1.79-1.71 (m, 1H); ¹³C NMR (151 MHz, CDCl₃) δ 182.1, 142.9, 132.0, 128.6, 128.6, 45.7, 43.5, 38.2, 34.0, 29.4; HRMS (ESI-TOF) m/z Calcd for C₁₂H₁₂ClO₂⁻ [symbol] ^{[M-H]}⁻ 223.0526, found 223.0527.

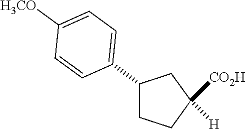 [SMILES: COc1ccc([C@H]2CC[C@H](C(=O)O)C2)cc1]

### cis-3-(4-methoxyphenyl)cyclopentane-1-carboxylic acid (4ar)

Following General Procedure 1 on 0.1 mmol scale. The product was obtained as colorless oil (8.1 mg, 37% yield). ¹H NMR (600 MHz, CDCl₃) δ 7.21-7.16 (m, 2H), 6.87-6.83 (m, 2H), 3.79 (s, 3H), 3.04 (tt, J=11.1, 6.9 Hz, 1H), 2.97 (tdd, J=9.7, 7.7, 6.1 Hz, 1H), 2.42-2.34 (m, 1H), 2.17-2.00 (m, 3H), 1.92 (ddd, J=12.7, 11.5, 9.8 Hz, 1H), 1.80-1.71 (m, 1H); ¹³C NMR (151 MHz, CDCl₃) δ 182.7, 158.1, 136.4, 128.1, 113.9, 55.4, 45.6, 43.5, 38.4, 34.2, 29.3; HRMS (ESI-TOF) m/z Calcd for C₁₃H₁₅₀₃—[M−H]⁻ 219.1021, found 219.1028.

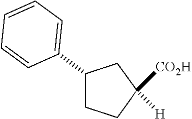 [SMILES: O=C(O)[C@H]1CC[C@H](c2ccccc2)C1]

### cis-3-phenylcyclopentane-1-carboxylic acid (4as)

Following General Procedure 1 on 0.1 mmol scale. The product was obtained as colorless oil (7.2 mg, 38% yield). ¹H NMR (600 MHz, CDCl₃) δ 7.33-7.23 (m, 4H), 7.23-7.17 (m, 1H), 3.09 (tt, J=11.1, 7.0 Hz, 1H), 3.03-2.95 (m, 1H), 2.45-2.38 (m, 1H), 2.19-2.09 (m, 2H), 2.10-2.02 (m, 1H), 2.01-1.93 (m, 1H), 1.85-1.75 (m, 1H). ¹³C NMR (151 MHz, CDCl₃) δ 181.3, 144.4, 128.5, 127.2, 126.4, 46.4, 43.4, 38.3, 34.0, 29.4. HRMS (ESI-TOF) m/z Calcd for C₁₂H₁₃O₂⁻ [M−H]⁻ 189.0916, found 189.0925.

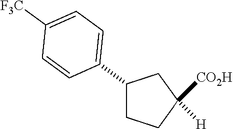 [SMILES: O=C(O)[C@H]1CC[C@H](c2ccc(C(F)(F)F)cc2)C1]

### cis-3-(4-(trifluoromethyl)phenyl)cyclopentane-1-carboxylic acid (4at)

Following General Procedure 1 on 0.1 mmol scale. The product was obtained as colorless oil (18.1 mg, 70% yield). ¹H NMR (600 MHz, CDCl₃) δ 7.59-7.52 (m, 2H), 7.40-7.35 (m, 2H), 3.15 (tt, J=10.9, 7.0 Hz, 1H), 3.02 (tdd, J=9.6, 7.8, 5.9 Hz, 1H), 2.47-2.40 (m, 1H), 2.21-2.12 (m, 2H), 2.08 (ddt, J=14.0, 10.0, 7.8 Hz, 1H), 1.98 (ddd, J=12.9, 11.3, 9.5 Hz, 1H), 1.85-1.76 (m, 1H); ¹³C NMR (151 MHz, CDCl₃) δ 182.4, 148.5, 128.70 (d, J=32.3 Hz), 127.6, 125.47 (d, J=3.9 Hz), 124.41 (d, J=271.3 Hz), 46.1, 43.5, 38.0, 34.0, 29.5; HRMS (ESI-TOF) m/z Calcd for C₁₃H₁₂F₃O₂⁻ [M−H]⁻ 257.0789, found 257.0789.

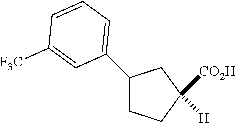 [SMILES: O=C(O)[C@H]1CCC(c2cccc(C(F)(F)F)c2)C1]

### cis-3-(3-(trifluoromethyl)phenyl)cyclopentane-1-carboxylic acid (4au)

Following General Procedure 1 on 0.1 mmol scale. The product was obtained as colorless oil (16.0 mg, 62% yield). ¹H NMR (600 MHz, CDCl₃) δ 7.51 (td, J=1.8, 0.9 Hz, 1H), 7.48-7.44 (m, 2H), 7.43-7.39 (m, 1H), 3.15 (tt, J=11.1, 7.0 Hz, 1H), 3.02 (tdd, J=9.7, 7.7, 6.1 Hz, 1H), 2.48-2.40 (m, 1H), 2.21-2.13 (m, 2H), 2.12-2.04 (m, 1H), 1.98 (ddd, J=12.8, 11.4, 9.6 Hz, 1H), 1.85-1.77 (m, 1H); ¹³C NMR (151 MHz, CDCl₃) δ 182.4, 145.3, 130.81 (d, J=31.9 Hz), 130.59 (d, J=1.6 Hz), 129.0, 124.37 (q, J=272.2 Hz), 124.00 (q, J=3.6 Hz), 123.25 (q, J=3.9 Hz), 46.1, 43.5, 38.0, 33.9, 29.3; HRMS (ESI-TOF) m/z Calcd for C₁₃H₁₂F₃O₂⁻ [M−H]⁻ 257.0789, found 257.0792.

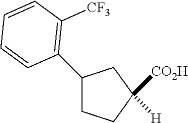 [SMILES: O=C(O)[C@H]1CCC(c2ccccc2C(F)(F)F)C1]

### cis-3-(2-(trifluoromethyl)phenyl)cyclopentane-1-carboxylic acid (4av)

Following General Procedure 1 on 0.1 mmol scale. The product was obtained as colorless oil (13.2 mg, 51% yield). ¹H NMR (600 MHz, CDCl₃) δ 7.64-7.56 (m, 2H), 7.55-7.49 (m, 1H), 7.29 (t, J=7.6 Hz, 1H), 3.52-3.42 (m, 1H), 3.07-2.98 (m, 1H), 2.47-2.37 (m, 1H), 2.25-2.16 (m, 1H), 2.17-2.05 (m, 2H), 2.04-1.96 (m, 1H), 1.88-1.78 (m, 1H); ¹³C NMR (151 MHz, CDCl₃) δ 182.1, 143.8, 132.3, 128.65 (q, J=29.2 Hz), 128.0, 126.2, 125.73 (q, J=5.9 Hz), 124.77 (q, J=276.1 Hz), 43.6, 41.4, 39.3, 35.1, 30.0. HRMS (ESI-TOF) m/z Calcd for C₁₃H₁₂F₃O₂⁻ [M−H]⁻ 257.0789, found 257.0798.

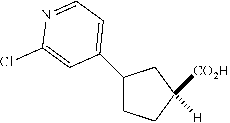 [SMILES: O=C(O)[C@H]1CCC(c2ccnc(Cl)c2)C1]

### cis-3-(2-chloropyridin-4-yl)cyclopentane-1-carboxylic acid (4aw)

Following General Procedure 1 on 0.1 mmol scale. The product was obtained as colorless oil (10.1 mg, 45% yield). ¹H NMR (600 MHz, CDCl₃) δ 8.30 (dd, J=5.3, 0.6 Hz, 1H), 7.23 (dt, J=1.5, 0.7 Hz, 1H), 7.12 (ddd, J=5.2, 1.6, 0.6 Hz, 1H), 3.12-2.97 (m, 2H), 2.43 (dtd, J=12.8, 7.5, 1.1 Hz, 1H), 2.20-2.12 (m, 2H), 2.12-2.03 (m, 1H), 1.96 (ddd, J=12.9, 11.0, 9.3 Hz, 1H), 1.83-1.73 (m, 1H); ¹³C NMR (151 MHz, CDCl₃) δ 181.0, 157.2, 151.8, 149.7, 123.2, 121.6, 45.2, 43.4, 37.1, 33.3, 29.5; HRMS (ESI-TOF) m/z Calcd for C₁₁H₁₃ClNO₂⁺[M+H]⁺ 226.0635, found 226.0635.

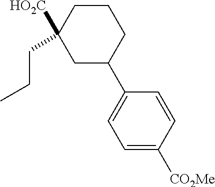

### cis-3-(4-(methoxycarbonyl)phenyl)-1-propylcyclohexane-1-carboxylic acid (5a)

Following General Procedure 2 on 0.1 mmol scale. The product was obtained as colorless oil (20.0 mg, 65% yield). ¹H NMR (600 MHz, CDCl₃) δ 7.98-7.93 (m, 2H), 7.29-7.26 (m, 2H), 3.90 (s, 3H), 2.78 (tt, J=12.6, 3.5 Hz, 1H), 2.01 (dq, J=13.5, 2.5 Hz, 1H), 1.94-1.85 (m, 2H), 1.81-1.71 (m, 4H), 1.67-1.57 (m, 2H), 1.43-1.34 (m, 1H), 1.32-1.25 (m, 2H), 0.96 (t, J=7.3 Hz, 3H); ¹³C NMR (151 MHz, CDCl₃) δ 183.4, 167.2, 152.1, 129.9, 128.3, 127.1, 52.1, 46.3, 39.0, 39.0, 34.8, 33.3, 31.6, 21.6, 18.0, 14.8; HRMS (ESI-TOF) m/z Calcd for C₁₈H₂₅O₄⁺[M+H]⁺ 305.1753, found 305.1766.

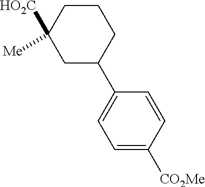 [SMILES: COC(=O)c1ccc(C2CCC[C@](C)(C(=O)O)C2)cc1]

### cis-3-(4-(methoxycarbonyl)phenyl)-1-methylcyclohexane-1-carboxylic acid (5b)

Following General Procedure 2 on 0.1 mmol scale with the addition of 0.1 mL CH₃CN. The product was obtained as white solid (17.1 mg, 62% yield). ¹H NMR (600 MHz, CDCl₃) δ 7.99-7.93 (m, 2H), 7.30-7.26 (m, 2H), 3.90 (s, 3H), 2.80 (tt, J=11.9, 4.3 Hz, 1H), 1.92-1.84 (m, 3H), 1.84-1.70 (m, 3H), 1.64 (qt, J=13.1, 3.7 Hz, 1H), 1.37 (s, 4H); ¹³C NMR (151 MHz, CDCl₃) δ 184.0, 167.2, 152.0, 130.0, 128.3, 127.1, 52.1, 42.6, 40.8, 39.2, 33.4, 33.1, 21.7, 20.2; HRMS (ESI-TOF) m/z Calcd for C₁₆H₂₁O₄⁺[M+H]⁺ 277.1440, found 277.1440.

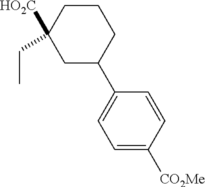

### cis-1-ethyl-3-(4-(methoxycarbonyl)phenyl)cyclohexane-1-carboxylic acid (5c)

Following General Procedure 2 on 0.1 mmol scale. The product was obtained as white solid (17.1 mg, 59% yield). ¹H NMR (600 MHz, CDCl₃) δ 7.98-7.92 (m, 2H), 7.29-7.26 (m, 2H), 3.90 (s, 3H), 2.75 (tt, J=12.5, 3.2 Hz, 1H), 2.05-1.96 (m, 1H), 1.95-1.81 (m, 4H), 1.79-1.71 (m, 2H), 1.68-1.54 (m, 2H), 1.43-1.32 (m, 1H), 0.89 (t, J=7.4 Hz, 3H); ¹³C NMR (151 MHz, CDCl₃) δ 183.6, 167.3, 152.1, 129.9, 128.3, 127.1, 52.2, 46.6, 39.0, 38.5, 33.3, 31.2, 25.2, 21.6, 9.1; HRMS (ESI-TOF) m/z Calcd for C₁₇H₂₃O₄⁺[M+H]⁺ 291.1596, found 291.1609.

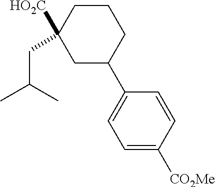

### cis-1-isobutyl-3-(4-(methoxycarbonyl)phenyl)cyclohexane-1-carboxylic acid (5d)

Following General Procedure 2 on 0.1 mmol scale. The product was obtained as colorless oil (19.4 mg, 61% yield). ¹H NMR (600 MHz, CDCl₃) δ 7.98-7.94 (m, 2H), 7.29-7.26 (m, 2H), 3.90 (s, 3H), 2.77 (tt, J=12.6, 3.4 Hz, 1H), 2.03-1.97 (m, 1H), 1.92-1.85 (m, 2H), 1.85-1.73 (m, 4H), 1.70-1.58 (m, 3H), 1.39 (qd, J=12.5, 4.4 Hz, 1H), 0.94 (d, J=6.6 Hz, 3H), 0.91 (d, J=6.6 Hz, 3H); ¹³C NMR (151 MHz, CDCl₃) δ 184.9, 167.2, 152.1, 130.0, 128.3, 127.1, 52.1, 45.6, 40.6, 39.1(2), 33.3, 32.3, 25.0, 24.0, 23.6, 21.7; HRMS (ESI-TOF) m/z Calcd for C₁₉H₂₇O₄⁺ [M+H]⁺ 319.1909, found 319.1922.

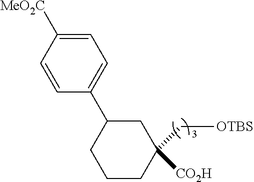

### cis-1-(3-((tert-butyldimethylsilyl)oxy)propyl)-3-(4-(methoxycarbonyl)phenyl)cyclohexane-1-carboxylic acid (5e)

Following General Procedure 2 on 0.1 mmol scale. The product was obtained as colorless oil (27.9 mg, 64% yield). ¹H NMR (600 MHz, CDCl₃) δ 7.98-7.94 (m, 2H), 7.27 (d, J=8.4 Hz, 2H), 3.90 (s, 3H), 3.64 (td, J=6.2, 1.4 Hz, 2H), 2.77 (ddt, J=16.0, 12.6, 3.4 Hz, 1H), 2.01-1.98 (m, 1H), 1.94-1.80 (m, 4H), 1.80-1.73 (m, 2H), 1.69-1.57 (m, 2H), 1.52-1.45 (m, 2H), 1.43-1.34 (m, 1H), 0.91 (s, 9H), 0.06 (s, 6H); ¹³C NMR (151 MHz, CDCl₃) δ 183.5, 167.3, 152.0, 129.9, 128.3, 127.1, 63.3, 52.2, 45.9, 38.95, 38.94, 33.3, 31.7, 28.7, 28.1, 26.1, 21.6, 18.5, −5.1; HRMS (ESI-TOF) m/z Calcd for C₂₄H₃₉O₅Si⁺ [M+H]⁺ 435.2567, found 435.2563.

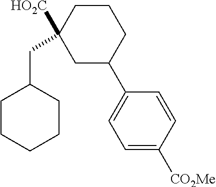

### cis-1-(cyclohexylmethyl)-3-(4-(methoxycarbonyl)phenyl)cyclohexane-1-carboxylic acid (5f)

Following General Procedure 2 on 0.1 mmol scale. The product was obtained as colorless oil (17.5 mg, 49% yield). ¹H NMR (600 MHz, CDCl₃) δ 7.99-7.94 (m, 2H), 7.30-7.26 (m, 2H), 3.90 (s, 3H), 2.78 (tt, J=12.6, 3.4s Hz, 1H), 2.02-1.95 (m, 1H), 1.93-1.84 (m, 2H), 1.82-1.72 (m, 4H), 1.69-1.57 (m, 7H), 1.43-1.34 (m, 1H), 1.34-1.08 (m, 4H), 1.05-0.94 (m, 2H); ¹³C NMR (151 MHz, CDCl₃) δ 184.5, 167.2, 152.1, 130.0, 128.3, 127.1, 52.2, 45.5, 39.3, 39.1, 34.5, 34.3, 33.4, 32.2, 26.5, 26.5, 26.4, 21.7; HRMS (ESI-TOF) m/z Calcd for C₂₂H₃₁O₄⁺ [M+H]⁺ 359.2222, found 359.2233.

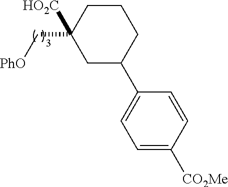

### cis-3-(4-(methoxycarbonyl)phenyl)-1-(3-phenoxypropyl)cyclohexane-1-carboxylic acid (5g)

Following General Procedure 2 on 0.1 mmol scale. The product was obtained as colorless oil (21.0 mg, 53% yield). ¹H NMR (600 MHz, CDCl₃) δ 7.98-7.94 (m, 2H), 7.30-7.26 (m, 3H), 7.25 (d, J=0.5 Hz, 1H), 6.94 (tt, J=7.4, 1.1 Hz, 1H), 6.92-6.89 (m, 2H), 4.00 (td, J=6.3, 3.3 Hz, 2H), 3.90 (s, 3H), 2.79 (tt, J=12.7, 3.4 Hz, 1H), 2.06-1.96 (m, 3H), 1.92 (td, J=19.5, 18.9, 4.8 Hz, 2H), 1.84-1.74 (m, 4H), 1.73-1.60 (m, 2H), 1.42 (qd, J=12.4, 3.7 Hz, 1H); ¹³C NMR (151 MHz, CDCl₃) δ 183.3, 167.2, 159.0, 151.9, 130.0, 129.6, 128.3, 127.1, 120.8, 114.6, 67.9, 52.2, 46.1, 39.0, 38.9, 33.2, 31.6, 28.9, 24.7, 21.5; HRMS (ESI-TOF) m/z Calcd for C₂₄H₂₇O₅⁻ [M−H]⁻ 395.1858, found 395.1857.

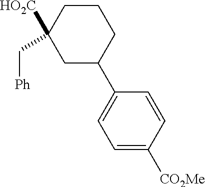

### cis-1-benzyl-3-(4-(methoxycarbonyl)phenyl)cyclohexane-1-carboxylic acid (5h)

Following General Procedure 2 on 0.1 mmol scale. The product was obtained as colorless oil (18.1 mg, 51% yield). ¹H NMR (600 MHz, CDCl₃) δ 7.99-7.95 (m, 2H), 7.30-7.28 (m, 2H), 7.28-7.24 (m, 2H), 7.23-7.18 (m, 3H), 3.90 (s, 3H), 3.18 (d, J=2.3 Hz, 2H), 3.01 (tt, J=12.7, 3.4 Hz, 1H), 2.03-1.94 (m, 3H), 1.91-1.80 (m, 2H), 1.75 (dd, J=13.9, 12.8 Hz, 1H), 1.67 (td, J=13.3, 5.0 Hz, 1H), 1.47 (qd, J=12.6, 4.6 Hz, 1H); ¹³C NMR (151 MHz, CDCl₃) δ 182.1, 167.2, 151.7, 137.6, 130.0, 129.8, 128.5, 128.4, 127.0, 126.9, 52.2, 47.8, 39.1, 38.7, 38.5, 33.1, 31.5, 21.8; HRMS (ESI-TOF) m/z Calcd for C₂₂H₂₅O₄⁺[M+H]⁺ 353.1753, found 353.1763.

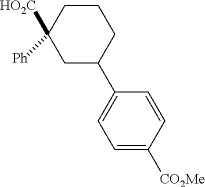 [SMILES: COC(=O)c1ccc(C2CCC[C@@](C(=O)O)(c3ccccc3)C2)cc1]

### cis-3-(4-(methoxycarbonyl)phenyl)-1-phenylcyclohexane-1-carboxylic acid (5i)

Following General Procedure 2 on 0.1 mmol scale. The product was obtained as colorless oil (15.9 mg, 47% yield). ¹H NMR (600 MHz, CDCl₃) δ 7.98-7.93 (m, 2H), 7.53-7.48 (m, 2H), 7.43-7.38 (m, 2H), 7.32-7.28 (m, 1H), 7.26-7.23 (m, 2H), 3.90 (s, 3H), 2.89-2.77 (m, 2H), 2.63 (tt, J=12.3, 3.2 Hz, 1H), 2.01 (dd, J=14.0, 12.8 Hz, 1H), 1.86 (ddd, J=14.1, 13.2, 3.6 Hz, 1H), 1.83-1.75 (m, 2H), 1.57-1.41 (m, 2H); ¹³C NMR (151 MHz, CDCl₃) δ 180.9, 167.2, 151.9, 138.6, 130.0, 129.0, 128.3, 127.9, 127.3, 127.0, 52.2, 50.0, 39.5, 39.2, 33.7, 31.9, 22.0; HRMS (ESI-TOF) m/z Calcd for C₂₁H₂₃O₄⁺[M+H]⁺ 339.1596, found 339.1591.

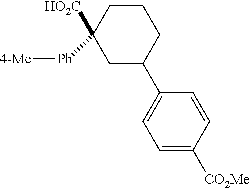

### cis-3-(4-(methoxycarbonyl)phenyl)-1-(p-tolyl)cyclohexane-1-carboxylic acid (5j)

Following General Procedure 2 on 0.1 mmol scale. The product was obtained as colorless oil (19.7 mg, 56% yield). ¹H NMR (600 MHz, CDCl₃) δ 7.98-7.94 (m, 2H), 7.41-7.37 (m, 2H), 7.26-7.19 (m, 4H), 3.90 (s, 3H), 2.88-2.74 (m, 2H), 2.63 (tt, J=12.3, 3.1 Hz, 1H), 2.36 (s, 3H), 1.98 (dd, J=13.9, 12.7 Hz, 1H), 1.89-1.73 (m, 3H), 1.57-1.39 (m, 2H); ¹³C NMR (151 MHz, CDCl₃) δ 181.0, 167.2, 152.0, 137.0, 135.6, 130.0, 129.8, 128.3, 127.7, 127.0, 52.2, 49.7, 39.5, 39.2, 33.7, 31.8, 22.0, 21.1; HRMS (ESI-TOF) m/z Calcd for C₂₂H₂₅O₄⁺[M+H]⁺ 353.1753, found 353.1740.

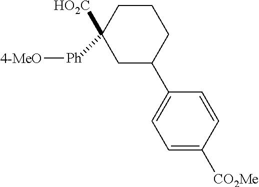

### cis-3-(4-(methoxycarbonyl)phenyl)-1-(4-methoxyphenyl)cyclohexane-1-carboxylic acid (5k)

Following General Procedure 2 on 0.1 mmol scale. The product was obtained as colorless oil (20.2 mg, 55% yield). ¹H NMR (600 MHz, CDCl₃) δ 7.96 (d, J=8.3 Hz, 2H), 7.44-7.39 (m, 2H), 7.26-7.23 (m, 2H), 6.96-6.91 (m, 2H), 3.90 (s, 3H), 3.82 (s, 3H), 2.85-2.73 (m, 2H), 2.63 (tt, J=12.3, 3.1 Hz, 1H), 1.98 (dd, J=13.9, 12.7 Hz, 1H), 1.88-1.74 (m, 3H), 1.58-1.40 (m, 2H); ¹³C NMR (151 MHz, CDCl₃) δ 181.1, 167.2, 158.7, 151.9, 130.5, 130.0, 129.0, 128.3, 127.0, 114.4, 55.4, 52.2, 49.3, 39.6, 39.2, 33.8, 31.9, 22.0; HRMS (ESI-TOF) m/z Calcd for C₂₂H₂₅O₅⁺ [M+H]⁺ 369.1702, found 369.1700.

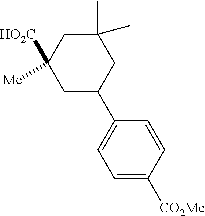 [SMILES: COC(=O)c1ccc(C2CC(C)(C)C[C@](C)(C(=O)O)C2)cc1]

### cis-5-(4-(methoxycarbonyl)phenyl)-1,3,3-trimethylcyclohexane-1-carboxylic acid (51)

Following General Procedure 2 on 0.1 mmol scale. The product was obtained as colorless oil (11.6 mg, 38% yield). ¹H NMR (600 MHz, CDCl₃) δ 8.00-7.93 (m, 2H), 7.34-7.27 (m, 2H), 3.90 (s, 3H), 2.95 (tt, J=12.4, 3.6 Hz, 1H), 1.90-1.79 (m, 2H), 1.68-1.58 (m, 3H), 1.46 (s, 3H), 1.34 (t, J=12.9 Hz, 1H), 1.15 (s, 3H), 1.00 (s, 3H); ¹³C NMR (151 MHz, CDCl₃) δ 184.4, 167.2, 151.9, 130.0, 128.3, 127.3, 52.2, 46.7, 45.6, 43.3, 40.5, 36.2, 35.2, 31.9, 28.0, 23.2; HRMS (ESI-TOF) m/z Calcd for C₁₈H₂₅O₄⁺[M+H]⁺ 305.1753, found 305.1742.

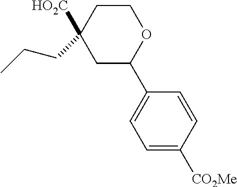

### cis-2-(4-(methoxycarbonyl)phenyl)-4-propyltetrahydro-2H-pyran-4-carboxylic acid (5m)

Following General Procedure 2 on 0.1 mmol scale. The product was obtained as colorless oil (11.9 mg, 39% yield). ¹H NMR (600 MHz, CDCl₃) δ 8.03-7.97 (m, 2H), 7.44-7.37 (m, 2H), 4.58 (dd, J=11.9, 2.2 Hz, 1H), 4.06 (ddd, J=12.0, 5.2, 1.7 Hz, 1H), 3.91 (s, 3H), 3.81 (ddd, J=12.9, 12.0, 2.2 Hz, 1H), 2.04 (ddd, J=14.0, 12.9, 5.2 Hz, 1H), 1.97 (dt, J=14.0, 2.3 Hz, 1H), 1.94-1.87 (m, 2H), 1.82 (dd, J=14.0, 11.8 Hz, 1H), 1.76 (dq, J=13.7, 2.0 Hz, 1H), 1.39-1.28 (m, 2H), 0.99 (t, J=7.2 Hz, 3H); ¹³C NMR (151 MHz, CDCl₃) δ 182.3, 167.1, 147.6, 129.9, 129.5, 125.8, 74.4, 63.7, 52.2, 44.2, 39.6, 34.6, 31.1, 18.0, 14.7; HRMS (ESI-TOF) m/z Calcd for C₁₇H₂₃O₅⁺[M+H]⁺ 307.1545, found 307.1543.

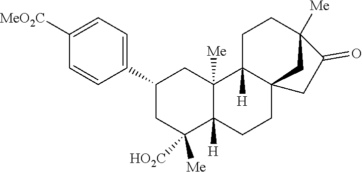

### (2R,4R,4aS,6aR,9S,11aR,11bS)-2-(4-(methoxycarbonyl)phenyl)-4,9,11b-trimethyl-8-oxotetradecahydro-6a,9-methanocyclohepta[a]naphthalene-4-carboxylic acid (5n)

Following General Procedure 2 on 0.1 mmol scale. The product was obtained as colorless oil (28.4 mg, 63% yield). ¹H NMR (600 MHz, CDCl₃) δ 7.99-7.94 (m, 2H), 7.30-7.26 (m, 2H), 3.90 (s, 3H), 2.92-2.84 (m, 1H), 2.73 (t, J=13.8 Hz, 1H), 2.64 (dd, J=18.6, 3.7 Hz, 1H), 1.98-1.90 (m, 1H), 1.81 (d, J=18.7 Hz, 1H), 1.74 (ddd, J=14.5, 7.4, 1.7 Hz, 1H), 1.68 (dt, J=13.8, 3.1 Hz, 1H), 1.63-1.50 (m, 5H), 1.47-1.40 (m, 4H), 1.38 (s, 3H), 1.35-1.24 (m, 3H), 1.08 (s, 3H), 0.97 (s, 3H); ¹³C NMR (151 MHz, CDCl₃) δ 223.0, 182.2, 167.2, 152.2, 130.0, 128.3, 127.2, 55.8, 54.2, 52.2, 51.4, 49.1, 48.3, 48.1, 44.9, 40.7, 39.8, 37.8, 37.4, 37.2, 36.1, 27.0, 22.5, 20.9, 20.3, 19.9; HRMS (ESI-TOF) m/z Calcd for C₂₈H₃₇O₅s [M+H]⁺ 453.2641, found 453.2625.

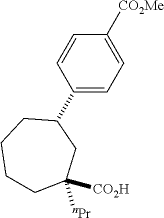

### cis-3-(4-(methoxycarbonyl)phenyl)-1-propylcycloheptane-1-carboxylic acid (6a)

Following General Procedure 3 on 0.1 mmol scale. The product was obtained as a colorless oil (16.5 mg, 52% yield). ¹H NMR (600 MHz, CDCl₃) δ 7.98-7.93 (m, 2H), 7.25-7.22 (m, 2H), 3.90 (s, 3H), 2.74 (t, J=10.7 Hz, 1H), 2.33 (td, J=14.8, 14.3, 9.4 Hz, 2H), 1.94-1.90 (m, 2H), 1.80-1.73 (m, 1H), 1.71-1.55 (m, 3H), 1.55-1.37 (m, 3H), 1.30-1.17 (m, 3H), 0.89 (t, J=7.3 Hz, 3H); ¹³C NMR (151 MHz, CDCl₃) δ 183.9, 167.2, 155.1, 130.1, 127.9, 126.6, 52.1, 49.2, 42.9, 42.1, 41.1, 39.3, 36.5, 30.7, 23.9, 18.2, 14.7; HRMS (ESI-TOF) m/z Calcd for C₁₉H₂₇O₄⁺[M+H]⁺ 319.1909, found 319.1917. A crystal suitable for X-ray diffraction was grown by slow evaporation from a mixture of hexanes and CHCl₃.

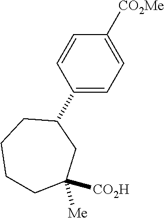

### cis-3-(4-(methoxycarbonyl)phenyl)-1-methylcycloheptane-1-carboxylic acid (6b)

Following General Procedure 3 on 0.1 mmol scale. The product was obtained as a colorless oil (15.1 mg, 52% yield). ¹H NMR (600 MHz, CDCl₃) δ 7.97-7.94 (m, 2H), 7.26-7.22 (m, 2H), 3.90 (s, 3H), 2.76 (t, J=10.7 Hz, 1H), 2.42 (dd, J=14.4, 10.4 Hz, 1H), 2.30 (dd, J=14.7, 8.7 Hz, 1H), 1.98-1.89 (m, 2H), 1.86-1.80 (m, 1H), 1.78-1.53 (m, 3H), 1.52-1.38 (m, 2H), 1.29 (s, 3H); ¹³C NMR (151 MHz, CDCl₃) δ 183.8, 167.2, 155.0, 130.1, 127.9, 126.6, 52.1, 45.1, 44.6, 41.1, 39.1, 38.3, 30.6, 25.9, 24.0; HRMS (ESI-TOF) m/z Calcd for C₁₇H₂₃O₄+[M+H]⁺ 291.1596, found 291.1599.

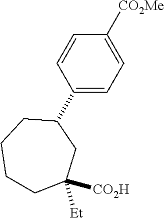

### cis-1-ethyl-3-(4-(methoxycarbonyl)phenyl)cycloheptane-1-carboxylic acid (6c)

Following General Procedure 3 on 0.1 mmol scale. The product was obtained as a colorless oil (17.0 mg, 56% yield). ¹H NMR (600 MHz, CDCl₃) δ 7.98-7.92 (m, 2H), 7.24 (d, J=8.2 Hz, 2H), 3.90 (s, 3H), 2.72 (t, J=10.7 Hz, 1H), 2.33 (td, J=14.7, 14.1, 9.4 Hz, 2H), 1.92 (t, J=13.8 Hz, 3H), 1.76 (dq, J=14.7, 7.1 Hz, 2H), 1.70-1.61 (m, 2H), 1.58-1.34 (m, 3H), 0.84 (t, J=7.4 Hz, 3H); ¹³C NMR (151 MHz, CDCl₃) δ 183.4, 167.3, 155.1, 130.1, 127.9, 126.7, 52.2, 49.6, 42.3, 41.1, 39.3, 36.3, 32.4, 30.8, 23.9, 9.4; HRMS (ESI-TOF) m/z Calcd for C₁₈H₂₅O₄⁺[M+H]305.1753, found 305.1757.

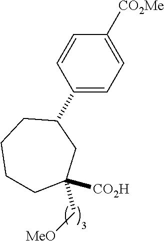

### cis-3-(4-(methoxycarbonyl)phenyl)-1-(3-methoxypropyl)cycloheptane-1-carboxylic acid (6d)

Following General Procedure 3 on 0.1 mmol scale. The product was obtained as a colorless oil (17.4 mg, 50% yield). ¹H NMR (600 MHz, CDCl₃) δ 7.95 (d, J=8.0 Hz, 2H), 7.24 (d, J=8.0 Hz, 2H), 3.90 (s, 3H), 3.34 (tq, J=6.7, 3.0 Hz, 2H), 3.30 (s, 3H), 2.74 (t, J=10.7 Hz, 1H), 2.34 (ddd, J=14.1, 9.5, 3.1 Hz, 2H), 1.91 (t, J=13.8 Hz, 3H), 1.76 (ddd, J=16.6, 10.4, 4.0 Hz, 2H), 1.71-1.60 (m, 2H), 1.57-1.35 (m, 5H); ¹³C NMR (151 MHz, CDCl₃) δ 183.4, 167.2, 155.0, 130.1, 127.9, 126.6, 73.0, 58.6, 52.1, 48.9, 42.9, 41.1, 39.3, 36.5, 36.1, 30.7, 25.1, 23.9; HRMS (ESI-TOF) m/z Calcd for C₂₀H₂₈O₅Na⁺ [M+Na]371.1834, found 371.1826.

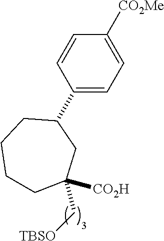

### cis-1-(3-((tert-butyldimethylsilyl)oxy)propyl)-3-(4-(methoxycarbonyl)phenyl)cycloheptane-1-carboxylic acid (6e)

Following General Procedure 3 on 0.1 mmol scale. The product was obtained as a colorless oil (19.7 mg, 44% yield). ¹H NMR (600 MHz, CDCl₃) δ 7.97-7.92 (m, 2H), 7.25-7.21 (m, 2H), 3.90 (s, 3H), 3.57 (t, J=6.3 Hz, 2H), 2.74 (t, J=10.7 Hz, 1H), 2.33 (ddd, J=14.7, 9.5, 7.4 Hz, 2H), 1.91 (dd, J=14.0, 9.6 Hz, 3H), 1.81-1.71 (m, 2H), 1.71-1.61 (m, 2H), 1.56-1.38 (m, 5H), 0.88 (s, 9H), 0.02 (s, 6H); ¹³C NMR (151 MHz, CDCl₃) δ 183.7, 167.3, 155.0, 130.1, 127.9, 126.6, 63.4, 52.1, 48.9, 42.9, 41.1, 39.1, 36.6, 35.8, 30.7, 28.3, 26.1, 23.9, 18.5, −5.1; HRMS (ESI-TOF) m/z Calcd for C₂₅H₄₁O₅Si⁺[M+H]449.2723, found 449.2715.

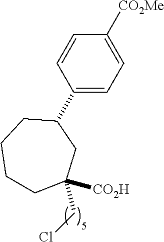

### cis-1-(5-chloropentyl)-3-(4-(methoxycarbonyl)phenyl)cycloheptane-1-carboxylic acid (6f)

Following General Procedure 3 on 0.1 mmol scale. The product was obtained as a colorless oil (19.7 mg, 52% yield). ¹H NMR (600 MHz, CDCl₃) δ 7.96 (d, J=7.9 Hz, 2H), 7.23 (d, J=8.1 Hz, 2H), 3.90 (s, 3H), 3.49 (t, J=6.6 Hz, 2H), 2.73 (t, J=10.7 Hz, 1H), 2.39-2.29 (m, 2H), 1.91 (dd, J=14.7, 7.3 Hz, 3H), 1.83-1.59 (m, 7H), 1.57-1.35 (m, 6H); ¹³C NMR (151 MHz, CDCl₃) δ 183.6, 167.2, 155.0, 130.2, 128.0, 126.6, 52.2, 49.1, 45.1, 42.8, 41.1, 39.4, 39.2, 36.6, 32.4, 30.7, 27.4, 24.2, 23.9; HRMS (ESI-TOF) m/z Calcd for C₂₁H₂₈ClO₄⁻ [M−H]⁻ 379.1676, found 379.1674.

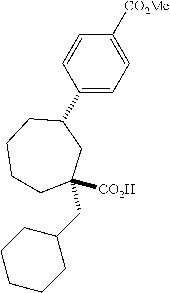

### cis-1-(cyclohexylmethyl)-3-(4-(methoxycarbonyl)phenyl)cycloheptane-1-carboxylic acid (6g)

Following General Procedure 3 on 0.1 mmol scale. The product was obtained as a colorless oil (20.1 mg, 54% yield). ¹H NMR (600 MHz, CDCl₃) δ 7.98-7.92 (m, 2H), 7.23 (d, J=7.9 Hz, 2H), 3.90 (d, J=1.2 Hz, 3H), 2.73 (t, J=10.6 Hz, 1H), 2.35 (dd, J=14.6, 8.7 Hz, 1H), 2.27 (dd, J=14.5, 10.0 Hz, 1H), 1.90 (dd, J=19.3, 14.3 Hz, 3H), 1.80-1.73 (m, 1H), 1.69-1.45 (m, 9H), 1.40 (dt, J=13.9, 10.0 Hz, 2H), 1.32-1.27 (m, 1H), 1.20-1.02 (m, 3H), 0.98-0.82 (m, 2H); ¹³C NMR (151 MHz, CDCl₃) δ 184.6, 167.3, 155.0, 130.1, 127.9, 126.6, 52.1, 48.2, 47.0, 43.7, 41.0, 39.3, 36.5, 34.7, 34.3, 34.0, 30.8, 26.5, 26.5, 26.3, 23.8; HRMS (ESI-TOF) m/z Calcd for C₂₃H₃₃O₄⁺[M+H]373.2379, found 373.2383.

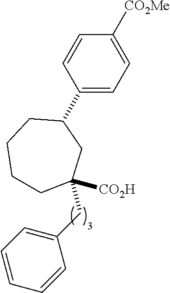

### cis-3-(4-(methoxycarbonyl)phenyl)-1-(3-phenylpropyl)cycloheptane-1-carboxylic acid (6h)

Following General Procedure 3 on 0.1 mmol scale. The product was obtained as colorless oil (18.5 mg, 47% yield). ¹H NMR (600 MHz, CDCl₃) δ 7.97-7.93 (m, 2H), 7.28-7.24 (m, 2H), 7.18 (dd, J=13.1, 7.7 Hz, 3H), 7.15-7.10 (m, 2H), 3.91 (s, 3H), 2.69-2.52 (m, 3H), 2.32 (ddd, J=14.7, 9.6, 7.7 Hz, 2H), 1.89 (t, J=15.5 Hz, 3H), 1.73 (td, J=12.6, 12.1, 4.7 Hz, 2H), 1.65 (ddd, J=16.3, 10.2, 3.9 Hz, 1H), 1.61-1.32 (m, 6H); ¹³C NMR (151 MHz, CDCl₃) δ 183.6, 167.2, 155.0, 142.1, 130.1, 128.5, 128.5, 127.9, 126.6, 126.0, 52.1, 49.1, 42.6, 41.0, 39.1, 39.1 36.8, 36.3, 30.7, 26.6, 23.9; HRMS (ESI-TOF) m/z Calcd for C₂₅H₃₁₀₄⁺[M+H]⁺ 395.2222, found 395.2220.

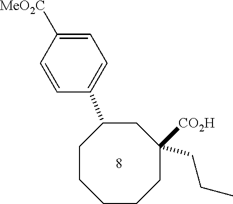 [SMILES: CCC[C@]1(C(=O)O)CCCCC[C@H](c2ccc(C(=O)OC)cc2)C1]

### cis-3-(4-(methoxycarbonyl)phenyl)-1-propylcyclooctane-1-carboxylic acid (6i)

Following General Procedure 4 on 0.1 mmol scale. The product was obtained as a colorless oil (13.3 mg, 40% yield). ¹H NMR (600 MHz, CDCl₃) δ 7.97-7.93 (m, 2H), 7.27-7.24 (m, 2H), 3.90 (s, 3H), 2.71 (tt, J=7.9, 3.4 Hz, 1H), 2.46-2.39 (m, 1H), 2.03-1.96 (m, 1H), 1.94 (dt, J=15.2, 1.7 Hz, 1H), 1.83-1.54 (m, 9H), 1.53-1.45 (m, 2H), 1.09-0.94 (m, 2H), 0.75 (t, J=7.3 Hz, 3H); ¹³C NMR (151 MHz, CDCl₃) δ 183.0, 167.3, 156.0, 130.1, 127.8, 127.1, 52.1, 50.0, 40.3, 39.9, 38.6, 35.3, 33.6, 27.3, 25.2, 24.3, 17.9, 14.5; HRMS (ESI-TOF) m/z Calcd for C₂₀H₂₉O₄⁺[M+H]⁺ 333.2066, found 333.2076.

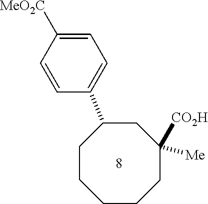 [SMILES: COC(=O)c1ccc([C@H]2CCCCC[C@](C)(C(=O)O)C2)cc1]

### cis-3-(4-(methoxycarbonyl)phenyl)-1-methylcyclooctane-1-carboxylic acid (6j)

Following General Procedure 4 on 0.1 mmol scale. The product was obtained as a colorless oil (13.7 mg, 45% yield). ¹H NMR (600 MHz, CDCl₃) δ 7.98-7.92 (m, 2H), 7.25 (d, J=8.3 Hz, 2H), 3.89 (s, 3H), 2.79-2.73 (m, 1H), 2.58 (dd, J=14.8, 7.5 Hz, 1H), 2.00-1.92 (m, 1H), 1.84-1.52 (m, 10H), 1.20 (s, 3H); ¹³C NMR (151 MHz, CDCl₃) δ 183.8, 167.3, 156.1, 130.1, 127.8, 127.1, 52.1, 45.8, 40.3, 39.6, 38.9, 33.4, 26.7, 26.0, 25.0, 24.6; HRMS (ESI-TOF) m/z Calcd for C₁₈H₂₅O₄⁺[M+H]305.1753, found 305.1767.

### REFERENCES

1. Xiao, K.-J., Lin, D. W., Miura, M., Zhu, R.-Y., Gong, W., Wasa, M., Yu, J.-Q. Palladium(II)-Catalyzed Enantioselective C(sp³)-H Activation Using a Chiral Hydroxamic Acid Ligand. J. Am. Chem. Soc. 136, 8138-8142 (2014).
2. Buchmann, B., Faulds, D., Guilford, W., Langer, G., Li, J., Lindenthal, B., Skuballa, W., Toschi, L. 9-Chloro-15-deoxyprostaglandin derivatives, process for their preparation and their use as medicaments. US 2007/203096 A1, Aug. 30, 2007.
3. Lehmann, J., Feichtinger, K., Ren, A., Semple, G. 5-HT2C Receptor Agonists and Compositions and Methods of Use. WO 2017/23679 A1, Feb. 9, 2017.
4. Lin, L. S., Liu, P. Acyclic hydrazides as cannabinoid receptor modulators. WO 2006/41797 A2, Mar. 20, 2006.
5. Volchkov, N. V., Zabolotskikh, A. V., Ignatenko, A. V., Nefedov, 0. M. Reaction of Fluorochlorocarbene with cyclopentadiene, indene, and their alkyl derivatives by phase transfer catalysis. Bulletin of the Academy of Sciences of the USSR Division of Chemical Science 39, 1458-1461 (1990).
6. Wei, Y., Yoshikai, N. Oxidative Cyclization of 2-Arylphenols to Dibenzofurans under Pd(II)/Peroxybenzoate Catalysis. Org. Lett. 13, 5504-5507 (2011).
7. Wang, C., Piel, I., Glorius, F. Palladium-Catalyzed Intramolecular Direct Arylation of Benzoic Acids by Tandem Decarboxylation/C—H Activation. J. Am. Chem. Soc. 131, 4194-4195 (2009).
8. Escale, R., Girard, J. P., Vidal, J. P., Teulade, J. C., Rossi, J. C., Chapat, J. P. Composes Cyclobutanes-1,3 Disubstitutes-V: Conformation en Solution des Acides Phenyl-3 et Cyclohexyl-3 Cyclobutanecarboxyliques. Tetrahedron 36, 1037-1041 (1980).

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

1. A method of transannular γ-methylene C(sp³)-H arylation of cycloalkane carboxylic acids, comprising treating a cycloalkyl carboxylic acid with an aryl or heteroaryl iodide in the presence of a Pd source and a ligand (L), which ligand is a quinuclidine-pyridone.

2. The method of claim 1, which method occurs in the presence of an oxidant and a base to provide a product according to the following reaction scheme: 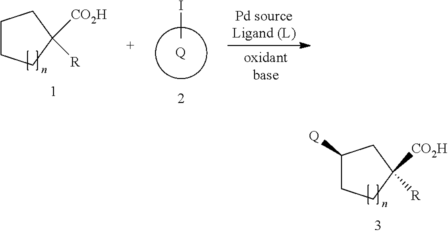
wherein:
Q is Ar or Het;
Ar is (C₆-C₁₀)aryl, optionally substituted with one or more R;
Het is unsaturated or partially unsaturated (C₅-C₈)heteroaryl, optionally substituted with one or more R^{a};
R is H or selected from the group consisting of (C₁-C₆)alkyl, (C₂₋₆)alkenyl, (C₂₋₆)alkynyl, (C₁-C₆)heteroalkyl, hetero (C₂₋₆)alkenyl, hetero (C₂₋₆)alkynyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃—C,)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, each independently and optionally substituted with one or more R^{a};
each R^{a} is —C(═O)O(C₁-C₆)alkyl, CN, NO₂, halo, OH, —C(═O)H, NH₂, (C₁-C₆)alkyl, halo (C₁-C₆)alkyl, (C₁-C₆)heteroalkyl, halo (C₁-C₆)heteroalkyl, (C₃—C,)cycloalkyl, (C₃—C,)heterocycloalkyl, (C₁-C₆)alkyl (C₃—C,)cycloalkyl, (C₁-C₆)alkyl (C₃—C,)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, —C(═O)(C₁-C₆)alkyl, oxo, Ts, —S(═O)₂F, —OS(═O)₂F, —S(═O)₂(C₁-C₆)alkyl, or_-C(═O)NH(C₁-C₆)alkyl; and
n is 1, 2, 3, or 4.

3. The method of claim 2, wherein n is 1 and the ligand is selected from the group consisting of:

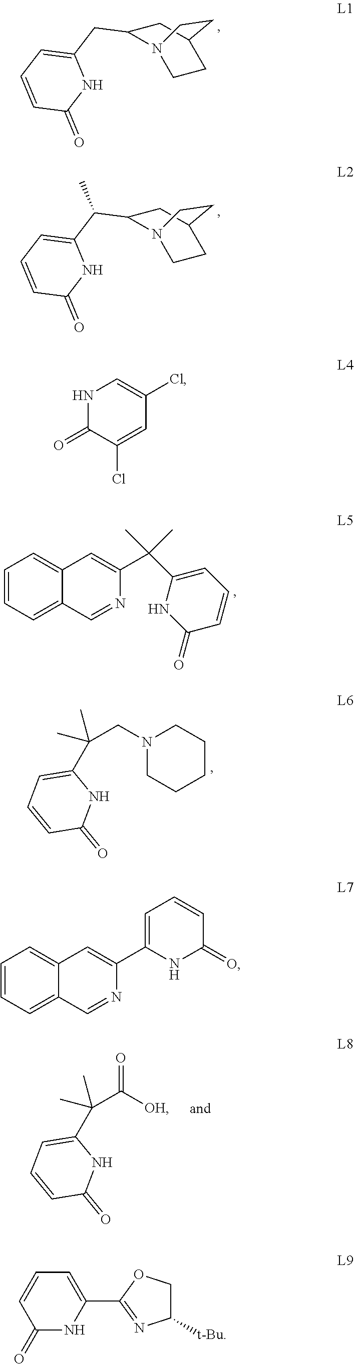

4. The method of claim 2, wherein n is 2 and the ligand is selected from the group consisting of:

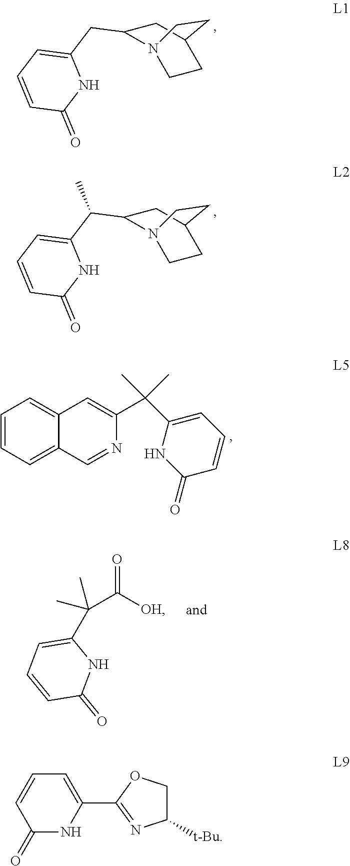

5-10. (canceled)

11. The method of claim 2, wherein Q is Ar optionally substituted with one or more R^{a}.

12. The method of claim 11, wherein Ar is Ph optionally substituted with one or more R^{a}.

13-27. (canceled)

28. The method of claim 2, wherein Q is Het optionally substituted with one or more R^{a}.

29. The method of claim 28, wherein Het is pyridinyl, thiophenyl, pyrimidinyl, 1H-indolyl, quinolinyl, benzo[d]thiazolyl, indazolyl, or pyrazolyl.

30-52. (canceled)

53. The method of claim 2, wherein R is (C₁-C₆)alkyl.

54-64. (canceled)

65. The method of claim 1, wherein the Pd source is Pd(OAc)₂, PdCl₂allyl₂, Pd(TFA)₂, PdCl₂(PhCN)₂, PdCl₂(ethylenediamine), PdCl₂(PPh₃)₂, Pd(dba)₃, or PdCl₂(dppf).

66-72. (canceled)

73. The method of claim 2, wherein the oxidant is AgOAc, Ag₂CO₃, Ag₂O, AgNO₃, AgPO₄, AgTFA, or Na₂CO₃·1.5H₂O₂.

74-79. (canceled)

80. The method of claim 2, wherein the base is Na₂CO₃, Li₂CO₃, K₂CO₃, Cs₂CO₃, NaHCO₃, K₂HPO₄, KH₂PO₄, or Na₃PO₄.

81-87. (canceled)

88. The method of claim 2, wherein the base is Na₂HPO₄·7H₂O, t-BuOK, CH₃CO₂Na, Na₂SO₃, Li₃PO₄, KF, t-AmONa, HCO₂Na, HCO₂K, CH₃CO₂Na, or CH₃CO₂K.

89-122. (canceled)

123. The method of claim 2, wherein the product is cis-1-(3-fluoro-2-methylphenyl)-3-(1-methyl-1H-indazol-5-yl)cyclopentane-1-carboxylic acid, cis-1-(3-fluoro-2-methylphenyl)-3-(1-methyl-1H-pyrazol-5-yl)cyclopentane-1-carboxylic acid, cis-1-(3-fluoro-2-methylphenyl)-3-(quinolin-6-yl)cyclopentane-1-carboxylic acid, cis-3-(1-(difluoromethyl)-1H-pyrazol-4-yl)-1-(3-fluoro-2-methylphenyl)cyclopentane-1-carboxylic acid, cis-1-(3-fluoro-2-methylphenyl)-3-(1-tosyl-1H-indol-5-yl)cyclopentane-1-carboxylic acid, cis-3-(5-acetylthiophen-2-yl)-1-(3-fluoro-2-methylphenyl)cyclopentane-1-carboxylic acid, cis-3-phenylcyclopentane-1-carboxylic acid, cis-3-(4-chlorophenyl)cyclopentane-1-carboxylic acid, cis-3-(4-methoxyphenyl)cyclopentane-1-carboxylic acid, cis-3-phenylcyclopentane-1-carboxylic acid, cis-3-(4-(trifluoromethyl)phenyl)cyclopentane-1-carboxylic acid, cis-3-(3-(trifluoromethyl)phenyl)cyclopentane-1-carboxylic acid, cis-3-(2-(trifluoromethyl)phenyl)cyclopentane-1-carboxylic acid, cis-3-(2-chloropyridin-4-yl)cyclopentane-1-carboxylic acid, cis-1-(3-fluoro-2-methylphenyl)-3-(1-tosyl-1H-indol-5-yl)cyclopentane-1-carboxylic acid, or cis-3-(5-acetylthiophen-2-yl)-1-(3-fluoro-2-methylphenyl)cyclopentane-1-carboxylic acid.

124-134. (canceled)

135. The method of claim 2, wherein the product is cis-3-(4-cyanophenyl)-1-propylcyclopentane-1-carboxylic acid, cis-3-(4-nitrophenyl)-1-propylcyclopentane-1-carboxylic acid, cis-3-(4-chlorophenyl)-1-propylcyclopentane-1-carboxylic acid, cis-3-(4-bromophenyl)-1-propylcyclopentane-1-carboxylic acid, cis-1-propyl-3-(4-(trifluoromethyl)phenyl)cyclopentane-1-carboxylic acid, cis-3-(4-fluorophenyl)-1-propylcyclopentane-1-carboxylic acid, cis-3-(4-methoxyphenyl)-1-propylcyclopentane-1-carboxylic acid, cis-3-(4-formylphenyl)-1-propylcyclopentane-1-carboxylic acid, cis-3-(4-acetylphenyl)-1-propylcyclopentane-1-carboxylic acid, cis-3-(3-(methoxycarbonyl)phenyl)-1-propylcyclopentane-1-carboxylic acid, cis-3-(3-nitrophenyl)-1-propylcyclopentane-1-carboxylic acid, cis-1-propyl-3-(3-(trifluoromethyl)phenyl)cyclopentane-1-carboxylic acid, cis-3-(3-fluorophenyl)-1-propylcyclopentane-1-carboxylic acid, cis-3-(2-fluorophenyl)-1-propylcyclopentane-1-carboxylic acid, cis-1-propyl-3-(2-(trifluoromethyl)phenyl)cyclopentane-1-carboxylic acid, cis-3-(2-cyanophenyl)-1-propylcyclopentane-1-carboxylic acid, cis-3-(3,5-bis(trifluoromethyl)phenyl)-1-propylcyclopentane-1-carboxylic acid, cis-3-(naphthalen-1-yl)-1-propylcyclopentane-1-carboxylic acid, cis-1-propyl-3-(2-(trifluoromethyl)pyridin-4-yl)cyclopentane-1-carboxylic acid, cis-3-(2-chloropyridin-4-yl)-1-propylcyclopentane-1-carboxylic acid, cis-3-(2-bromopyridin-4-yl)-1-propylcyclopentane-1-carboxylic acid, cis-3-(2-methylpyridin-4-yl)-1-propylcyclopentane-1-carboxylic acid, cis-3-(2,6-dichloropyridin-4-yl)-1-propylcyclopentane-1-carboxylic acid, cis-3-(2-chloro-6-(trifluoromethyl)pyridin-4-yl)-1-propylcyclopentane-1-carboxylic acid, cis-3-(6-chloro-5-(trifluoromethyl)pyridin-3-yl)-1-propylcyclopentane-1-carboxylic acid, cis-3-(6-chloropyridin-3-yl)-1-propylcyclopentane-1-carboxylic acid, cis-3-(6-fluoropyridin-3-yl)-1-propylcyclopentane-1-carboxylic acid, cis-3-(4-(1H-pyrrol-1-yl)phenyl)-1-propylcyclopentane-1-carboxylic acid, cis-3-(4-(1,3-dioxo-1H-benzo[de]isoquinolin-2(3H)-yl)phenyl)-1-propylcyclopentane-1-carboxylic acid, cis-1-propyl-3-(thiophen-2-yl)cyclopentane-1-carboxylic acid, cis-3-(5-acetylthiophen-2-yl)-1-propylcyclopentane-1-carboxylic acid, cis-3-(2-methylbenzo[d]thiazol-6-yl)-1-propylcyclopentane-1-carboxylic acid, cis-1-propyl-3-(quinazolin-6-yl)cyclopentane-1-carboxylic acid, cis-1-propyl-3-(quinolin-6-yl)cyclopentane-1-carboxylic acid, cis-3-(2,4-dimethoxypyrimidin-5-yl)-1-propylcyclopentane-1-carboxylic acid, or cis-1-propyl-3-(1-tosyl-1H-indol-5-yl)cyclopentane-1-carboxylic acid.

136. (canceled)

137. The method of claim 2, wherein the product is cis-3-(4-(methoxycarbonyl)phenyl)-1-propylcyclopentane-1-carboxylic acid, cis-3-(4-(methoxycarbonyl)phenyl)-1-phenylcyclopentane-1-carboxylic acid, cis-1-(4-chlorophenyl)-3-(4-(methoxycarbonyl)phenyl)cyclopentane-1-carboxylic acid, cis-3-(4-(methoxycarbonyl)phenyl)-1-(p-tolyl)cyclopentane-1-carboxylic acid, cis-3-(4-(methoxycarbonyl)phenyl)-1-(4-methoxyphenyl)cyclopentane-1-carboxylic acid, cis-1-(2-fluorophenyl)-3-(4-(methoxycarbonyl)phenyl)cyclopentane-1-carboxylic acid, cis-1-(4-fluorophenyl)-3-(4-(methoxycarbonyl)phenyl)cyclopentane-1-carboxylic acid, cis-1-(3-fluoro-2-methylphenyl)-3-(4-(methoxycarbonyl)phenyl)cyclopentane-1-carboxylic acid, cis-3-(4-(methoxycarbonyl)phenyl)-1-methylcyclopentane-1-carboxylic acid, cis-1-ethyl-3-(4-(methoxycarbonyl)phenyl)cyclopentane-1-carboxylic acid, cis-1-benzyl-3-(4-(methoxycarbonyl)phenyl)cyclopentane-1-carboxylic acid, cis-1-(3-(tert-butoxy)-3-oxopropyl)-3-(4-(methoxycarbonyl)phenyl)cyclopentane-1-carboxylic acid, cis-3-(4-(methoxycarbonyl)phenyl)-1-(3-phenylpropyl)cyclopentane-1-carboxylic acid, cis-1-(cyclohexylmethyl)-3-(4-(methoxycarbonyl)phenyl)cyclopentane-1-carboxylic acid, cis-1-(4-chlorobutyl)-3-(4-(methoxycarbonyl)phenyl)cyclopentane-1-carboxylic acid, cis-3-(4-(methoxycarbonyl)phenyl)-1-(3-methoxypropyl)cyclopentane-1-carboxylic acid, or cis-3-(4-(methoxycarbonyl)phenyl)cyclopentane-1-carboxylic acid.

138. The method of claim 2, which method occurs according to the following reaction scheme: 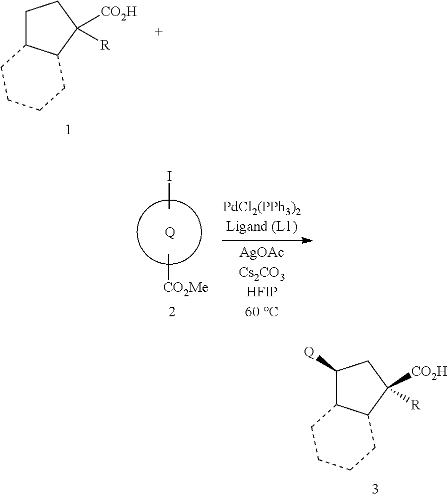
wherein [symbol] indicates single or double bonds.

139. The method of claim 138, wherein compound 3 is rel-(1S,3R,3aR,7aR)-3-(4-(methoxycarbonyl)phenyl)-1-methyloctahydro-1H-indene-1-carboxylic acid or cis-3-(4-(methoxycarbonyl)phenyl)-1-propyl-2,3-dihydro-1H-indene-1-carboxylic acid.

140. (canceled)

141. The method of claim 2, wherein the product is cis-3-(4-(methoxycarbonyl)phenyl)-1-propylcyclohexane-1-carboxylic acid, cis-3-(4-(methoxycarbonyl)phenyl)-1-methylcyclohexane-1-carboxylic acid, cis-1-ethyl-3-(4-(methoxycarbonyl)phenyl)cyclohexane-1-carboxylic acid, cis-1-isobutyl-3-(4-(methoxycarbonyl)phenyl)cyclohexane-1-carboxylic acid, cis-1-(3-((tert-butyldimethylsilyl)oxy)propyl)-3-(4-(methoxycarbonyl)phenyl)cyclohexane-1-carboxylic acid, cis-1-(cyclohexylmethyl)-3-(4-(methoxycarbonyl)phenyl)cyclohexane-1-carboxylic acid, cis-3-(4-(methoxycarbonyl)phenyl)-1-(3-phenoxypropyl)cyclohexane-1-carboxylic acid, cis-1-benzyl-3-(4-(methoxycarbonyl)phenyl)cyclohexane-1-carboxylic acid, cis-3-(4-(methoxycarbonyl)phenyl)-1-phenylcyclohexane-1-carboxylic acid, cis-3-(4-(methoxycarbonyl)phenyl)-1-(p-tolyl)cyclohexane-1-carboxylic acid, cis-3-(4-(methoxycarbonyl)phenyl)-1-(4-methoxyphenyl)cyclohexane-1-carboxylic acid, cis-5-(4-(methoxycarbonyl)phenyl)-1,3,3-trimethylcyclohexane-1-carboxylic acid, cis-2-(4-(methoxycarbonyl)phenyl)-4-propyltetrahydro-2H-pyran-4-carboxylic acid, (2R,4R,4aS,6aR,9S,11aR,11bS)-2-(4-(methoxycarbonyl)phenyl)-4,9,11b-trimethyl-8-oxotetradecahydro-6a,9-methanocyclohepta[a]naphthalene-4-carboxylic acid, cis-3-(4-(methoxycarbonyl)phenyl)-1-propylcycloheptane-1-carboxylic acid, cis-3-(4-(methoxycarbonyl)phenyl)-1-methylcycloheptane-1-carboxylic acid, cis-1-ethyl-3-(4-(methoxycarbonyl)phenyl)cycloheptane-1-carboxylic acid, cis-3-(4-(methoxycarbonyl)phenyl)-1-(3-methoxypropyl)cycloheptane-1-carboxylic acid, cis-1-(3-((tert-butyldimethylsilyl)oxy)propyl)-3-(4-(methoxycarbonyl)phenyl)cycloheptane-1-carboxylic acid, cis-1-(5-chloropentyl)-3-(4-(methoxycarbonyl)phenyl)cycloheptane-1-carboxylic acid, cis-1-(cyclohexylmethyl)-3-(4-(methoxycarbonyl)phenyl)cycloheptane-1-carboxylic acid, or cis-3-(4-(methoxycarbonyl)phenyl)-1-(3-phenylpropyl)cycloheptane-1-carboxylic acid, or cis-3-(4-(methoxycarbonyl)phenyl)-1-propylcyclooctane-1-carboxylic acid, or cis-3-(4-(methoxycarbonyl)phenyl)-1-methylcyclooctane-1-carboxylic acid.

142. (canceled)

143. (canceled)

144. (canceled)

145. (canceled)

146. A compound, which compound is: 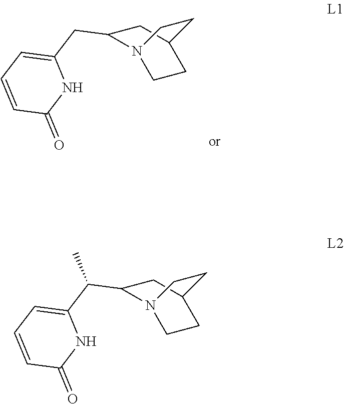 [SMILES: O=c1cccc(CC2CC3CCN2CC3)[nH]1 ; C[C@@H](c1cccc(=O)[nH]1)C1CC2CCN1CC2]
or a pharmaceutically acceptable salt thereof.

147-150. (canceled)
